(19) United States
(12) Patent Application Publication
(10) Pub. No.: US 2025/0387195 A1
(43) Pub. Date: Dec. 25, 2025

(54) MULTI-INSTRUMENT IDENTIFICATION DEVICES, SYSTEMS, AND METHODS

(71) Applicant: Stratus Medical, LLC, Magnolia, TX (US)

(72) Inventors: Bret Boudousquie, The Woodlands, TX (US); Eric Hyman, Wakefield, MA (US); Steve Lepke, Wakefield, MA (US); Steve Barry Woolfson, Boston, MA (US)

(73) Assignee: Stratus Medical, LLC, Magnolia, TX (US)

(21) Appl. No.: 19/312,295

(22) Filed: Aug. 27, 2025

Related U.S. Application Data
(63) Continuation of application No. PCT/US24/17775, filed on Feb. 28, 2024.
(60) Provisional application No. 63/449,045, filed on Feb. 28, 2023.

(51) Int. Cl. A61B 90/98; A61B 17/00; A61B 18/00; A61B 18/12; A61B 34/00

(52) U.S. Cl. CPC A61B 90/98; A61B 18/1206; A61B 34/25; A61B 2017/00115; A61B 2018/00178

(57) ABSTRACT

A system can include an assembly that includes a connector, a medical instrument, an instrument indicator, and a memory device. The system can further include a control machine that includes a first port, a second port, and a controller. The controller can, when the connector of the assembly is attached to the first port, obtain the stored information from the memory device; make a determination based on the stored information that the assembly comprises the instrument indicator; in response to said determination, control a first port indicator to display light of a first color and control a second port indicator to display light of a second color that is different from the first color; control the instrument indicator to display light of the first color; and control the medical instrument to perform a function for treatment of a patient.

### RELATED APPLICATIONS

This application is a continuation of International Application No. PCT/US2024/017775, filed on Feb. 28, 2024, titled MULTI-INSTRUMENT IDENTIFICATION DEVICES, SYSTEMS, AND METHODS, which claims priority to U.S. Provisional Patent Application No. 63/449,045, filed on Feb. 28, 2023, titled MULTI-INSTRUMENT IDENTIFICATION DEVICES, SYSTEMS, AND METHODS, the entire contents of each of which are hereby incorporated by reference herein.

### TECHNICAL FIELD

Certain embodiments described herein relate generally to identifying medical instruments and related systems and methods, and relate more particularly to devices, systems, and methods for facilitating identification of multiple medical instruments coupled with a control machine, such as, for example, multiple radiofrequency probes coupled with a radiofrequency generator.

### BACKGROUND

In various contexts, multiple instruments can be coupled with a control machine, such as via a plurality of cables that are uniquely associated with each instrument. For example, numerous medical procedures (surgical, monitoring, and/or diagnostic procedures, for example) involve the use of multiple instruments that are each coupled with the control machine via a separate cable. In many instances, determining which instrument is coupled with which port, connector, channel, etc. of the control machine can be a frustrating and/or time-consuming endeavor. To date, devices, systems, and methods for addressing these and related issues suffer from a number of drawbacks. Embodiments disclosed herein remedy, ameliorate, or avoid one or more of these drawbacks and/or can be advantageously time saving and/or can be readily implemented and/or understood by a user.

### BRIEF DESCRIPTION OF THE DRAWINGS

The written disclosure herein describes illustrative embodiments that are non-limiting and non-exhaustive. Reference is made to certain of such illustrative embodiments that are depicted in the figures, in which:

FIG. 1 is an elevation view, with certain components depicted schematically, of an embodiment of a system that includes a control machine that includes multiple ports and multiple instrument assemblies that can be coupled interchangeably with any of the ports of the control machine;

FIG. 2A is an enlarged cross-sectional schematic view of an embodiment of a port of the control machine of FIG. 1;

FIG. 2B is an elevation view of an embodiment of an instrument assembly that includes an embodiment of a connector that is couplable with any of the ports of the control machine of FIG. 1, and further includes a medical instrument at a distal end thereof;

FIG. 3A is an enlarged cross-sectional schematic view of another embodiment of a port another embodiment of a control machine, such as the control machine depicted in FIG. 1;

FIG. 3B is an elevation view of an embodiment of an instrument assembly that includes an embodiment of a connector couplable with ports such as that depicted in FIG. 3A;

FIG. 3C is an elevation view of another embodiment of an instrument assembly that includes another embodiment of a connector couplable with ports such as that depicted in FIG. 3A;

FIG. 3D is an elevation view of yet another embodiment of an instrument assembly that includes yet another embodiment of a connector couplable with ports such as that depicted in FIG. 3A;

FIG. 4 is an enlarged elevation view of a portion of the system of FIG. 1 in which a first instrument assembly having a medical instrument at a distal end thereof is coupled with a first port of the control machine, and in which an indicator associated with the first port and a further indicator associated with the instrument convey information indicating correspondence of the instrument to the first port;

FIG. 5A is another enlarged elevation view of a portion of the system of FIG. 1 in which four separate instrument assemblies are coupled with four separate ports of the control machine, and in which individual indicators associated with each of the four ports of the control machine, respectively, and individual indicators associated with each of the instrument assemblies, respectively, convey information indicating correspondence of the instrument at a distal end of each instrument assembly to the respective port with which that instrument is coupled;

FIG. 5B is a further enlarged elevation view of a portion of FIG. 5A that provides greater detail regarding two of the ports and two of the instrument assemblies that are individually coupled with these ports;

FIG. 6 is an elevation view of another embodiment of a system that includes a control machine having four instrument ports and four instrument assemblies that can be coupled interchangeably with any of the ports, in which two separate indicators associated with each of the four ports of the control machine, respectively, and individual indicators associated with each of the instrument assemblies, respectively, convey information indicating correspondence of the instrument at a distal end of each instrument assembly to the respective port with which that instrument is coupled;

FIG. 7 is an elevation view of another embodiment of a system that includes a control machine having four instrument ports and four instrument assemblies that can be coupled interchangeably with any of the ports, in which a separate indicator associated with each of the four ports of the control machine, respectively, and individual indicators associated with each of the instrument assemblies, respectively, convey information indicating correspondence of the instrument at a distal end of each assembly to the respective port with which that instrument is coupled;

FIG. 8 is an elevation view of another embodiment of a system that includes a control machine having four instrument ports and four instrument assemblies that can be coupled interchangeably with any of the ports, in which a separate indicator associated with each of the four ports of the control machine, respectively, and individual indicators associated with each of the instrument assemblies, respectively, convey information indicating correspondence of the instrument at a distal end of each assembly to the respective port with which that instrument is coupled;

FIG. 9 is an elevation view, with certain components depicted schematically, of another embodiment of a system that includes a control machine that includes multiple ports and adapters that can be coupled interchangeably with any of the ports of the control machine, the system further including a plurality of instrument assemblies that can be coupled interchangeably with any of the adapters, each adapter including an indicator configured to correspond with an indicator of the instrument assembly coupled thereto;

FIG. 10A is a schematic front view of an embodiment of a port that is compatible with embodiments of the control machine of FIG. 9;

FIG. 10B is a schematic front view of another embodiment of a port that is compatible with embodiments of the control machine of FIG. 9;

FIG. 10C is a schematic front view of yet another embodiment of a port that is compatible with embodiments of the control machine of FIG. 9;

FIG. 11A is a schematic end-on view of an embodiment of a proximal connector of an embodiment of an adapter that is compatible with certain embodiments of the control machine of FIG. 9;

FIG. 11B is a schematic end-on view of another embodiment of a proximal connector of another embodiment of an adapter that is compatible with certain embodiments of the control machine of FIG. 9;

FIG. 11C is a schematic end-on view of yet another embodiment of a proximal connector of yet another embodiment of an adapter that is compatible with certain embodiments of the control machine of FIG. 9;

FIG. 12A is a schematic end-on view of an embodiment of a distal connector of an embodiment of an adapter that is compatible with certain embodiments of the control machine of FIG. 9;

FIG. 12B is a schematic end-on view of another embodiment of a distal connector of another embodiment of an adapter that is compatible with certain embodiments of the control machine of FIG. 9;

FIG. 12C is a schematic end-on view of yet another embodiment of a distal connector of yet another embodiment of an adapter that is compatible with certain embodiments of the control machine of FIG. 9;

FIG. 13A is an elevation view of an embodiment of an adapter that is compatible with, e.g., embodiments of the system of FIG. 9;

FIG. 13B is a schematic diagram depicting various components of the adapter of FIG. 13A;

FIG. 14A is an elevation view of another embodiment of an adapter that is compatible with, e.g., embodiments of the system of FIG. 9;

FIG. 14B is a schematic diagram depicting various components of the adapter of FIG. 14A;

FIG. 15A is an elevation view of another embodiment of an adapter that is compatible with, e.g., embodiments of the system of FIG. 9;

FIG. 15B is a schematic diagram depicting various components of the adapter of FIG. 15A;

FIG. 16A is an elevation view of another embodiment of an adapter that is compatible with, e.g., embodiments of the system of FIG. 9;

FIG. 16B is a schematic diagram depicting various components of the adapter of FIG. 16A;

FIG. 17A is an elevation view of another embodiment of an adapter that is compatible with, e.g., embodiments of the system of FIG. 9;

FIG. 17B is a schematic diagram depicting various components of the adapter of FIG. 17A;

FIG. 18A is an elevation view of another embodiment of an adapter that is compatible with, e.g., embodiments of the system of FIG. 9;

FIG. 18B is a schematic diagram depicting various components of the adapter of FIG. 18A;

FIG. 19 is an elevation view of another embodiment of an adapter that is compatible with, e.g., embodiments of the system of FIG. 9;

FIG. 20 is an elevation view of another embodiment of an adapter that is compatible with, e.g., embodiments of the system of FIG. 9;

FIG. 21A is an elevation view of another embodiment of an adapter that is compatible with, e.g., embodiments of the system of FIG. 9;

FIG. 21B is a schematic diagram depicting various components of the adapter of FIG. 21A;

FIG. 22 is an elevation view of another embodiment of an adapter that is compatible with, e.g., embodiments of the system of FIG. 9;

FIG. 23 is a schematic diagram depicting various components of the adapter of FIG. 22;

FIG. 24 is an elevation view of another embodiment of an adapter that is compatible with, e.g., embodiments of the system of FIG. 9;

FIG. 25 is an elevation view of another embodiment of an adapter that is compatible with, e.g., embodiments of the system of FIG. 9;

FIG. 26 is an elevation view of another embodiment of an adapter that is compatible with, e.g., embodiments of the system of FIG. 9;

FIG. 27 is an elevation view of another embodiment of an adapter that is compatible with, e.g., embodiments of the system of FIG. 9;

FIG. 28 is an elevation view of another embodiment of an adapter that is compatible with, e.g., embodiments of the system of FIG. 10;

FIG. 29 is an embodiment of a RF probe assembly that is compatible with embodiments of the system of FIG. 10 and/or embodiments of one or more of the adapters depicted in FIGS. 9 and 11A-28;

FIG. 30 is an elevation view, with certain components depicted schematically, of another embodiment of a system that includes a control machine that includes multiple ports, the system further including an adapter that includes multiple connectors that are each configured to individually couple with one of the ports of the control machine and further includes multiple ports, the ports of the adapter being configured to interchangeably couple with any of a respective one of a plurality of instrument assemblies, with each adapter including an indicator configured to correspond with an indicator of the instrument assembly coupled thereto;

FIG. 31 is a front view of another embodiment of an adapter that is compatible with embodiments of the system of FIG. 30;

FIG. 32 is an elevation view of another embodiment of an adapter;

FIG. 33 is an elevation view of yet another embodiment of an adapter;

FIG. 34 is a an elevation view, with certain components depicted schematically, of another embodiment of a system that includes a control machine that includes multiple ports and multiple instrument assemblies that can be coupled interchangeably with any of the ports of the control machine;

FIG. 35 is an enlarged elevation view of a portion of the system of FIG. 34 in which four separate instrument assemblies are coupled with four separate ports of the control machine, and in which individual indicators associated with each of the four ports of the control machine, respectively, and individual indicators associated with each of the instrument assemblies, respectively, convey information indicating correspondence of the instrument at a distal end of each instrument assembly to the respective port with which that instrument is coupled;

FIG. 36 is a schematic diagram depicting various communicative and power couplings among certain components of the system of FIG. 35;

FIG. 37 is an enlarged cross-sectional schematic view of an embodiment of a port of the control machine of FIG. 35;

FIG. 38 is a front view of an embodiment of a port that is compatible with embodiments of the control machine of FIG. 9;

FIG. 39A is an elevation view of an embodiment of an instrument assembly that includes an embodiment of a connector that is couplable with any of the ports of the control machine of FIG. 35, and further includes a medical instrument at a distal end thereof;

FIG. 39B is a schematic end-on view of an embodiment of a proximal connector that is compatible with certain embodiments of the instrument assembly of FIG. 39A;

FIG. 40 is a schematic diagram of an embodiment of the instrument assembly of FIG. 39A that depicts various communicative and power couplings among components of the instrument assembly;

FIG. 41A is an elevation view of another embodiment of an instrument assembly that is couplable with any of the ports of certain embodiments of the control machine of FIG. 35;

FIG. 41B is an elevation view of yet another embodiment of an instrument assembly that is couplable with any of the ports of certain embodiments of the control machine of FIG. 35;

FIG. 42 depicts an illustrative control screen via which a monopolar sensory stimulation procedure is controllable, the control screen being displayable on a touchscreen of certain embodiments of the control machine of FIG. 35;

FIG. 43 depicts an illustrative control screen via which a monopolar motor stimulation procedure is controllable, the control screen being displayable on a touchscreen of certain embodiments of the control machine of FIG. 35;

FIG. 44 depicts an illustrative control screen via which settings for a monopolar procedure are selectable, the control screen being displayable on a touchscreen of certain embodiments of the control machine of FIG. 35;

FIG. 45 depicts an illustrative control screen via which the monopolar lesion procedure associated with FIG. 44 is controllable, the control screen being displayable on a touchscreen of certain embodiments of the control machine of FIG. 35;

FIG. 46 depicts an illustrative control screen via which a bipolar lesion procedure is controllable, the control screen being displayable on a touchscreen of certain embodiments of the control machine of FIG. 35;

FIG. 47 depicts an illustrative control screen via which system settings can be selected and/or adjusted, including settings related to indicators associated with ports of the control machine and/or instrument assemblies coupled thereto;

FIG. 48 is a diagram depicting operational stages of an illustrative method that can be conducted via embodiments of a system such as that depicted in FIG. 35;

FIG. 49 is a diagram depicting operational stages of another illustrative method that can be conducted via embodiments of a system such as that depicted in FIG. 35;

FIG. 50 is a diagram depicting operational stages of yet another illustrative method that can be conducted via embodiments of a system such as that depicted in FIG. 35;

FIG. 51A is an elevation view of an embodiment of an adapter that is connectable at one end with a port of certain embodiments of control machines and is connectable at another end with embodiments of an instrument assembly;

FIG. 51B is a schematic diagram depicting various components of the adapter of FIG. 51A;

FIG. 52A is an elevation view of another embodiment of an adapter that is connectable at one end with a port of certain embodiments of control machines and is connectable at another end with embodiments of an instrument assembly; and

FIG. 52B s a schematic diagram depicting various components of the adapter of FIG. 52A.

### DETAILED DESCRIPTION

Embodiments of the present disclosure relate generally to systems in which a control machine is simultaneously couplable with multiple instruments. The instruments can be controlled by the control machine so as to be operative at a distance from the control machine. For example, in some instances, each of the instruments includes or is otherwise associated with a separate cable that can be coupled with the control machine, such as via a connector that attaches to an individual port of the control machine. Thus, for example, the control machine can include multiple ports, each port being couplable with an individual instrument assembly (which may include, e.g., a connector, a cable, and an instrument). In some instances, each of the ports may be operable with any of the various instruments. Stated otherwise, the instruments may be coupled interchangeably with any of the ports of the control machine. As one illustrative example, embodiments of a radiofrequency (RF) neurotomy system can include a control machine, such as an RF generator, and a plurality of RF probe assemblies that are simultaneously coupled with the control machine and used to create lesions within the patient, such as for purposes of ablating one or more nerves. In certain embodiments, each RF probe assembly includes a cable, a connector at a proximal end of the cable, and an RF probe at a distal end of the connector. The connector of each probe can be coupled with a separate port of the generator. In some embodiments, each RF probe assembly is substantially identical to the remaining RF probe assemblies, and each may be used interchangeably with any port of the generator.

With multiple interchangeable RF probes being coupled to the RF generator and spaced from the RF generator during use, it can become difficult to discern which RF probe corresponds with which port of the RF generator. In many instances, the generator may be a non-sterile device that should be maintained outside of a surgical field of a patient, whereas the RF probes themselves can be coupled with RF needles that are used within the surgical field, and thus the RF probe assembly cables may be relatively long to accommodate this desired distancing between the generator and the RF probes. In many instances, elongated cables that extend between the connector and the RF probe of each probe assembly can complicate determining which RF probe is associated with which port, as the cables can become intertwined, coiled, jumbled, overlap one another, and/or otherwise make the various port/probe associations difficult to discern. Embodiments disclosed herein can resolve this and/or other issues associated with systems that include a control machine and multiple instruments that are controllable by the control machine. For example, in some embodiments, a control machine includes one or more separate indicators associated with each port (e.g., at or near each port and/or on a display screen); each instrument assembly includes one or more separate indicators, which may be located substantially at a distal end thereof; and the one or more indicators corresponding to each port of the control machine correspond with, respectively, the one or more indicators of each instrument assembly to assist a user in determining which of the ports each of the instrument assemblies is coupled with. For example, each port may have a differently colored indicator (e.g., a differently colored light element), and the indicator of each instrument assembly can correspond with the color of the respective port to which the instrument assembly is coupled.

Some embodiments include control machines that include a plurality of indicators associated with a corresponding plurality of ports. The control machines can include a controller or control circuitry and/or other suitable componentry that is configured to communicate with an instrument assembly that is brought into proximity with and/or is connected to one of the ports. For example, the control machine can be configured to communicate wirelessly with the instrument assembly when the instrument assembly is brought near to a port, such as, for example, via a radiofrequency identification (RFID) reader that communicates with an RFID tag, which may be positioned within the instrument assembly. In other or further embodiments, the instrument assembly includes a data storage element from which the control machine can retrieve or otherwise obtain stored data when the instrument assembly is connected to a port. For example, in some embodiments, the instrument assembly can include an electrically erasable programmable read-only memory (EEPROM) device that stores information indicative of the instrument assembly having a controllable indicator. The control machine can read this stored information and carry out various activities as a result, such as controlling one or more of the port indicators and/or controlling the instrument assembly indicator, in manners such as described more fully herein.

In some embodiments, the control machine can permit a user to control one or more aspects of dynamic indicators, such as port indictors associated with ports of the control machine and/or instrument assembly indicators associated with the instrument assemblies. For example, in various embodiments, the control machine includes an actuator by which a user can control operation of one or more indicators. In some embodiments, the actuator is a physical dedicated actuator, such as a physical button. In other or further embodiments, the actuator comprises a virtual actuator, such as a virtual button, that is displayed via the touchscreen and is actuatable via interaction with the touchscreen.

In some embodiments, a system can include adapters that are coupled to the individual ports of a generator or other control machine. The adapters each can include one or more indicators that can provide unique identification for each of the ports of the control machine to which the adapters are coupled. Further, the adapters can be coupled with instrument assemblies that each have one or more indicators. The indicators of the adapters can correspond with the respective indicators of the instrument assemblies coupled thereto to provide a correspondence or association between each port of the control machine and the respective instrument assembly that is coupled thereto by way of one of the adapters. For example, each adapter that is attached to a separate port of a control machine via a proximal connector of the adapter may display a different color of light via the indicator of the adapter, and the adapter can further control an indicator of an instrument assembly that is attached to a distal connector of the adapter.

Stated otherwise, in some embodiments, each adapter includes one or more indicators capable of providing individualized identification of each port of a control machine. The adapters can be configured for use with control machines that do not or cannot control indicators of specialized instrument assemblies with port-identification or port-correspondence features (e.g., instrument assemblies that have color-controllable lights at a distal end thereof). The adapters thus, in some instances, my permit retrofitting of certain control machines for use with identification-equipped instrument assemblies, or stated otherwise, with instrument assemblies that include indicators, such as controllable, dynamic, or changeable indicators. In some embodiments, an adapter can include an indicator to uniquely identify a port of the control machine to which it is attached, and in further embodiments, the adapter can control an indicator of an instrument assembly that is attached thereto to create an association (e.g., a match) between the indicators of the adapter and the instrument assembly. This association can assist a user in determining which instrument assembly is coupled with which port of the control machine.

By way of example, in some embodiments, an adapter can include a colored light indicator (in further embodiments, the colored light may be dynamically changeable or selectable). In further embodiments, an instrument assembly that is couplable with the adapter can include a colored light indicator, e.g., at a distal end thereof. The adapter, in some instances, can control one or more of the color of its own light indicator and the color of the light indicator of the instrument assembly to achieve matching colors and thereby create a visually perceptible association between the instrument assembly and the port to which the instrument assembly is coupled.

In certain embodiments, advantages of devices, systems, and/or methods disclosed herein can include ease of use and/or reduction of procedure duration. One or more of these and/or other advantages of various embodiments will be understood from the present disclosure.

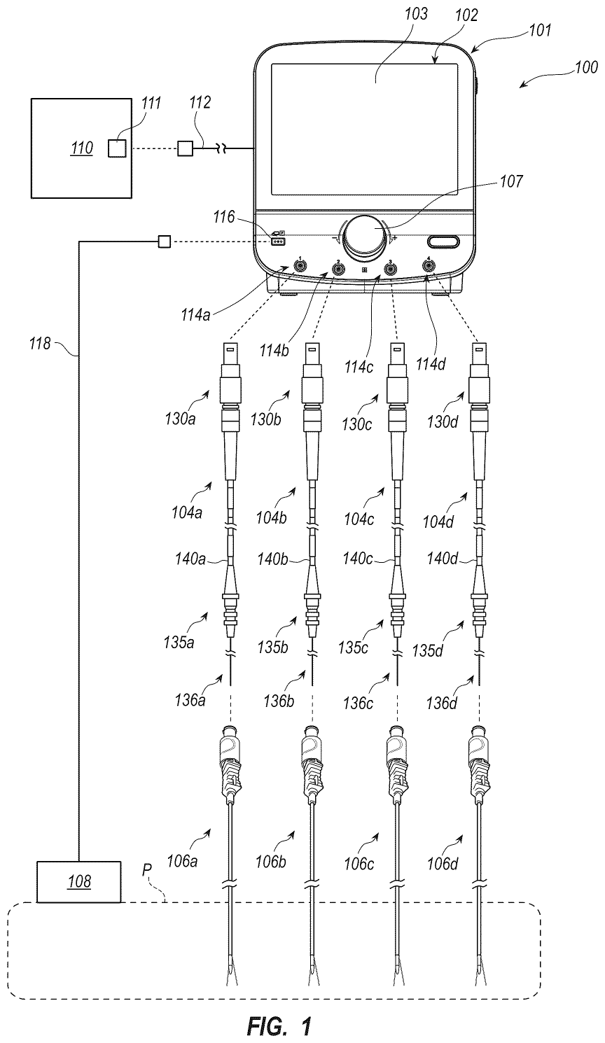

FIG. 1 depicts an embodiment of a system 100 that includes a control machine 101 and a plurality of instrument assemblies 104a-d that are operable or controllable by the control machine 101. In various embodiments, the illustrated system 100 can be described as a medical system or as an RF ablation system. In some embodiments, the system 100 is well-adapted for use in RF neurotomy, and may be referred to as an RF neurotomy system.

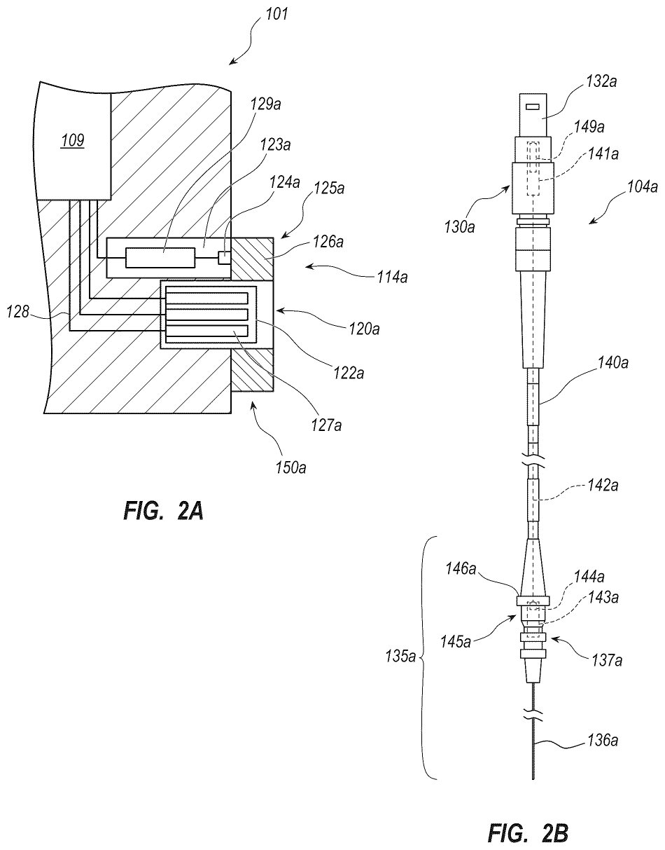

With reference to FIGS. 1 and 2B, in the illustrated embodiment, each of the instrument assemblies 104a-d may also or alternatively be referred to as a medical instrument assembly or as an RF probe assembly. Other instrument assemblies described herein likewise may also be referred to as medical instrument assemblies. Each instrument assembly 104a-d includes an RF probe 135a-d coupled to a distal end of a cable 140a-d, and a connector 130a-d is coupled to a proximal end of the cable 140a-d. Each RF probe 135a-d includes an active element or electrode 136a-d of the RF probe 135a-d (while in some instances, the electrode 136a-d may itself be referred to as a probe). As shown in FIG. 2B, the RF probe 135a (representative of the additional probes 135b-d) may include a hub 137a to which each of the electrode 136a and the cable 140a are attached. The cables 140a-d can include one or more communication lines (such as the communication line 142a) that electrically couple the connector 130a-d to the RF probe 135a-d of the respective instrument assembly 104a-d.

The RF probes 135a-d may more generally be referred to as medical instruments. That is, although the present discussion is provided in the context of RF probes, it is to be understood that different medical instruments may be present at the distal end of the cables 140a-d in other embodiments. That is, in other embodiments, the control machine 101 can be configured to control operation of other medical instruments. In the illustrated embodiment, the control machine 101 is configured to control delivery of RF energy to a patient in which the RF probes 135a-d are positioned.

With reference to FIG. 1, in some embodiments, each of the instrument assemblies 104a-d may be usable with an additional or further medical instrument 106a-d, which can be physically and electrically connectable with the RF probes 135a-d and can be considered as selectively attachable and/or selectively removable components of the respective instrument assemblies 104a-d. For example, in the illustrated embodiment, each medical instrument 106a-d comprises an RF neurotomy needle that is configured to receive therein the electrode 136a-d of a respective RF probe 135a-d, such that the RF probe 135a-d physically seats within the needle to conductively contact the needle and thereby electrically couple therewith. In various embodiments, the electrodes 136a-d can energize a needle tip, or a needle tip and/or deployable tines, of the medical instrument 106a-d. For example, in various embodiments, one or more of the medical instruments 106a-d can be or can resemble any of the embodiments of RF needles described in U.S. Pat. No. 10,716,618, titled SYSTEMS AND METHODS FOR TISSUE ABLATION, issued Jul. 21, 2020, and/or U.S. Pat. No. 10,736,688, titled METHODS AND SYSTEMS FOR RADIO FREQUENCY NEUROTOMY, issued Aug. 11, 2020, the entire contents of each of which are hereby incorporated by reference herein. In various embodiments, one or more of the medical instruments 106a-d can be a NIMBUS® Multi-tined Expandable Electrode, available from Stratus™ Medical, LLC, of Magnolia, Texas. In other or further embodiments, one or more of the medical instruments 106a-d can be a VESTA® RF Cannula, available from Stratus™ Medical, LLC, of Magnolia, Texas.

In some embodiments, the control machine 101 can operate one or more of the RF probes 135a-d and associated medical instruments 106a-d in a monopolar fashion or mode. For example, in some embodiments, a grounding or electrode pad 108 may be affixed to the skin of the patient P, and an electrical cable 118 extending from the grounding pad 108 can be coupled with a port 116 of the control machine 101. Per the nomenclature of the present application, the electrical cable 118 can include any suitable communication line for transmitting electrical energy between the grounding pad 108 and the control machine 101. For example, the communication line can include an electrical line or electrical lead.

For each of the one or more medical instruments 106a-d that will be operated in the monopolar mode, the connector 130a-d of the associated instrument assembly 104a-d can be coupled with the control machine 101. The one more medical instruments 106a-d can be inserted into the patient P (e.g., into proximity to one or more nerves targeted for ablation). Where applicable or desirable, tines of the medical instruments 106a-d can be deployed. At any suitable stage, such as before or after insertion of one or more of the medical instruments 106a-d or before or after deployment of the tines of a given medical instrument 106a-d, the medical instrument 106a-d can be coupled with its respective RF probe 135a-d. Stated otherwise, the RF probes 135a-d can be inserted into the respective medical instruments 106a-d. For each medical instrument 106a-d operated in the monopolar mode, an electrical circuit can include, using just “a”-valued components as an example, the control machine 101, the instrument assembly 104a, the medical instrument 106a, the patient P, the electrode pad 108, and the cable 118 back to the control machine 101. Each of the “b”-, “c”-, and “d”-valued components can form similar individual or separate monopolar circuits.

In some embodiments, the control machine 101 can operate two or more of the medical instruments 106a-d in a bipolar fashion. For example, in some embodiments, the control machine 101 can operate pairs of the medical instruments 106a-d (e.g., the medical instruments 106a,b) in bipolar fashion, in which one of the medical instruments 106a-d in a pair (e.g., the medical instrument 106a) serves as an active electrode, and the other medical instrument 106a-d in the pair (e.g., the medical instrument 106b) serves as a return electrode, at least briefly. In some instances, which of the two electrodes serves as the active electrode and which serves as the return electrode of a given bipolar pairing may alternate rapidly. In some illustrative examples, the pairs of medical instruments 106a,b and/or 106c,d may be operated in a bipolar mode, whether separately (e.g., the pair of medical instruments 106a,b followed by the pair of medical instruments 106c,d) or simultaneously (e.g., the pair of medical instruments 106a,b at the same time as the pair of medical instruments 106c,d), depending on positioning of the medical instruments 106a-d within the patient P. In other or further illustrative examples, a different pairing of the medical instruments 106a-d, such as the pair of medical instruments 106b,c and/or the pair of medical instruments 106a,d, may be operated separately or simultaneously, depending on positioning of the medical instruments 106a-d within the patient P. For each bipolar pairing of medical instruments 106a-d, an electrical circuit (using just “a”- and “b”-valued components as an example) can include the control machine 101, the instrument assembly 104a, the medical instrument 106a, the patient P, the medical instrument 106b, and the instrument assembly 104b back to the control machine 101. The “c”- and “d”-valued components, the “b”- and “c”-valued components, and the “a”- and “d”-valued components can form similar paired bipolar circuits. In certain embodiments, various bipolar pairings may be selectable via the control machine 101.

For example, in some instances, a practitioner may position each of the medical instruments 106a-d at different elevations of a spine of the patient P in the order shown. The practitioner may select, via the control machine 101, a bipolar pairing of the medical instruments 106a,b and a further bipolar pairing of the medical instruments 106c,d for a first ablation procedure. For example, RF energy may be applied simultaneously through each bipolar pairing during a first “burn” event, thereby forming a lesion within the patient P that extends at least between the electrodes of the medical instruments 106a,b and another lesion within the patient P that extends at least between the electrodes of the medical instruments 106c,d. Subsequently, a practitioner may select, via the control machine 101, a bipolar pairing of the medical instruments 106b,c for a second ablation procedure, which can proceed without changing the position of at least the medical instruments 106b,c or their associated instrument assemblies 104b,c. The practitioner may then proceed with a second “burn” event, in which RF energy is applied through the circuit that includes the medical instruments 106b,c, thereby forming a lesion within the patient P that extends at least between the electrodes of the medical instruments 106b,c.

In still other or further embodiments, the “a”- and “c”-valued components and/or the “b”- and “d”-valued components can form paired bipolar circuits, which may be selectable by a user via the control machine 101. An appropriateness of such pairings may depend upon relative positioning of the various medical instruments 106a-d within the patient P.

With continued reference to FIG. 1, the control machine 101 can be of any suitable variety, or stated otherwise, may be a medical control machine configured to control any of a variety of different types of medical instruments. For example, in the illustrated embodiment, the control machine 101 includes an RF generator configured to control RF probes. In some embodiments, the RF generator is configured to operate one or more RF probe/needle assemblies in a monopolar mode and/or two or more RF probe/needle assemblies in a bipolar mode, such as in manners described above. The control machine 101 may be said to control the medical instrument to perform a function for treatment of a patient. For example, the control machine 101 can control delivery of RF signals to the RF probes 135a-d. These RF signals can in turn be delivered through the medical instruments 106a-d into the patient to ablate a target region, which may include one or more nerves.

In certain embodiments, the control machine includes a display 102, which may include a screen 103. In some embodiments, the display 102 may also be used as an input device. For example, in some embodiments, the screen 103 comprises a touchscreen that can be manipulated by a user to interface with the control machine 101.

The control machine 101 can include any other suitable input and/or output devices. For example, in the illustrated embodiment, the control machine 101 includes a control knob 107. In various embodiments, the control knob 107 can be rotated to navigate through different options displayed on the screen 103 and/or can be depressed to make one or more selections. Other operations of the control knob 107 are also contemplated. Other suitable input devices that may, in some embodiments, be coupled with the control machine 101 include memory devices (e.g., USB drives), keyboards, mice, etc. In some embodiments, the control machine 101 includes one or more speakers for outputting sounds for auditory perception by a user.

In the illustrated embodiment, any suitable power source 110 can be coupled with the control machine 101. In the illustrated embodiment, the power source 110 comprises dedicated electrical wiring and an electrical outlet 111, to which can be coupled a power cord 112 of the control machine 101.

With continued reference to FIG. 1, the control machine 101 can include a plurality of ports 114a-d that can each be coupled individually with one of the instrument assemblies 104a-b. In some embodiments, the ports 114a-d can be used interchangeably, or stated otherwise, each of the instrument assemblies 104a-b can be coupled interchangeably with any of the ports 114a-d. For example, in some embodiments, a single instrument assembly 104a can be used with any of the ports 114a-d. In certain of such embodiments, the control machine 101 can operate the associated medical instrument 106a-d (e.g., the medical instrument 106a) in a monopolar mode equally well from any of the ports 114a-d. As a further example, in some embodiments, two instrument assemblies 104a-d (e.g., the instrument assemblies 104a,b) can be used with any two of the ports 114a-d. In certain of such embodiments, the control machine 101 can operate the associated medical instruments (e.g., the medial instruments 106a,b) either individually, in a monopolar mode, or as a pair, in a bipolar mode, equally well from any pairing of the ports 114a-d.

In certain embodiments, the control machine 101 and/or the ports 114a-d are specially configured for identifying which of the instrument assemblies 104a-d is connected to each of the ports 114a-d. For example, as further discussed below, in some embodiments, each of the instrument assemblies 104a-d includes identifying information (e.g., a model number) stored within the instrument assembly, which information can be accessed, obtained, read, or otherwise transferred to and used by the control machine 101 to implement one or more controls of the instrument assemblies and/or indicators associated with the instrument assemblies and/or associated with the ports, as further discussed below. In other or further embodiments, the instrument assemblies 104a-d can be configured to interface with the control machine 101 and/or the respective ports 114a-d to assist in identifying which port 114a-d each of the instrument assemblies 104a-d is connected to.

With reference to FIG. 2A, in some embodiments, each port 114a-d includes and/or is associated with componentry that enables or facilitates instrument identification. For convenience, only the port 114a is shown in cross-section, with certain components thereof depicted schematically. The port 114a is representative of the remaining ports 114b-d, each of which may include identical componentry.

The port 114a can include a connector 120a, which can include a connection interface 122a that is configured to physically connect with a connection interface 132a of a connector 130a of any of the instrument assemblies 104a-d (see FIG. 2B). For example, the connection interfaces 122a, 132a of the port 114a and the connector 130a, respectively, can be complementary to each other. In some instances, one of the connection interfaces 122a, 132a includes a plurality of electrical contacts or electrodes 127a, e.g., in the form of pins and/or sockets, and the other of the connection interfaces 122a, 132a includes a plurality of complementary electrical contacts or electrodes 127a, e.g., in the form of sockets and/or pins, that mate therewith. The connection interface 122a can include any suitable electrode 127a (e.g., socket, pin, and/or other electrical contact) arrangement to achieve electrical connection with the connection interface 132a of any of the instrument assemblies 104a-d. In some embodiments, the connection interface 122a includes a number of sockets and/or pins that may typically be used to power and/or control an RF probe and thermocouple (e.g. two sockets) and may include an additional number of sockets and/or pins (e.g., one, two, three, four, or five sockets) to provide supplemental power and/or controls to additional componentry associated with the instrument assembly 104a. In some instances, at least some of the supplemental power and/or controls can power and/or control an identification feature of the instrument assembly 104a-d.

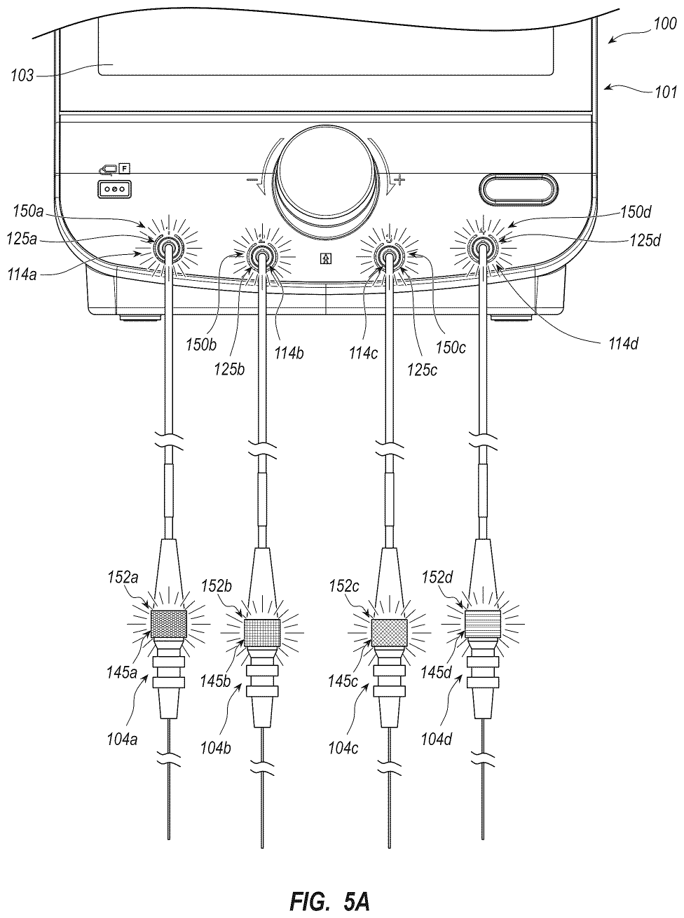

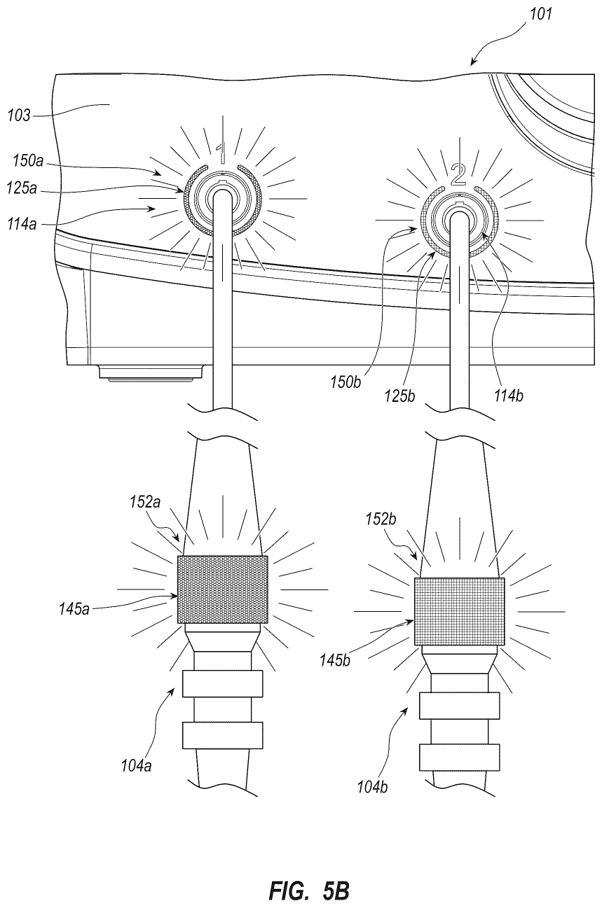

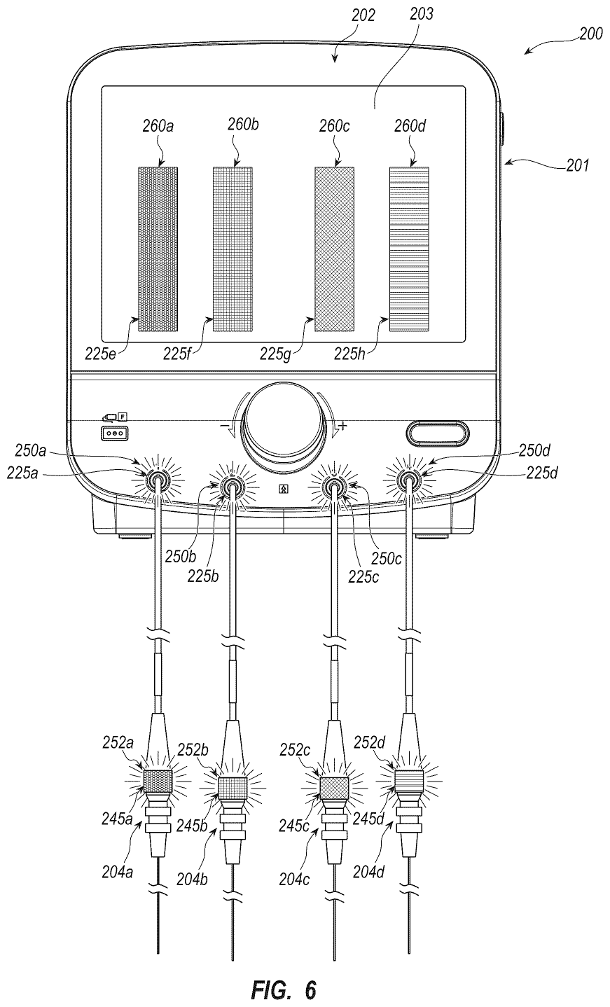

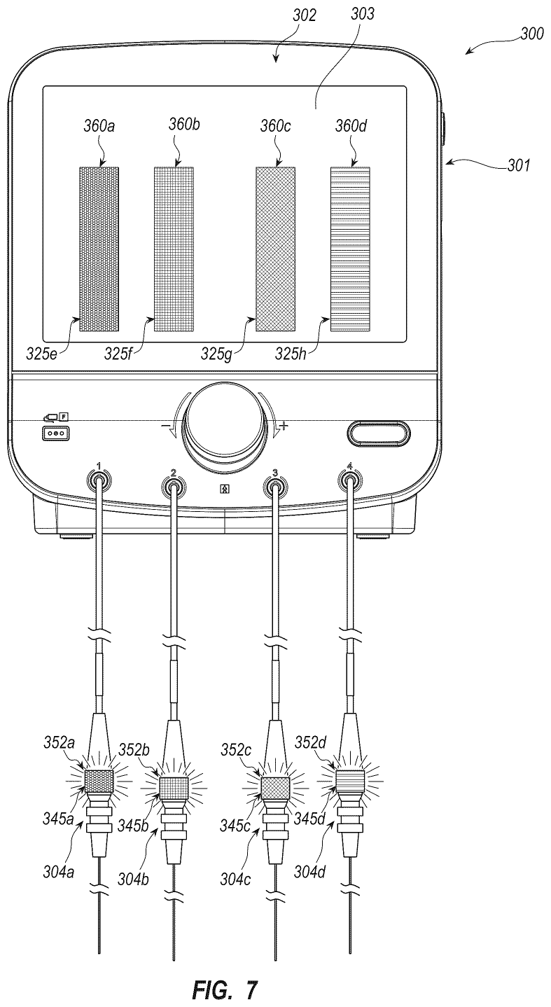

With continued reference to FIG. 2A, the port 114a can include an indicator 125a that is associated with the port 114a in any suitable manner. In the illustrated embodiment, the indicator 125a is physically associated with the port 114a. In particular, at least a portion of the indicator 125a is physically at or adjacent to the port 114a. The indicator 125a may also or alternatively be referred to as a port indicator, as the indicator 125a is associated with a particular port of the control machine 101. The indicator 125a may also or alternatively be referred to as a control machine indicator, as the control machine includes the indicator 125a. In the illustrated embodiment, the indicator 125a includes a lighting element, light pipe, light conduit, or light guide 126a that extends about a majority of or, in some embodiments, a full periphery of the port 114a (see also, e.g., FIG. 5B). In other or further embodiments, the indicator 125a may be physically associated with the port 114a in other manners, such as by being aligned with the port 114a in some manner, such as by vertical alignment (see, e.g., FIGS. 6-8). In still other or further embodiments, the indicator 125a may be associated with the port 114a in manners alternative or additional to physical proximity or physical alignment. For example, in various embodiments, the indicator 125a may display a visual indicium 150a, such as a color, a symbol (one or more alphanumerical characters, shapes, etc.), a light, a light pattern (e.g., a particular on/off sequence), a colored light, etc. that matches a color, symbol, state, pattern, etc. that is representative of the port 114a. In some embodiments, the visual indicium 150a may be displayed on the screen 103 (see, e.g., FIGS. 6 and 7).

With continued reference to FIG. 2A, in some embodiments, the visual indicium 150a is or includes a permanent or non-changeable element. For example, in some embodiments, the indicator 125a may include a ring (e.g., of molded plastic) in place of the light guide 126a and/or a different or further piece that is affixed to or near the port 114a. The visually discernable color of the ring can be or can contribute to the visual indicium 150a. In other or further embodiments, the indicator 125a may include a permanent symbol, such as an alphanumerical character (e.g., 1, 2, 3, 4, etc.) that is affixed to the control machine 101 at or near the port 114a, such as by being molded into the ring or other piece mentioned above, molded into or onto the body or housing of the control machine 101, printed or affixed onto the housing or control machine 101 (e.g., via ink, adhesive stickers, or the like), etc.

In other or further embodiments, the visual indicium 150a is or includes a non-permanent, selectable, dynamic, actuatable, activatable, controllable, changeable, or otherwise transiently displayable element. For example, in some embodiments, the indicator 125a may include the light guide 126a, which can be selectively illuminated by a light-emitting diode (LED) unit 124a. The LED unit 124a can include a single LED or an LED array or assembly. For example, in some embodiments, the LED unit 124a includes one or more multi-color RGB LED arrays. In various embodiments, the LED unit 124a can intrinsically display, or can be controlled to display, a predetermined color. In various embodiments, the LED unit 124a can be selectively lighted, such as, for example: for a predetermined period when the connector 130 of an instrument assembly 104a-d is first coupled to the connector 120a; throughout any period during which the connector 120a and a connector 130 of an instrument assembly 104a-d are connected, including for a full period during which one of the instrument assemblies 104a-d is attached to one of the ports 114a-d; for a full period during which any of the instrument assemblies 104a-d is attached to any of the ports 114a-d; and/or whenever a user chooses to illuminate the indicator 125a, such as by an actuator (e.g., virtual button) selected on a touch screen 103 and/or by a dedicated actuator (e.g., button or toggle switch) coupled to the housing of the control machine 101. As further discussed hereafter, in some embodiments, the LED unit 124a may be configured to permit selection of a color produced thereby.

With continued reference to FIG. 2A, the port 114a can include a circuit board 123a associated therewith that includes the LED unit 124a, which may include a controller for the LED or LED array (e.g., RGB LED unit). The circuit board 123a may also be referred to as a printed circuit board or PCB. In the illustrated embodiment, the printed circuit board 123a is physically positioned at the port 114 and oriented such that the LED unit 124a is optically coupled with the light guide 126a so as to illuminate the light guide 126a.

In further embodiments, the printed circuit board 123a further includes an RFID reader 129a. In other embodiments, the RFID reader 129a may be included on a separate circuit board, which may be positioned elsewhere in the control machine 101. In some instances, however, in may be advantageous for the RFID reader 129a to be positioned near the port 114a so as to facilitate communication between the RFID reader 129a and an RFID tag 149a (see FIG. 2B) associated with the instrument assembly 104a when the instrument assembly 104a is connected to the port 114a. For example, close proximity of the RFID reader 129a and the RFID tag 149a can permit or facilitate communication between these components and/or can require less power consumption to achieve the communication. In other or further instances, it may be advantageous to include both the RFID reader 129a and the LED unit 124a on the same printed circuit board, as such can permit compact assembly of the port 114, reduce production cost of the PCB 123a, and/or permit mass production of a single unit that can be used with any of the ports 114a-d.

The terms “RFID reader” and “RFID tag” are used herein in a broad sense and include the standard meanings of these terms. For example, an RFID reader can be configured to communicate with RFID tags to determine information associated with those tags. Further, embodiments of RFID readers can be configured to communicate information to RFID tags so as to store information in the RFID tags. Accordingly, the term “RFID reader” does not solely connote the ability to read RFID tags, but includes the ability to write to RFID tags. The term RFID reader/writer may also be used with respect to RFID readers.

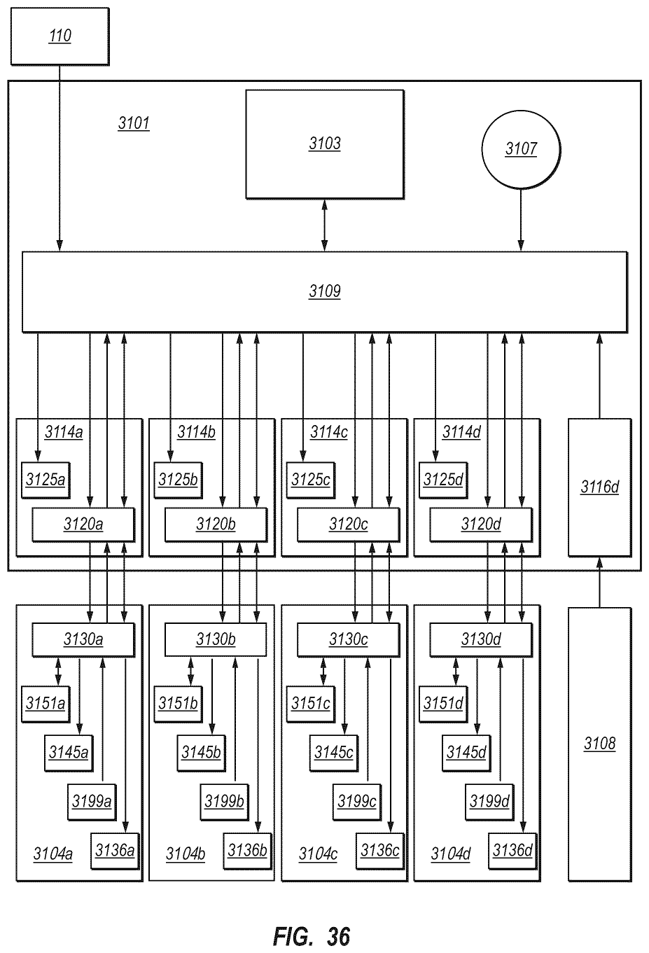

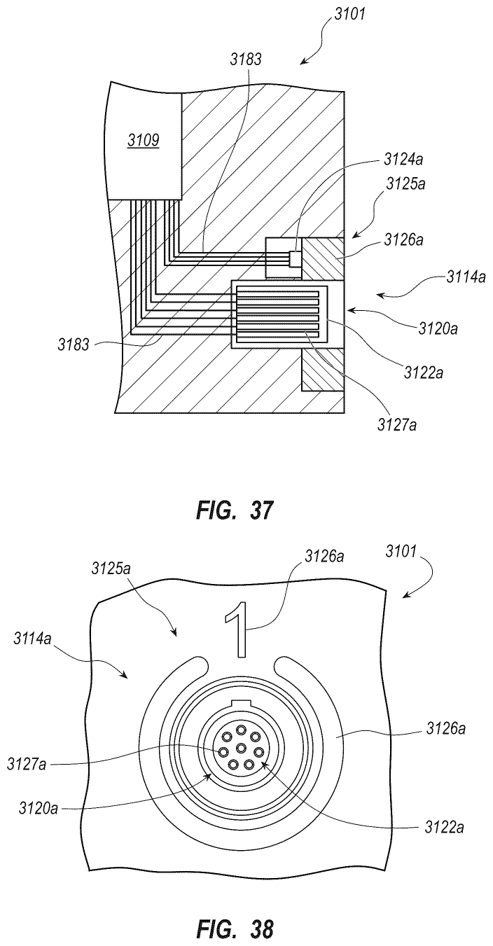

In certain embodiments, the PCB 123a is configured to communicate with a controller 109 of the control machine 101. The controller 109 can be configured to control operation of the control machine 101. For example, the controller 109 can resemble the controller 3109 discussed below with respect to FIGS. 36 and 37. The controller 109 can be implemented in any suitable combination of circuitry, hardware, and/or software, including one or more processors, microprocessors, controllers, microcontrollers, memory devices, logic devices, computers, etc. In some embodiments, the controller 109 can include a field-programmable gate array (FPGA), e.g., among other control circuitry. The controller 109 can include one or more memory devices that store machine readable instructions, which instructions can be read and implemented by one or more processors and/or other control circuitry of the controller 109. Indeed, the controller 109 can include any suitable architecture for controlling operation of the ports 114a-d, the instrument assemblies 104a-d, and certain components of each of these, including, for example, the indicators 125a-d of the ports 114a-d and indicators of the instrument assemblies 104a-d, which are described further below. The controller 109 can further be configured to control operation of the medical instruments 135a-d. For example, in various embodiments, the controller 109 may control the indicators 125a-d of the ports 114a-d and indicators of the instrument assemblies 104a-d while simultaneously controlling operation of the medical instruments 135a-d.

For example, in some embodiments, the controller 109 can be programmed, preprogrammed, or preconfigured to assign a predetermined color to each of the ports 114a-d. For example, in some embodiments, a predetermined color scheme may be programmed into one or more memory devices of the controller, such as in the form of machine-readable instructions. The controller 109 can be configured to read and implement these instructions, and responsive thereto, can deliver control signals to each of the PCBs 123 associated with the ports 114a-d to thereby control a color of light displayed by the indicators 125a-d.

In some embodiments, the controller 109 is configured to cause the indicators 125a-d of the ports 114a-d to each display a color that differs from the colors displayed by the remaining ports. Stated otherwise, in certain implementations, no two indicators 125a-d display the same color. Each color may uniquely identify one of the ports 114a-d.

In other embodiments, a user can select a color for one or more of the ports 114a-d. Stated otherwise, in some embodiments, the touchscreen 103 or other input device (e.g., the control knob 107) may be used to select a color for one or more of the indicators 125a-d. The color selection instructions can be provided to the controller 109. Responsive to these instructions, the controller 109 can then provide suitable control signals, such as, for example, pulse-width modulation (PWM) signals to the LED unit 124a. The LED unit 124a may include an array of RGB LEDs, the colored light from which can be mixed in appropriate amounts to achieve the user-selected color. That is, the controller 109 can provide control signals to the LED unit 124a that yield the user-selected color. In other or further embodiments, the user may select an illumination state of (e.g., off or on), an illumination duration, and/or an illumination pattern of the LED unit 124a and/or the illuminated light guide 126a, which selection can be processed by and/or communicated from the controller 109 to the circuit board 123a.

As previously discussed, in some instances each port 114a-d may have a predetermined or preselected color associated therewith, which preselection may be stored, for example, in nonvolatile memory of the controller 109. In other instances, a color of one or more of the ports 114a-d may be variable or dynamically selectable, such as by a user entering in a desired color via the touch screen 103. In certain embodiments of either scenario, the controller 109 can control the lighting of each port 114a-d and the lighting of instrument assemblies 104a-d coupled with any of the ports (as further discussed below). Further, in various embodiments of either scenario, it can be advantageous for the LED unit 124a to be configured to produce a variety of colors in response to directions provided by the controller 109. This can, for example, permit the same PCB 123a to be used in each of the ports 114a-d, which can be assigned different colors as previously discussed, and thus allow mass production of a single unit that can be used with any of the ports 114a-d with concomitant economies of scale.

With continued reference to FIG. 2A, illustrative communication lines 128 are shown. The communication lines 128 provide communication channels between various components of the control machine 101. For example, three communication lines 128 are depicted that provide a communication channel between the controller 109 and individual electrical contacts 127a of the port 114a. A further communication line 128 is depicted extending between the controller 109 and the printed circuit board 123a, or more specifically, the RFID reader 129a component on the PCB 123a. Further, an illustrative communication line is shown between the RFID reader 129a and the LED unit 124a.

It is to be understood that more communication lines 128 than are shown in FIG. 2A are possible. For example, in some instances, the LED unit 124a may include an RGB LED array that can be controlled directly by the controller 109 via three or more communication lines 128. Examples of such LED units and controls thereof are discussed below. Moreover, the schematic view of FIG. 2A omits further electrical contacts 127a that may be present in other embodiments.

As used herein, the term “communication” is to broadly interpreted. “Communication” includes the standard meanings of this term, and can include transfer or transmission of data, information, control signals, etc. This transfer may be by way of electrical communication, optical communication, etc. The term “communication” also includes delivery of electrical energy or power, which may not necessarily be for purposes of data transfer. For example, power lines and electrical leads may be capable of “communicating” electrical energy from one component to another. The term “communication” thus can refer to transport, delivery, and/or transfer generally. In like manner, the term “communication line” can include traditional understandings of this term, such as electrical lines or leads, optical fibers, etc. for delivering information, control signals, etc. The term “communication line” can also include power lines, electrical lines, or the like. The communication lines can provide channels of communication (including power transfer, etc.) between components coupled by those communication lines. Thus, specific references herein to communication lines, electrical lines, or the like may all be understood as more generally referring to communication lines that provide communication channels or pathways among the components.

With reference to FIG. 2B, in certain embodiments, the instrument assembly 104a (which can be representative of the remaining instrument assemblies 104b-d), includes the connector 130a at a proximal end of the cable 140, and the probe 135a at a distal end of the cable 140. As previously discussed, the connector 130a is couplable with any of the ports 114a-d.

In the illustrated embodiment, the connector 130a (e.g., a hub portion and/or a strain-relief portion of the connector 130a) includes a printed circuit board 141a, on which is included an RFID tag 149a. The PCB 141a is communicatively coupled with a further printed circuit board 143a positioned within the hub 137a of the probe 135a. For example, the PCBs 141a, 143a may be coupled via any suitable communication line 142a, such as one or more electrical wires, optical fibers, etc. The PCB 143a can include any suitable LED unit 144a, such as an RGB LED array. The LED unit 144a can desirably be configured to produce any color assigned thereto via the RFID tag 149a. In some embodiments, the LED unit 144a is optically coupled with a light guide 146a. In the illustrated embodiment, the light guide 146a is formed as a collar that encompasses a portion of the hub 137a. Other suitable arrangements, including other or further optical medial for transport, diffusion, display, or other treatment of light are contemplated.

With continued reference to FIG. 2B, the RF probe 135a can include an indicator 145a that is associated with the RF probe 135a. In the illustrated embodiment, the indicator 145a is physically associated with the RF probe 135a. In particular, the indicator 145a is fixedly secured to the RF probe 135a, and further, is positioned at least partially on and/or at least partially within the hub 137a of the probe 135a. The indicator 145 may also or alternatively be referred to as a probe indicator 145a, as the indicator 145a is used in identifying the probe 135a or, stated otherwise, is used in distinguishing the probe 135a from other probes. The indicator 145 may also or alternatively be referred to as an assembly indicator 145a, as the instrument assembly 104a includes the indicator 145a.

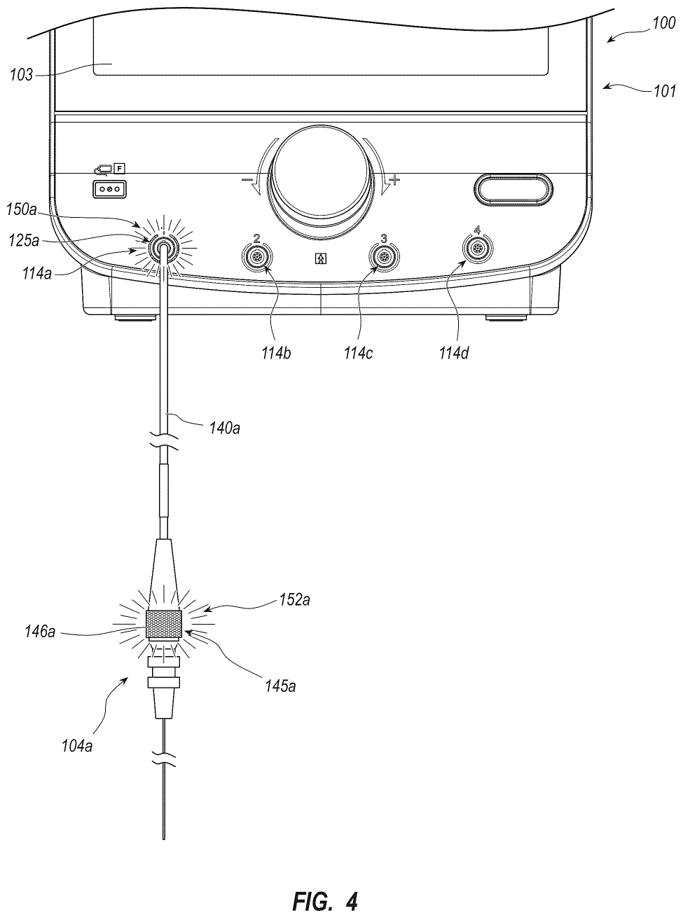

The indicator 145a may display a visual indicium 152a (see FIG. 4), such as a light of a constant or unchanging color in the illustrated embodiment. The visual indicium 152a can be a non-permanent, selectable, dynamic, actuatable, activatable, controllable, or otherwise transiently displayable element. For example, in some embodiments, the indicator 145a may include the light guide 146a, which can be selectively illuminated by the LED unit 144a. The indicator 145a can be configured to receive information or control signals from the port 114a so as to display the visual indicium 152a in a manner that associates the visual indicium 152a of the probe 135a with the visual indicium 150a of the port 114. Stated otherwise, the indicator 145a can be responsive to information from and/or regarding the specific port 114a-d with which the instrument assembly 104a is connected in order to readily identify to a user the association of the probe 135a with that specific port 114a-d. In some instances, it can be advantageous for the indicator 145a to be positioned on or near the probe 135a itself, as this obviates other more cumbersome methods of determining an association between a specific port 114a-d and the probe 135a, such as, for example, visually, physically, or otherwise following a path of the cable 140a between the connected port 114a-d and the probe 135a.

With reference to FIGS. 2A, 3A, and 4, in use, the instrument assembly 104a can be coupled with any of the ports 114a-d. In the scenario depicted in FIG. 4, the connector 130a of the instrument assembly 104a is coupled with the port 114a. The RFID tag 149a can be configured to receive one or more signals from the RFID reader 129a of the port 114a when the connector 130a is coupled to the port 114a in this manner. The one or more signals from the RFID reader 129a can provide information and/or control signals regarding the color to be displayed via the LED unit 144a. For example, the color information can associate the color displayed via the indicator 125a of the port 114a with the color displayed via the indicator 145a of the RF probe 135a. For example, the displayed colors can match one another. Color information can be delivered to and/or accessed by an LED controller that is in communication with the RFID tag 149a. The LED controller can, for example, be included on one or more of the PCBs 141a, 143a. The LED controller can cause the LED unit 144a, and hence the light guide 146a, to display the prescribed color.

In some embodiments, power is provided to the PCB 141a and/or the PCB 143a of the instrument assembly 104a via one or more electrical leads, which can be coupled with one or more of the additional electrodes (e.g., sockets and/or pins) in the connection interface 132a of the port 114a, as previously discussed. In some embodiments, one or more communication leads may extend from the connection interface 132a of the connector 130a, through the cable 140a, and to the PCB 143a.

In some embodiments, the PCB 141a of the instrument assembly 104a includes a memory device. For example, the RFID tag 149a can store information that is retrievable by the RFID reader 129a of the control machine 101. In some embodiments, this stored information can be indicative of the presence of the indicator 145a in the instrument assembly 104a. For example, in some embodiments, the stored information can specify a model number of the instrument assembly 104a or any other suitable data that can be used by the RFID reader 129a and/or the controller 109 to determine that the instrument assembly 104a is configured to display colored light via the indicator 145a. In other or further embodiments, the PCB 141a can include other or further memory devices of any suitable variety.

In some embodiments, when the connector 130a of the instrument assembly 104a is in close proximity to the port 114a and/or is connected to the port 114a, the RFID reader 129a can access the information indicative of the presence of the indicator 145a that is stored in the RFID tag 149a and/or in some other memory device on the PCB 141a of the instrument assembly 104a. In various embodiments, the RFID reader 129a can provide instructions or control signals to the RFID tag, which can be delivered to the PCB 143a and/or the LED unit 144a to thereby control light delivery via the indicator 145a. Note that the RFID reader 129a may be viewed as an extension of the controller 109, such that in instances where the RFID reader 129a may be able to independently control the indicator 145a, it may nevertheless be said that the controller 109 controls the indicator 145a.

In other or further embodiments, the controller 109 may obtain the stored information regarding the indicator 145a from the memory device of the instrument assembly (e.g., from the RFID tag 149a) via the RFID reader 129a of the port 114a. The controller 109 may direct the RFID reader 129a to obtain the information. In other or further instances, the RFID reader 129a may be configured to automatically obtain the information. In either instance, the controller 109 may access the obtained information from the RFID reader 129 and/or from the PCB 129. Based on the information thus obtained, the controller 109 can determine the presence of the indicator 145a. For example, in some embodiments, the controller 109 is configured to compare the information against stored data to make the determination. By way of illustrative example, the controller 109 may compare the obtained information, which may include information regarding a model number or some other identifying information regarding the instrument assembly 104a, against a table of acceptable values for this information. Any other suitable method of determining the presence of the indicator 145a is contemplated.

In some embodiments, in response to the determination, the controller 109 can control operation of the indicator 145a via the RFID reader 129a. For example, in some instances, the controller 109 can cause the indicator 145a of the instrument assembly 104a to display light of a first color, and can cause the indicator 125a of the port 114a to display light of the first color. In further instances, the controller 109 can cause the remaining ports 114b-d to display light of second, third, and fourth colors, respectively. In some instances, each of the first, second, third, and fourth colors differs from each of the remaining three colors. Further operations of light control via a control machine are discussed below, which operations can be performed via embodiments of the control machine 101 and embodiments of the system 100.

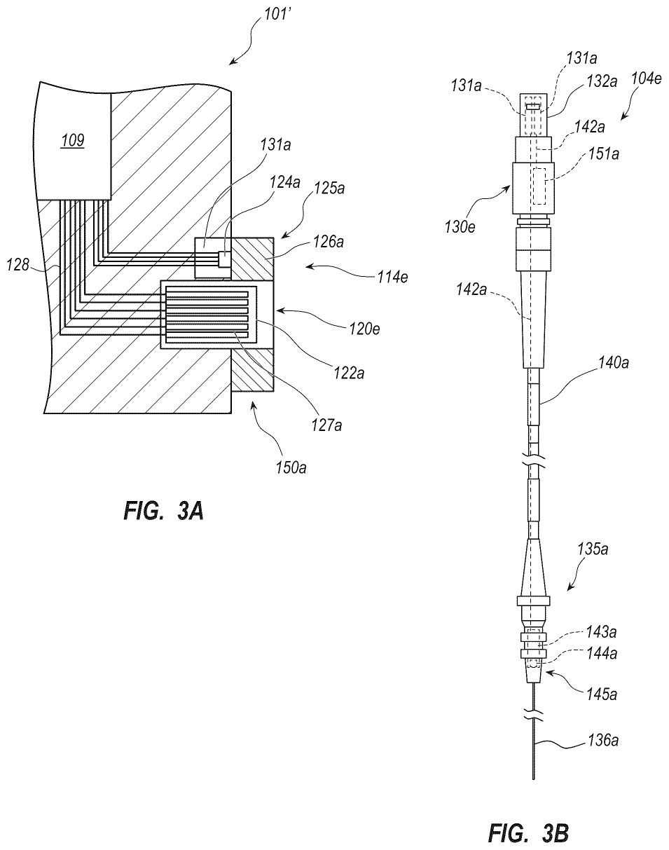

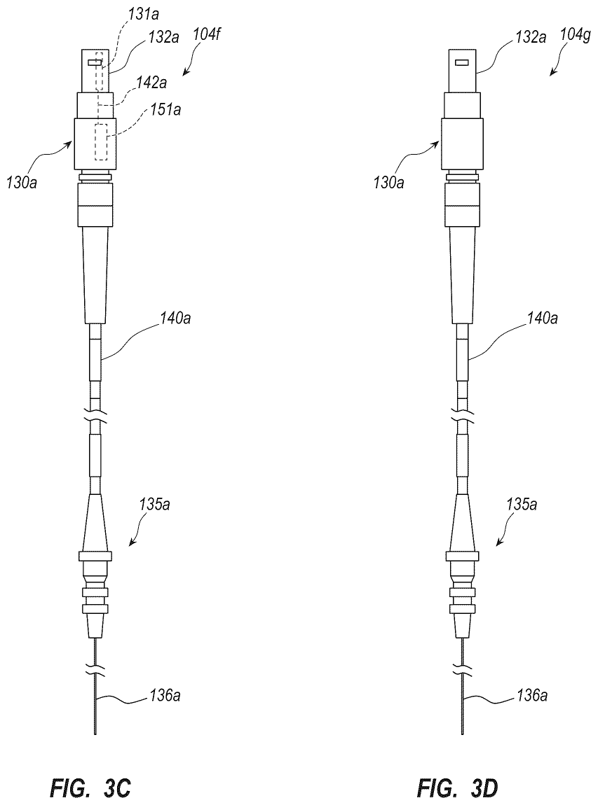

FIGS. 3A-3D depict another embodiment of a port 114e that is compatible with a control machine 101′ and embodiments of instrument assemblies 104e, 104f, 104g, respectively. The control machine 101′ and the instrument assemblies 104e, 104f, 104g can resemble the control machine 101 and the instrument assembly 104a discussed above in many respects. Indeed, the control machine 101′ can include many of the same or substantially the same components as the control machine 101, such that like items are identified with like numerals. Likewise, the instrument assemblies 104e, 104f, 104g can include many of the same or substantially the same components as the instrument assembly 104a, such that like items are identified with like numerals. The foregoing discussion of such like-identified components thus may not be repeated hereafter, and the foregoing discussion applies equally to these components of the control machine 101′ and the instrument assembly 104e.

With reference to FIGS. 3A and 3B, rather than including a wireless RFID system, the port 114e and the instrument assembly 104e include wired communication to the controller 109. For example, the port 114e includes a PCB 131a that includes an LED unit 124a, but does not, in the illustrated embodiment, include an RFID reader. The controller 109 can be coupled to the LED unit 124a via a plurality of communication lines 128. In some embodiments, three of the communication lines 128 are configured to provide control signal to three separate RGB diodes or RGB diode arrays, with each communication line 128 being dedicated to the control of a separate one of the RGB colors.

The connector 120e of the port 114e can include additional electrical contacts 127a, which are communicatively coupled with the controller 109 via further communication lines 128. The connector 130e of the instrument assembly 104e can include electrical contacts 131a that are configured to physically engage and electrically couple with electrical contacts 127a. The electrical contacts 131a of the connector 130e can be in communication with the printed circuit board 143a and/or the LED unit 144a via one or more communication lines 142a. For example, in some embodiments, the LED unit 144a is operational when coupled to a power source, and individual RGB colored LEDs are controllable via separate signals provided via separate electrical leads. The signals may be pulse-width modulation signals, for example. Accordingly, in some embodiments, the LED unit 144a functions via four communication lines 140a that extend from four separate electrical contacts 131a of the connector 130e. These electrical contacts 131a may join with four corresponding electrical contacts 127a of the connector 120e at the port 114e. The controller 109 may provide power to the LED unit 144a via a communication line 128 that is coupled with one of these four electrical contacts 127a, and may provide three separate streams or channels of control signals to control a color of light displayed by the LED unit 144a via three additional communication lines 128 that are coupled with the remaining three of the four electrical contacts 127a.

The control machine 101 and the instrument assembly 104e may include further communication lines 128, 142a via which the controller 109 can control operation of the RF probe 135a. For example, at least one additional communication line 142a may be coupled with each of the RF electrode 136a at a distal end of the instrument assembly 104e and a further electrical contact 131a at a proximal end of the instrument assembly 104e.

In some embodiments, the instrument assembly 104e includes a memory device 151a. The memory device 151a may be of any suitable variety, and may include a non-volatile memory storage medium. In some embodiments, the memory device 151a includes a read only storage medium. In other or further embodiments, the memory device 151a includes a read/write storage medium, which the controller 109 may be configured to write information to. In various embodiments, the memory device 151a includes an electrically erasable programmable read-only memory (EEPROM) device. For example, in some embodiments, the memory device 151a includes a single-wire EEPROM, such that, in various embodiments, only one or two communication lines 128 coupled with the controller 109 and a corresponding only one or two communication lines 142a coupled between the memory device 151a (e.g., the single-wire EEPROM) and one or two respective electrical contacts 131a are used for the controller 109 to read information from and/or write information to the EEPROM device. Other suitable memory devices are contemplated.

In some embodiments, the memory device 151a stores information regarding the instrument assembly 104e, such as information from which it can be determined that the instrument assembly 104e includes an indicator 145a, or more specifically, includes a controllable RBG LED array. In some embodiments, the stored information can include lock-out information, which the controller 109 can use to prevent use of unauthorized instrument assemblies. In some embodiments, the stored information includes a number of uses of the instrument assembly 104e. In various embodiments, the controller 109 may be configured to update one or more of the foregoing categories of information.

In certain embodiments, the controller 109 may obtain stored information regarding the indicator 145a from the memory device 151a of the instrument assembly, e.g., via a communication line 128 dedicated to communication with the memory device 151a. The controller 109 may access the stored information in accordance with any suitable protocol. Based on the information thus obtained, the controller 109 can determine the presence of the indicator 145a. For example, in some embodiments, the controller 109 is configured to compare the information against stored data to make the determination. By way of illustrative example, the controller 109 may compare the obtained information, which may include information regarding a model number or some other identifying information regarding the instrument assembly 104e, against a table of acceptable values for this information. Any other suitable method of determining the presence of the indicator 145a is contemplated.

In some embodiments, in response to the determination, the controller 109 can control operation of the indicator 145a, e.g., via three or four communication lines 128, such as previously discussed. For example, in some instances, the controller 109 can cause the indicator 145a of the instrument assembly 104e to display light of a first color, and can cause the indicator 125a of the port 114a to display light of the first color. In further instances, the controller 109 can cause remaining ports (e.g., such as the ports 114b-d depicted in FIG. 1) to display light of second, third, and fourth colors, respectively. In some instances, each of the first, second, third, and fourth colors differs from each of the remaining three colors. Further operations of light control via a control machine are discussed below, which operations can be performed via embodiments of the control machine 101′ and other or further embodiments of the system 100.

With reference to FIG. 3C, the instrument assembly 104f does not include an indicator. More specifically, the instrument assembly 104f does not include a controllable indicator, such as an indicator that includes a light source. The instrument assembly 104f does, however, include a memory device 151a such as previously discussed. In some embodiments, the memory device 151a includes stored information from which it can be determined (e.g., by the controller 109, when the instrument assembly 104f is coupled with the port 114e), that the instrument assembly 104f is devoid of any indicator.

In some embodiments, the controller 109 can cause the indicators 125a-d of the ports 114a-d (or other ports configured such as the port 114e), to display the same color of light (e.g., white light) when no instrument assemblies are attached to any of the ports and/or when no instrument assemblies that include indicators (e.g., such as any of the indicators 145a-d) are attached to any of the ports. Thus, for example, the controller 109 can cause all indicators 125a-d of the ports 114a-d to display the same color as each other prior to coupling of any instrument assemblies to the ports, after the decoupling of all instrument assemblies to the ports, and/or during a period when the only instrument assembly or instrument assemblies that are attached to the ports are devoid of indicators 125a-d.

With reference to FIG. 3D, the instrument assembly 104g likewise does not include an indicator, and more specifically, does not include a controllable indicator, such as an indicator that includes a light source. Further, the instrument assembly 104g is devoid of any memory device, such as, e.g., the memory device 151a previously discussed. In some embodiments, the controller 109 is able to determine that the instrument assembly 104g is devoid of an indicator even without accessing stored information from the instrument assembly 104g.

In some embodiments, the controller 109 can cause the indicators 125a-d of the ports 114a-d (or other ports configured such as the port 114e), to display the same color of light (e.g., white light) when no instrument assemblies are attached to any of the ports and/or when no instrument assemblies that include indicators (e.g., such as the instrument assemblies 104f, 104g) are attached to any of the ports. Thus, for example, the controller 109 can cause all indicators 125a-d of the ports 114a-d to display the same color as each other prior to coupling of any instrument assemblies to the ports, after the decoupling of all instrument assemblies to the ports, and/or during a period when the only instrument assembly or instrument assemblies that are attached to the ports are devoid of indicators 125a-d (such one or more of the instrument assemblies 104f, 104g).

In some embodiments, the controller 109 refrains from delivering light control signals to those electrical contacts 127 dedicated to delivering light control signals for the control of an instrument assembly indicator when a non-indicator instrument assembly is attached to the port that includes those electrical contacts. In other embodiments, the controller 109 may deliver or continue to deliver light control signals to such electrical contacts 127, even if the instrument assembly is devoid of an indicator, and in other or further instances, even after the controller 109 has determined that the instrument assembly is devoid of an indicator.

With reference to FIGS. 3A-3D, in various embodiments, the control machines 101, 101′ can control operation of the RF probes 135a, even when simultaneously controlling operation of the indicators, where applicable.

With reference to FIGS. 5A and 5B, the system 100, as previously discussed with reference to FIGS. 1, 2A, 2B, and 4, is shown in an operational state in which each of the four instrument assemblies 104a-d is coupled with a respective one of the ports 114a-d of the control machine 101. As shown, the indicators 125a-d associated with each of the ports 114a-d and the indicators 145a-d associated with each of the instrument assemblies 104a-d, respectively, convey information indicating correspondence. In particular, the indicia 150a-d are four distinct colors, one for each port 114a-d, and the indicia 152a-d are likewise the same four distinct colors, one for each instrument assembly 104a-d that matches the color of the respective port 114a-d to which the instrument assembly is coupled.

FIG. 6 depicts another embodiment of a system 200, which can resemble other systems disclosed herein, such as, for example, the system 100. Accordingly, features of the system 200 are designated with reference numerals similar to those of the system 100, with the leading digit “1” being replaced with “2.” Relevant disclosure set forth above regarding similarly identified features thus may not be repeated hereafter. Moreover, specific features of the system 200 may not be shown or identified by a reference numeral in the drawings or specifically discussed in the written description that follows. However, such features may clearly be the same, or substantially the same, as features depicted in other embodiments and/or described with respect to such embodiments. Accordingly, the relevant descriptions of such features apply equally to the features of the system 200. Any suitable combination of the features and variations of the same described with respect to the system 100 can be employed with the system 200, and vice versa. That is, disclosures regarding the systems 100, 200 can be appropriately applied to each other and/or to other systems described herein, in the interest of streamlining the present discussion. This pattern of disclosure applies equally to further embodiments depicted in the various figures and described herein, wherein the leading digits may be further altered or incremented.

FIG. 6 is an elevation view of another embodiment of a system 200 that includes a control machine 201 having a display 202, which can include a screen 203. The control machine 201 can further include four instrument ports and four instrument assemblies 204a-d that can be coupled interchangeably with any of the ports. Two separate indicators are associated with each of the four ports of the control machine 201. In particular, the control machine 201 includes a first set of indicators 225a-d which resemble the like-numbered indicators 125a-d. Further, the control machine 201 includes a second set of indicators 225e-h that are associated with each of the ports. In the illustrated embodiment, the indicators 225e-h are provided as display elements on the screen 303, and are physically associated with each of the ports by way of vertical alignment therewith. The indicators 225e-h are further logically associated with each of the ports by way of matching color schemes. The matching color schemes are represented by the colored indicia 250a-d of the ports and the matching colored indicia 260a-d displayed on the screen 203, respectively. This color scheme can further be matched by the colored indicia 252a-d produced by the indicators 245a-d of the instrument assemblies 204a-d.

FIG. 7 is an elevation view of another embodiment of a system 300 that includes a control machine 301 having a display 302, which can include a screen 303. The control machine 301 can further include four instrument ports and four instrument assemblies 304a-d that can be coupled interchangeably with any of the ports. In this embodiment, only a single indicator is associated with each of the four ports of the control machine 301. In particular, the control machine 301 only includes indicators 325e-h, which resemble the like-numbered indicators 225e-h. The indicators 325e-h are physically associated with each of the ports by way of vertical alignment. Each of the indicators 325e-h includes colored indicia 360a-d, which match colored indicia 352a-d produced by indicators 345a-d of the instrument assemblies 304a-d for purposes of correlating each instrument assembly 304a-d with the respective port with which it is coupled.

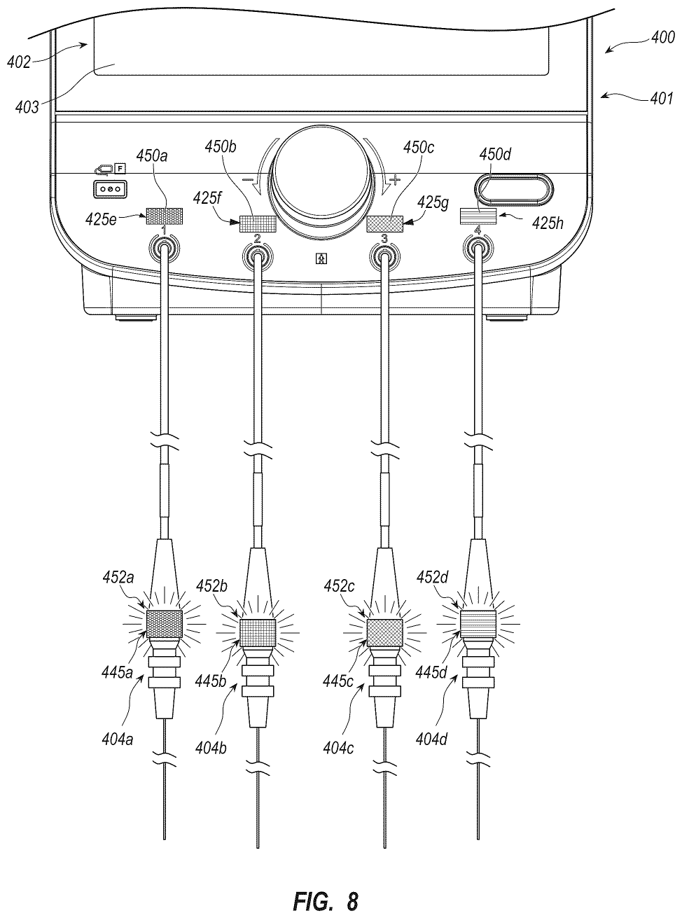

FIG. 8 is an elevation view of another embodiment of a system 400 that includes a control machine 401. The control machine 401 can further include four instrument ports and four instrument assemblies 404a-d that can be coupled interchangeably with any of the ports. In this embodiment, only a single indicator is associated with each of the four ports of the control machine 401. In particular, the control machine 401 only includes indicators 425e-h. The indicators 425a-d are physically associated with each of the ports by way of close proximity and vertical alignment.

In some embodiments, one or more of the indicators 425e-f provide indicia 450a-d that can be permanent in nature (e.g., such as previously discussed)—for example, the indicia 450a-d can be symbols, color elements, etc. that are physically attached to the control machine 401. In other embodiments, the indicia 450a-d may be more transitory in nature, such as may be displayed on small, dedicated screens.

The instrument assemblies 404a-d include indicators 445a-d that are configured to provide transient indicia 452a-d that can change to correspond with the respective port to which the instrument assembly is connected. For example, in some embodiments, the indicators 445a-d may comprise small dedicated screens that may display transient indicia 452a-d that correlates with the indicia 450a-d (whether permanent or transient) of the control machine 401.

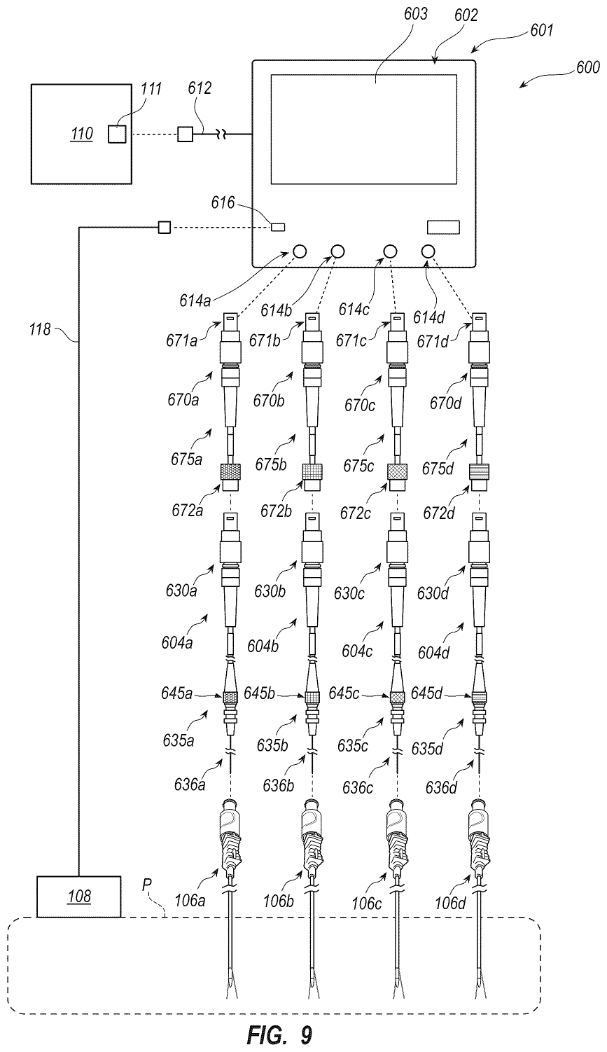

FIG. 9 depicts another embodiment of a system 600 that includes a control machine 601 and a plurality of instrument assemblies 604a-d that are operable or controllable by the control machine 601. In various embodiments, the illustrated system 600 can be described as a medical system or as an RF ablation system. In some embodiments, the system 600 is well-adapted for use in RF neurotomy, and may be referred to as an RF neurotomy system.

The system 600 can resemble the system 100 previously described. As with the control machine 101, the control machine 601 can include a plurality of ports 614a-614d to which the plurality of instrument assemblies 604a-d can be individually coupled. In some embodiments, each of the instrument assemblies 604a-d comprises an RF probe 635a-d having an RF electrode 636a-d at a distal end thereof, such as described above with respect to the instrument assemblies 104a-d.

Each of the instrument assemblies 604a-d can include one or more respective indicators 645a-d, which can resemble any of the indicators 145a-d described above. For example, the indicators 645a-d can serve to identify which instrument assembly 604a-d is coupled with a particular port 614a-d of the control machine 601. In some embodiments, however, the control machine 601 may not be configured to control the indicators 645a-d.

The system 600 can include one or more adapters 670a-d that are configured to control the indicators 645a-d of the instrument assemblies 604a-d, as further discussed below. For example, in some embodiments, one or more of the instrument assemblies 604a-d can be identical to embodiments of the instrument assemblies described above, including the embodiments depicted in FIGS. 1, 2B, 3B-3D. In some embodiments, the instrument assemblies 604a-d include RF tags by which an associated indicator 645a-d can be controlled. In certain of such instances, the adapters 670a-d usable with such instrument assemblies 604a-d can include RF readers for communicating with the RF tags. The adapters 670a-d and the instrument assemblies 604a-d thus may operate in manners such as previously described with respect to embodiments of the ports 114a-d and the instrument assemblies 104a-d, with the adapters 670a-d including componentry and functioning in manners cush as the ports 114a-d. In other embodiments, the instrument assemblies 604a-d include sufficient control circuitry and, in further instances, the ability to draw power from the control machine 601 and/or an internal power source so as to be able to perform the same and/or other functions as the controller 109 does in controlling light output of the indicators 645a-d. These and/or other configurations of the adapters 670a-d and the instrument assemblies 604a-d are contemplated, as further discussed below.

The adapters 670a-d can individually couple to any of the ports 614a-d of the control machine 601. In turn, the instrument assemblies 604a-d can individually couple to any of the adapters 670a-d. In the illustrated embodiment, each adapter 670a-d includes a proximal connector 671a-d by which the adapter is coupled to a respective port 614a-d of the control machine, and further includes a distal connector 672a-d to which a respective connector 630a-d of the instrument assemblies 604a-d is coupled. In certain embodiments, each adapter 670a-d includes one or more indicators 675a-d. As further discussed below, the indicators 675a-d can match or otherwise be associated with the indicators 645a-d of the respective instrument assemblies 604a-d, which can assist a user in readily identifying the port 614a-d to which the instrument assembly 604a-d is coupled, by way of the respective intermediary adapter 670a-d to which it is directly connected.

With continued reference to FIG. 9, in some embodiments, each of the instrument assemblies 604a-d may be usable with a further medical instrument 106a-d, which can be couplable with the RF probes 635a-d and can be considered as selectively attachable and/or selectively removable components of the respective instrument assemblies 604a-d. For example, in the illustrated embodiment, each medical instrument 106a-d comprises an RF neurotomy needle that is configured to receive therein the electrode 636a-d of a respective RF probe 635a-d. In various embodiments, the electrodes 636a-d can energize a needle tip, or a needle tip and/or deployable tines, of the medical instrument 106a-d, in manners such as described above.

In some embodiments, the control machine 601 can operate one or more of the instrument assemblies 604a-d and the medical instruments 106a-d attached thereto in a monopolar fashion or mode in manners such as previously discussed with respect to embodiments of the control machine 101. For example, in some embodiments, the grounding or electrode pad 108 may be affixed to the skin of the patient P, and the electrical cable 118 extending from the grounding pad 108 can be coupled with a port 616 of the control machine 601. Monopolar ablations may proceed in manners such as previously discussed.

In various embodiments, the control machine 601 can operate two or more of the medical instruments 106a-d in a bipolar fashion. For example, in some embodiments, the control machine 601 can operate pairs of the medical instruments 106a-d in manners such as previously discussed with respect to the control machine 101.

With continued reference to FIG. 9, the control machine 601 can be of any suitable variety. For example, in the illustrated embodiment, the control machine 601 includes an RF generator. In some embodiments, the RF generator is configured to operate one or more RF probe/needle assemblies in a monopolar mode and/or two or more RF probe/needle assemblies in a bipolar mode, such as in manners described above. In certain embodiments, the control machine includes a display 602, which may include a screen 603. In some embodiments, the display 602 may also be used as an input device. For example, in some embodiments, the screen 603 is a touchscreen that can be manipulated by a user to interface with the control machine 601.

In the illustrated embodiment, any suitable power source 110 can be coupled with the control machine 601. In the illustrated embodiment, the power source 110 comprises dedicated electrical wiring and an electrical outlet 111, to which can be coupled a power cord 612 of the control machine 601.

As previously mentioned, in various embodiments, the control machine 601 may not be configured to control the indicators 645a-d of the instrument assemblies 604a-d. For example, in some embodiments, the control machine 601 may be any of a variety of known RF generators that do not have the capability of controlling indicators of instrument assemblies. Illustrative examples of such RF generators include, without limitation: the MultiGen 2 Radiofrequency Generator, available from Stryker; the IonicRFT Generator, available from Abbott; the COOIEF RF System, available from Avanos; the Accurian™ Radiofrequency Ablation Platform, available from Medtronic; the G4™ RF Generator, available from Boston Scientific; and the OWL® Universal Radiofrequency Lesion Generator, available from Diros Technology, Inc.

As further described below, in various embodiments, the adapters 670a-d can draw power from one or more electrical contacts or electrodes (e.g., sockets) of the ports 614a-d in order to power the indicators 675a-d of the adapters 670a-d and/or in order to power and/or control the indicators 645a-d of the instrument assemblies 604a-d. In other or further embodiments, the adapters 670a-d may include a dedicated power supply for such operations. In some instances, one or more of the adapters 670a-d may not draw any power for such operations from the control machine 601.

In some instances, a variety of different adapters 670 may be used with different types of control machines 601. For example, some adapters 670 may be specifically configured for use with one type of control machine 601, further adapters 670 may be specifically configured for use with another type of control machine 601, and still further adapters 670 may be specifically configured for use with yet another type of control machine 601. For example, the various different control machines 601 may have different types of ports 614. In some instances, the ports 614 may have specific configurations of electrical contact pins or sockets. The ports 614 of one variety of control machine 601 may have more or fewer pins and/or pins arranged in different positions, as compared with the ports 614 of other control machines 601. In some instances, a port 614 may have a first set of electrical contacts that is electrically engaged with componentry of the control machine 601, such as for the delivery of electrical energy and/or signals thereby, and may further include a second set of electrical contacts that are not so engaged. The second set may be extra electrical contacts or dummy contacts.

The different adapters 670 may have different types of proximal connectors 671 so as to be usable with different types of ports 614 of different types of control machines 601. In some embodiments, the distal connectors 672 of the various types of adapters 670, however, may all be alike. For example, in some embodiments, any of the various types of adapters 670 may be used with the same type of instrument assembly 604.

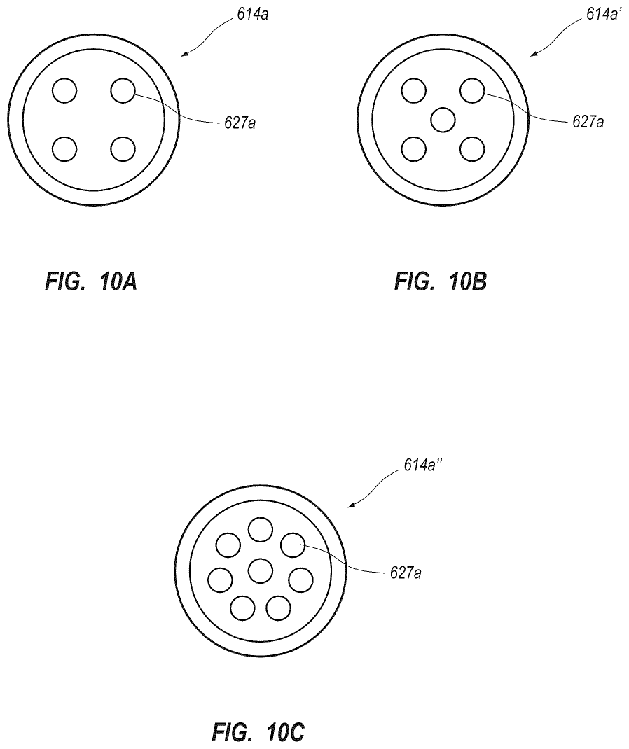

FIG. 10A illustrates an embodiment of a port 614a that may be present in certain varieties of control machines 601. The port 614a includes four electrical contacts 627a, such as electrical sockets, that may typically receive electrical contacts (e.g., pins) of the connector of a standard RF probe. The standard RF probe may include an electrode and a temperature sensor (e.g., a thermocouple), and at least some of the various electrical contacts 627a may be used to deliver electrical energy to, receive electrical energy from, and/or communicate electrical signals to and/or from the electrode and the temperature sensor of an RF probe that is connected to the port 614. For example, in some embodiments, only two of the electrical contacts 627a are active so as to communicate with the RF electrode and/or a thermocouple positioned therein.

FIG. 10B illustrates a further embodiment of a port 614′ that may be used in certain varieties of control machines 601. The port 614′ includes five electrical contacts 615. In some embodiments, two of the electrical contacts 627a may be used to deliver electrical energy to, receive electrical energy from, and/or communicate electrical signals to and/or from the electrode and the temperature sensor of an RF probe that is connected to the port 614′. A third of the electrical contacts 627a may be used for supplemental power. The supplemental power may be used by an adapter 670, as further discussed below. Other arrangements of ports 614 and/or other numbers of electrical contacts 627a are contemplated. For example, in some embodiments, two additional electrical contacts 627a may be used for providing supplemental power to an instrument assembly or other device, such as the adapter 670. As further discussed below, embodiments of adapters 670 may use the supplemental power available from such ports to power specialized circuitry, such as for purposes of energizing (e.g., selectively energizing) and/or otherwise controlling indicators on the adapters 670 and/or on instrument assemblies that are attached to the adapters 670. In some embodiments, the port 614′ may include still further dummy electrical contacts, such as previously described.

FIG. 10C illustrates yet a further embodiment of a port 614″ that may be used in certain varieties of control machines 601. The port 614″ includes eight electrical contacts 615. In some embodiments, two of the electrical contacts 627a may be used to deliver electrical energy to, receive electrical energy from, and/or communicate electrical signals to and/or from the electrode and the temperature sensor of an RF probe that is connected to the port 614″. A third of the electrical contacts 627a may be used for supplemental power.

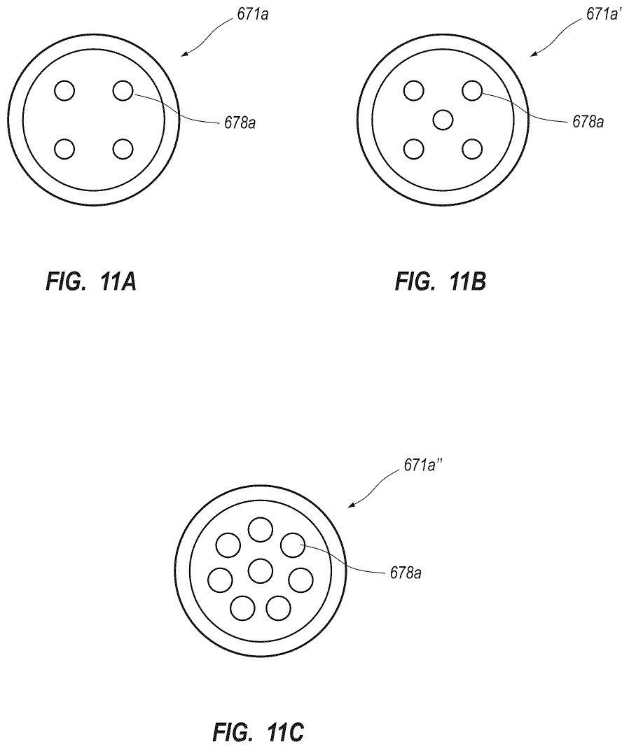

FIGS. 11A-11C depict embodiments of proximal connectors 671a, 671a′, 671a″ that are configured to physically attach to and electrically couple with the active electrical contacts of the ports 614, 614′, 614″ described above, respectively. In the illustrated embodiments, the numbers and configurations of electrical contacts 678a of the proximal connectors 671a, 671a′, 671a″ are complementary to those of the electrical contacts 627a of the ports 614a, 614a′, 614a″, respectively.

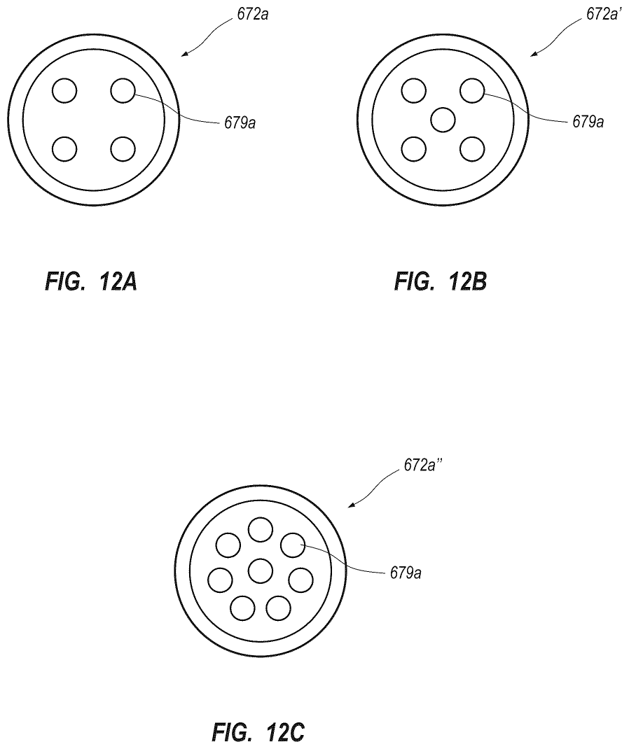

FIGS. 12A-12C depict embodiments of distal connectors 672a, 672a′, 672a″ that are configured to physically attach to and electrically couple with the proximal connectors of different varieties of instrument assemblies, such as the instrument assemblies 604a-d of FIG. 9. Other configurations for the distal connectors 672a, 672a′, 672a″ are contemplated. In some embodiments, the number and configuration of electrical contacts 679a are the same as or similar to those of the electrical contacts 678a of the proximal connectors 671a, 671a′, 671a″, respectively. However, in further embodiments, the distal connectors 672a, 672a′, 672a″ can have different numbers and/or arrangement of electrical contacts 679a. Indeed, in many embodiments, the proximal and distal connectors of the adapters will be arranged very differently from one another, as a purpose of the distal connector is to attach to the proximal connector of an instrument assembly that is not necessarily connectable to the port of the control machine. Stated otherwise, an arrangement of the proximal connector of an adapter and an arrangement of the proximal connector of an instrument assembly to which the adapter is configured to couple via the distal connector of that adapter may be significantly different. In some embodiments, the distal connectors 672a, 672a′, 672a″ comprise more electrical contacts 678a than do their proximal connector counterparts.

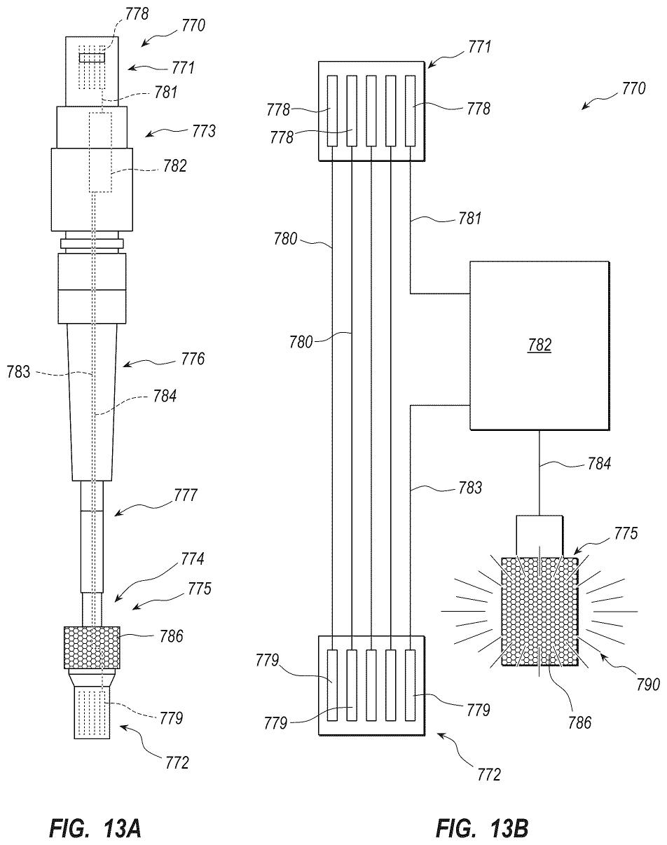

FIGS. 13A and 13B depict an embodiment of an adapter 770 that can be used in the system 600. For example, in some instances, the adapter 770 may be identical to any of the adapters 670a-d. In other instances, the adapter 770 may have some differences relative to one or more of the adapters 670a-d. In further instances, the adapter 770 may be used in place of one of the adapters 670a-d, and in still further instances, may be used alongside one or more of the remaining adapters 670a-d within the system 600.

FIG. 13A is an elevation view of the adapter 770, whereas FIG. 13B is a schematic diagram of certain components of the adapter 770. As shown in FIG. 13A, in some embodiments, the adapter 770 defines a relatively short length, as compared, e.g., with an instrument assembly that may be attached thereto. The adapter 770 includes a proximal connector 771 at a proximal end thereof and further includes a distal connector 772 at a distal end thereof. The proximal connector 771 is configured to directly connect to a port of any suitable control machine 601. The distal connector 772 is configured to directly connect to a proximal connector of an instrument assembly, such as, e.g., embodiments of the connectors 630a-d of the instrument assemblies 604a-d shown in FIG. 9.

In other or further embodiments, the distal connector 772 can connect directly to the connector 130a-d of any of the instrument assemblies 130a-d discussed previously. Further, the adapter 770 can include circuitry and/or controls that achieve any or all of the identification functionalities previously described. For example, as previously mentioned, the adapter 770 can include a controller (which may include one or more processors) and/or other control circuitry that can carry out any of the control and/or other operations that pertain to activating, deactivating, and/or otherwise controlling visual indicia displayed by the instrument assembly when such is coupled to the distal connector of the adapter 770. Accordingly, in some embodiments, the adapters 770 can act substantially as specialized port devices that convert ports of control machines that are devoid of instrument assembly identification functionalities to ports that provide identification functionality. Thus, for example, embodiments of the adapters 770 can include one or more features of the ports 114a-d and control thereof (e.g., via the processor 109) previously discussed, and may be capable of powering and/or controlling one or more of the instrument assemblies 104a-d, 204a-d, 304a-d, 404a-d in manners such as previously discussed.

In the illustrated embodiment, the adapter 770 includes a proximal hub 773 and a distal hub 774 to which the proximal and distal connectors 771, 772, respectively, are attached. The adapter 770 further includes a flexible strain-relief member 776 and a flexible cord or stem 777. In this embodiment, the flexible strain-relief member 776 and the flexible stem 777 permit the connectors 771, 772 to be movable or adjustable relative to one another, which may facilitate use of the adapter 770, in some instances. For example, in some instances, a user may attach the proximal connector 771 to a port of a control machine and then bend the stem 777 and/or the strain-relief member 776 to a more reachable or comfortable orientation before attaching an instrument assembly to the distal connector 772.

In other embodiments, the adapter 770 may be longer or shorter than what is depicted in FIG. 13A. For example, the adapter 770 may be significantly shorter. In some embodiments, the strain-relief member 776 and the stem 777 are omitted. In still further embodiments, the proximal and distal connectors 771, 772 may be adjoined to a single hub (e.g., one of the proximal or distal hubs 773, 774 may be omitted), and, thusly secured to a single hub, the connectors 771, 772 may be in a fixed relationship relative to each other. For example, when the proximal connector 771 is connected to a port, the distal connector 772 may project away from that port, such that the adapter 770 may appear as an extension of the port, to which the proximal connector of an instrument assembly may then be attached. The connectors 771, 772 may extend or project in opposite directions.

With reference to FIGS. 13A and 13B, the proximal connector 771 can include any suitable arrangement and number of electrical contacts 778. The electrical contacts 778 can be positioned and sized to interface or interconnect with the electrical contacts 615 of a port 614, 614′, 614″ (see FIGS. 10A-10C). For example, in some embodiments, the electrical contacts 778 are formed as pins that fit into sockets of a port 614, 614′, 614″. In the illustrated embodiment, the proximal connector 771 includes five electrical contacts 778. In certain embodiments, the electrical contacts 778 are pins that are sized and arranged to fit within five sockets of a port (e.g., the port 614′). The electrical contacts 778 are arranged in the same X-shaped arrangement of the sockets 615 depicted in FIG. 10B, such that two of the five electrical contacts 778 are hidden behind the two outer electrical contacts of the three electrical contacts 778 that are depicted in phantom in FIG. 13A. The full set of five electrical contacts 778 is depicted schematically in FIG. 13B.

In other embodiments, more or fewer electrical contacts 778 may be present. For example, in some embodiments, the proximal connector 771 includes four electrical contacts 778, and in some instances, the proximal connector 771 can be configured to connect with a port such as the port 614 of FIG. 10A. In other embodiments, the proximal connector 771 includes eight electrical contacts 778, and in some instances, the proximal connector 771 can be configured to connect with an eight-socket port, such as the port 614″ of FIG. 10C. In some instances, one or more sockets of a port may be used in the supply of supplemental power, as previously discussed.

The distal connector 772 can include any suitable arrangement and number of electrical contacts 779. The electrical contacts 779 can be positioned and sized to interface or interconnect with the electrical contacts of a proximal connector of an instrument assembly, such as any of the proximal connectors 630a-d of the instrument assemblies 604a-d (FIG. 9). For example, in some embodiments, the electrical contacts 779 are formed as sockets for receiving pins of the proximal connector of an instrument assembly (e.g. an instrument assembly 604a-d).

In FIG. 13A, only three of five total electrical contacts 779 are visible, as two may be hidden from view by other of the electrical contacts 779 that are depicted in phantom, in manners such as discussed above with respect to the proximal connector 771. The full set of five electrical contacts 779 is schematically depicted in FIG. 13B. Other numbers and arrangements of electrical contacts 779 are contemplated. For example, in various embodiments, the distal connector 772 includes four, five, six, seven, or eight electrical contacts 779.

With reference to FIG. 13B, four of the electrical contacts 778 of the proximal connector 771 are routed directly to four of the electrical contacts 779 of the distal connector 772. That is, for four of the electrical contacts 778, each electrical contact 778 is directly connected to one of the electrical contacts 779 via an electrical lead 780. In certain embodiments, these four sets of electrical contacts 778, 779 and electrical leads 780 effectively transmit control signals and/or ablation energy from the control machine to the instrument assembly and/or transmit electrical energy and/or heat detection signals from the instrument assembly to the control machine, such as in manners typically employed by known control machines and instrument assemblies. These portions of the adapter 770, in effect, serve as an extension of the port to which the adapter 770 is coupled. That is, the adapter 770 may substantially serve as a pass-through device with respect to standard controls for, e.g., an RF probe.

In other embodiments, only two of the electrical contacts 778 of the proximal connector 771 are routed directly to two of the electrical contacts 779 of the distal connector 772. For example, such an arrangement may be used for a control machine that only uses two electrical contacts 779 to communicate with an RF probe and an associated thermocouple.

With reference to FIGS. 13A and 13B, a further electrical contact 778 of the proximal connector 771 may be configured to couple with an electrical contact of a port that provides supplemental power. This electrical contact 778 can be connected to a power line 781 that is in turn connected to a controller 782. The controller 782 may be of any suitable variety. In various embodiments, the controller 782 includes and/or is embodied in one or more processors, microprocessors, controller, microcontrollers, and/or printed circuit boards.

As previously mentioned, in some embodiments, the controller 782 is configured to provide functionalities carried out by the controller 109 of the control machine 101. For example, the controller 782 may be configured to obtain stored data from an RFID tag, EEPROM, and/or other memory device of an instrument assembly to which the adapter 770 is attached. The controller 782 may be configured to determine whether the instrument assembly includes a controllable indicator. The controller 782 may be configured to control the controllable indicator of the instrument assembly.

The controller 782 can provide one or more signals to the adapter indicator 775 to provide a visual indicium 790 that emanates from the adapter 770. In particular, the controller 782 can be connected to the indicator 775 via one or more suitable communication lines 784, which may be used to deliver communication signals and/or power to the indicator 775. In the illustrated embodiment, the indicator 775 includes an LED unit 785, which can include an LED or an LED array. The indicator 775 can further include an optical element 786 of any suitable variety to disperse light from the LED unit 785. For example, the optical element 786 may include one or more of a diffuser and a light pipe. In some embodiments, the light pipe may be a cylindrical element that fully extends about a periphery of the adapter 770. In the illustrated embodiment, the indicator 775 is at or near the distal connector 772. As discussed below, in other embodiments, the indicator 775 may be at a more proximal position on the adapter 770. In the illustrated embodiment, the visual indicium 790 is colored light.

Any of arrangements discussed with respect to the indicators 145a-d is contemplated with respect to the indicator 775. In other or further embodiments, a large portion, or even an entirety, of the stem 777 can light up, such as by using a clear or translucent jacket and a plurality of LEDs embedded throughout the cable length of the stem 777. In other or further embodiments, an optical fiber is embedded in the stem 777 to light up a full or partial length thereof with one LED or with a single LED assembly. In other or further embodiments, the stem 777 lights up in a manner that simulates flow in the direction of the patient when energy is being distributed from that port. Such lighting arrangements may also be employed with embodiments of the instrument assemblies disclosed herein.

The controller 782 may further be connected to an electrical contact 779 by way of a further communication line 783. The communication line 783 may be configured to deliver one or more of communication signals and/or power to the electrical contact 779, which may in turn be coupled with an indicator 645a-d of an instrument assembly 604a-d (FIG. 9). In some embodiments, control signals from the controller 782 can be delivered to an indicator 645a-d directly to control the indicator 645a-d. For example, in some instances, a color of light produced by an LED unit may be controlled by a voltage level, current level, or in some other manner. In some embodiments, pulse width modulation is used to control a level of light produced by each LED within an LED array. In other embodiments, control signals from the controller 782 may be delivered to a controller that is coupled to the indicator 645a-d, (e.g., within the instrument assembly), and can thereby indirectly control (e.g., control by way of the intermediary controller) the indicator 645a-d.

The controller 782 of the adapter 770 can control the indicator 775 to produce an indicium 790 that is associated with (e.g., that matches) an indicium produced by the indicator 645a-d of the instrument assembly 604a-d that is attached to the adapter 770 in manners such as previously discussed with respect to indicators 125, 225, 325, 425. Stated otherwise, the indicator 775 of an adapter 770 can serve as a port-associated indicator 775 that identifies the port to which the adapter 770 is attached.

Methods of using certain embodiments of the adapter 770 will now be described. In some embodiments, the controller 782 of the adapter 770 is preprogrammed to produce only one color (i.e., an indicium 790 of a single color of light) by way of the indicator 775. Any suitable number of varieties of adapters 770 may be available, each variety producing a different color. For example, for a system 600 (FIG. 9) that includes a control machine 601 having four ports 614a-d, at least four varieties of adapters 770 may be available, each variety producing only a single color of light, when in use, that differs from the colors produced by the other adapters 770. In order to individually identify each instrument assembly 604a-d and the individual port 614a-d to which it is attached by way of an adapter 770, a user can use individual adapters 770 of four different varieties (e.g., of four different colors). Each adapter 770 can be coupled to a different one of the ports 614a-d. Each controller 782 of the adapters 770 can cause the respective indicator 645a-d of each instrument assembly 604a-d attached thereto to produce light of a color that matches the light produced by the indicator 775 of that adapter 770. A user thus may perceive visual indicia displayed by the various adapters 770 and by the various instrument assemblies 604a-d coupled thereto and can readily determine correspondences of the respective adapters 770 and assemblies 604a-d, such as may be due to matching sets of colors.

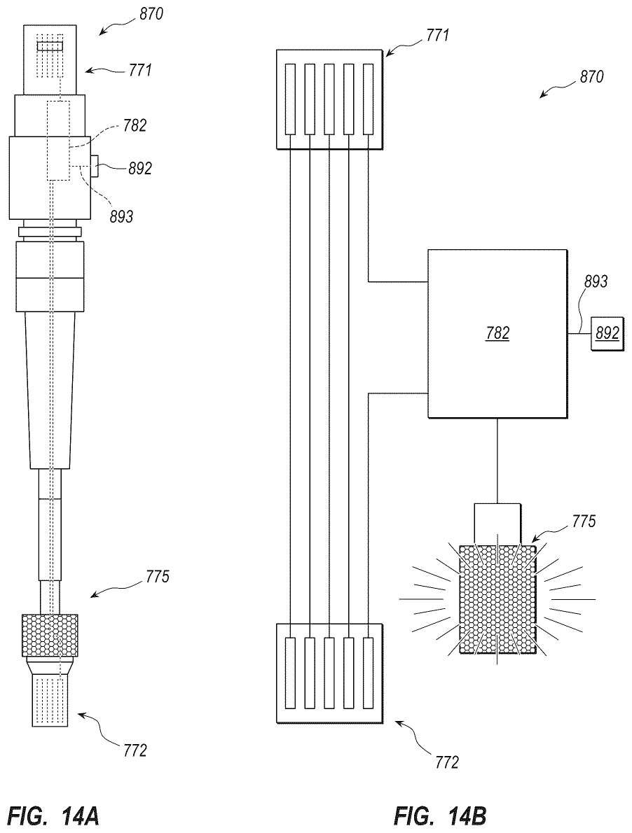

FIGS. 14A and 14B depict another embodiment of an adapter 870. The adapter 870 may be substantially identical to embodiments of the adapters 770 just described. In addition to the features of the embodiment of the adapter 770 depicted in FIGS. 13A and 13B, the embodiment of the adapter 870 depicted in FIGS. 14A and 14B includes an actuator 892. The actuator 892 communicates with the controller 782 through a communication line 893. The actuator 892 permits a user to select a color of light that is displayed by the indicator 785. In various embodiments, the actuator 892 can comprise a button, dial, knob, switch or other selection device of any suitable variety by which the user may provide control signals to the controller 782 to select a color of light.

In some embodiments, the controller 782 is preprogrammed with a predetermined number of color options, which a user can select via the actuator 892. For example, in various embodiments, the user can rotate a dial, slide a button to a predetermined position, or repeatedly press a button to cycle through the color options to arrive at a desired color. Other suitable actuation techniques for selecting one of the preset color options are also contemplated. The controller 782 can, as previously discussed, be preprogrammed to control the indicator of an instrument assembly that is coupled to the distal connector of the adapter 870 to display a matching color.

In other embodiments, the actuator 892 permits a user to select substantially any desired color. For example, the actuator may permit a user to assign a desired set of RGB values to an LED unit. The indicator 775 and the indicator of an instrument assembly coupled to the adapter 870 can display the same color selected via the actuator 892.

Methods of using certain embodiments of the adapter 870 will now be described. In some instances, four adapters 870 (which, in some instances, may be identical to each other) can be used in place of the adapters 670a-d depicted in FIG. 9. Each adapter 870 can be attached to a separate port 614a-d, and the instrument assemblies 604a-d can be attached to the distal connectors 772 of the respective adapters 870. The user can actuate the actuator 892 of one, two, three, or all four of the adapters 870 to select, for example, four different colors, with each adapter 870/instrument assembly 604a-d pair displaying a color that is distinct from the colors displayed by the remaining pairings. In some embodiments, for at least one of the adapters 870, the user selects one color from among a predetermined number of preset colors. In other or further embodiments, for at least one of the adapters 870, the user selects substantially any desired color, or stated otherwise, the adapter 870 does not include a small set of predetermined colors. A user thus may perceive visual indicia displayed by the various adapters 870 and by the various instrument assemblies 604a-d coupled thereto and can readily determine correspondences of the respective adapters 80 and assemblies 604a-d due to the matching sets of colors.

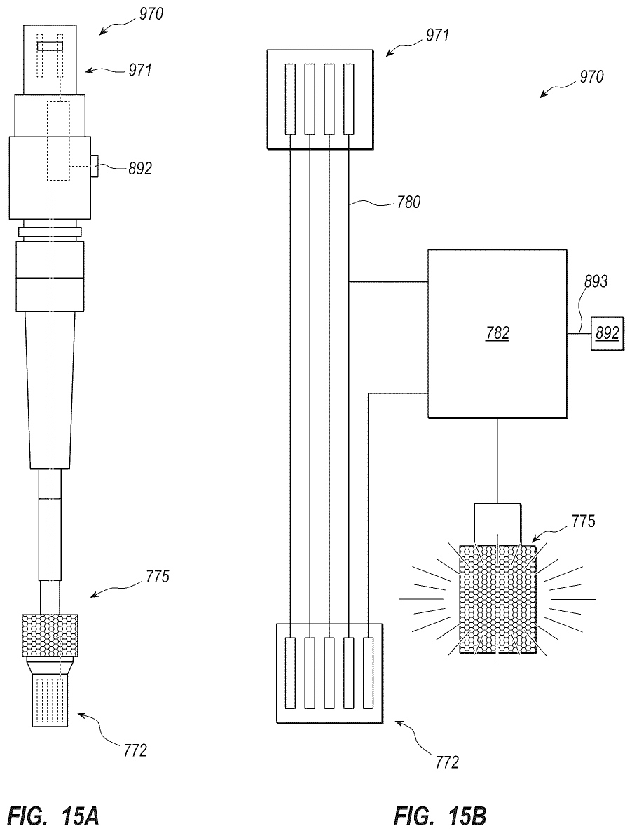

FIGS. 15A and 15B depict another embodiment of an adapter 970. The adapter 970 resembles embodiments of the adapters 770, 870 just described. For example, in some embodiments, the adapter 970 includes an actuator 892 via which a user can select a color of the indicator 775 in manners such as previously described. In other embodiments, the actuator 892 may be omitted, such as, for example, in certain instances where a single color is displayable via the indicator 775.

The adapter 970 can include a distal connector 772 such as previously described. In the illustrated embodiment, the adapter 970 includes a proximal connector 971 that differs from the proximal connector 771. In particular, the proximal connector 971 includes four electrical contacts 778, which may be configured to interface with a port such as the port 614 depicted in FIG. 10A.

In some embodiments, the adapter 970 is configured to draw power from one or more of the electrical lines 780 that extend directly between the proximal connector 971 and the distal connector 772, and which may provide controls and/or communication directly between a control machine and an instrument assembly. In the illustrated embodiment, one or more electrical lines 981 are coupled to one or more of the electrical lines 780 to provide power to the controller 782.

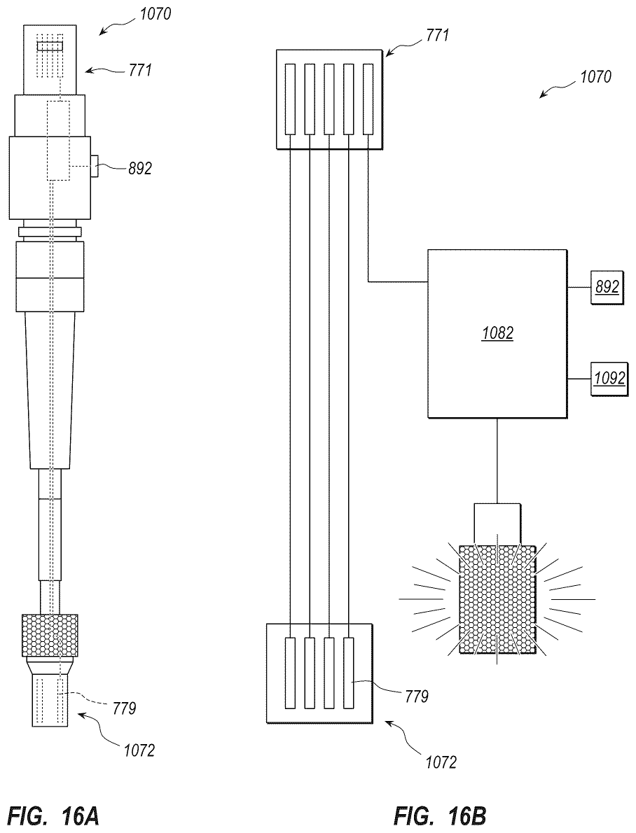

FIGS. 16A and 16B depict another embodiment of an adapter 1070. The adapter 1070 can resemble the adapter 870. For example, the adapter 1070 can include an actuator 892 coupled with a controller 1082 via which a user can select a color in manners such as previously described. The adapter 1070 can include a proximal connector 771 such as previously described, and can draw supplemental power from a control machine to power the controller 1082.

The adapter 1070, however, can have a differently configured distal connector 1072. In particular, in the illustrated embodiment, the distal connector 1072 includes four electrical contacts 779. The distal connector 1072 may permit typical control of an instrument assembly, such as an RF probe, via the four direct lines between the proximal connector 771 and the distal connector 1072. In other embodiments, fewer direct lines may be used in controlling an RF probe.

The controller 1082 does not communicate with an instrument assembly via the distal connector 1072, when the instrument assembly is attached to the distal connector 1072. Rather, the adapter 1070 includes a wireless communication member 1092 via which the controller 1082 can wirelessly communicate with an indicator of the instrument assembly. For example, in some embodiments, the wireless communication member 1092 includes an RFID reader. In some embodiments, the instrument assembly can have its own power source (e.g., a battery) or can draw power delivered from the control machine via one or more of the electrical contacts 779 of the distal connector 1072 to power a transceiver, a receiver, and/or an LED unit. In some embodiments, embodiments of the instrument assemblies 104a-d may be particularly well-suited for use with the adapter 1070.

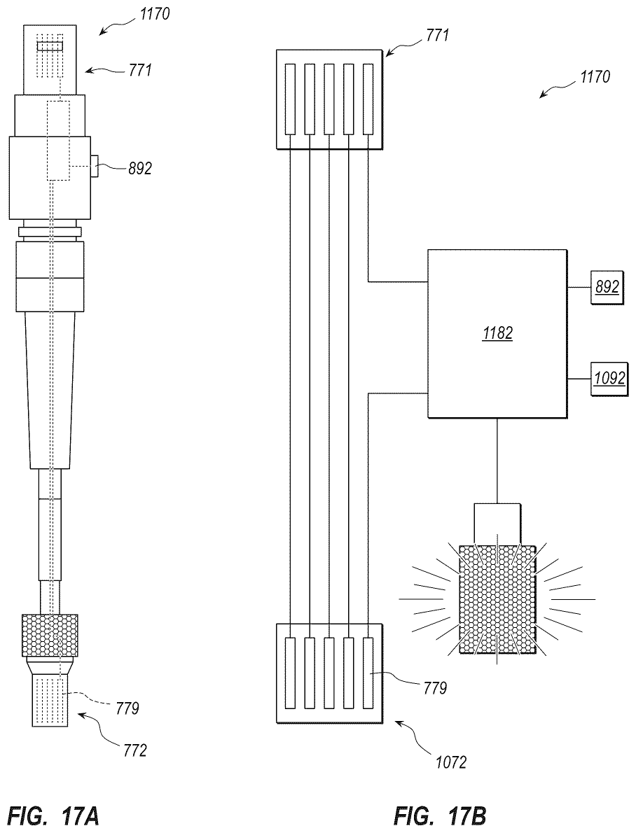

FIGS. 17A and 17B depict another embodiment of an adapter 1170. The adapter 1170 can resemble the adapter 1070. Unlike the illustrated adapter 1070, however, the adapter 1170 includes distal connectors 772 having an extra or supplemental electrical contact 779 by which a controller 1182 can provide power to a lighting circuit of an instrument assembly. The controller 1182 further includes a wireless communication member 1092 such as previously described. Accordingly, in some embodiments, the controller 1182 is configured to draw supplemental power from a control machine via one or more electrical contacts of the proximal connector 771 and is configured to provide power to the one or more extra electrical contacts of the distal connector 772. This supplemental power that is provided to an instrument assembly that is coupled to the distal connector 772 can be used to operate identification componentry of the instrument assembly, such as, e.g., by providing power to: a wireless receiver or transceiver that can wirelessly communicate with the wireless communication member 1092 of the adapter 1270; circuitry that is coupled with, e.g., an RFID tag that can receive signals from the wireless communication member 1092; and/or an LED assembly portion of an indicator for direct control thereof.

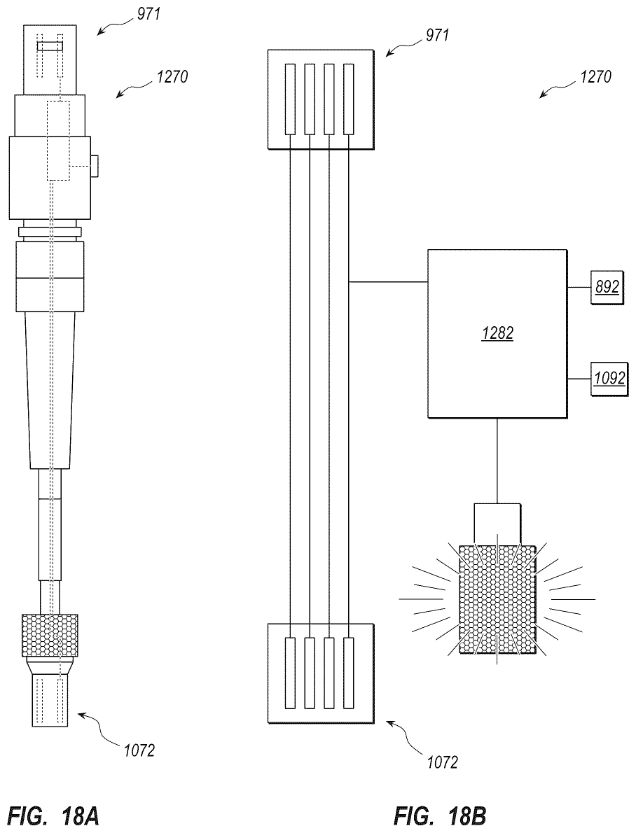

FIGS. 18A and 18B depict another embodiment of an adapter 1270. The adapter 1270 can resemble the adapters 1070, 1170. The adapter 1270 includes a proximal adapter 971 and a distal adapter 1072, as these components have previously been described. The adapter 1270 includes a controller 1282 that does not draw power from a control machine via any supplemental power supply, but instead draws power from one of the dedicated power lines that extend between the connectors 972, 1072. Moreover, the controller 1282 does not provide supplemental power and/or any communication through the distal connector 1072. The controller 1282 is configured to control an indicator of an attached instrument assembly by way of a wireless communication member 1092, such as previously described (e.g., via RFID communication and/or any other suitable wireless communication protocol).

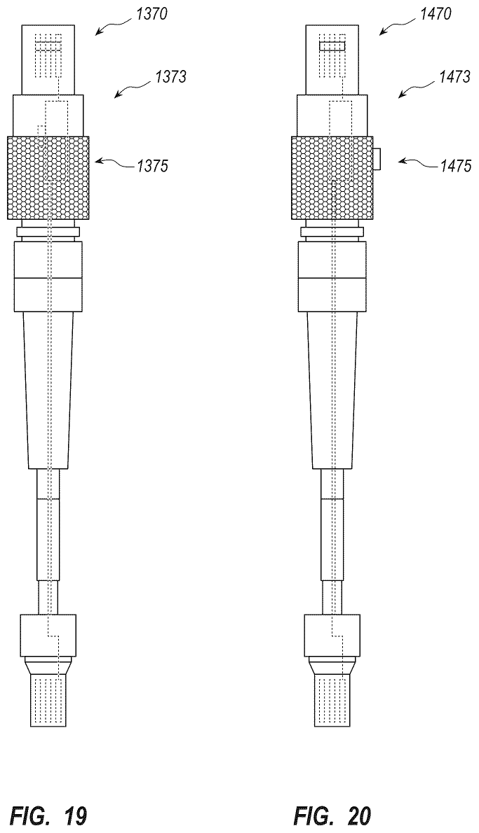

FIGS. 19 and 20 depict further embodiments of adapters 1370, 1470 that are similar to embodiments of the adapters 770, 870, respectively, which were previously described. The adapters 1370, 1470 include proximal hubs 1373, 1473. The primary difference between the adapters 1370, 1470 and the adapters 770, 870 is the positioning of the indicators. As previously discussed, the adapters 770, 870 include indicators 775, 875 at distal hubs. The adapters 1370, 1470 include indicators 1375, 1475 at their proximal hubs 1373, 1473. The indicators 1375, 1475 thus are more closely positioned to the ports of a control machine when coupled therewith. Any other and/or additional suitable placement(s) of the indicators 1375, 1475 is contemplated. Similar changes and/or additions to placements of other indicators of other adapters described herein are also contemplated.

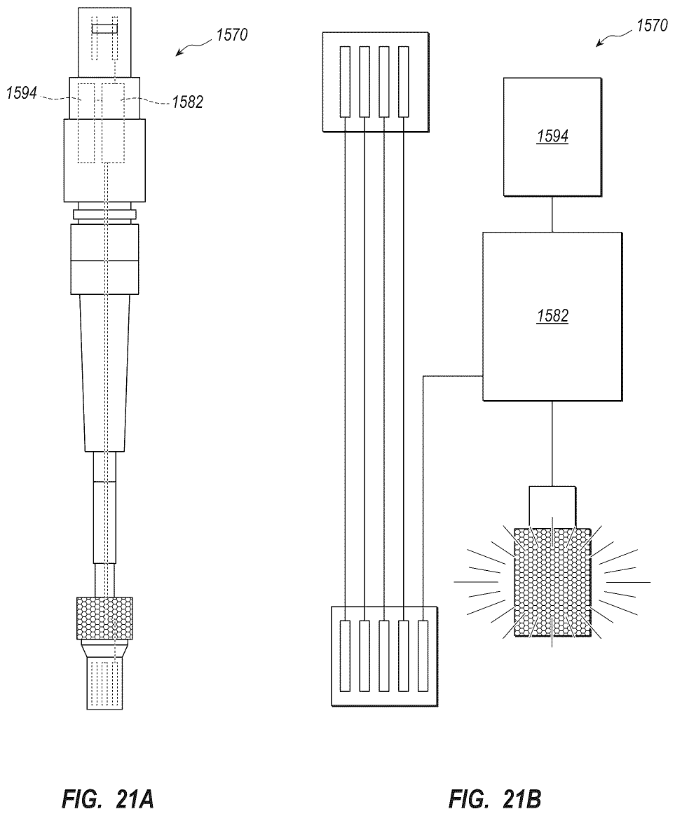

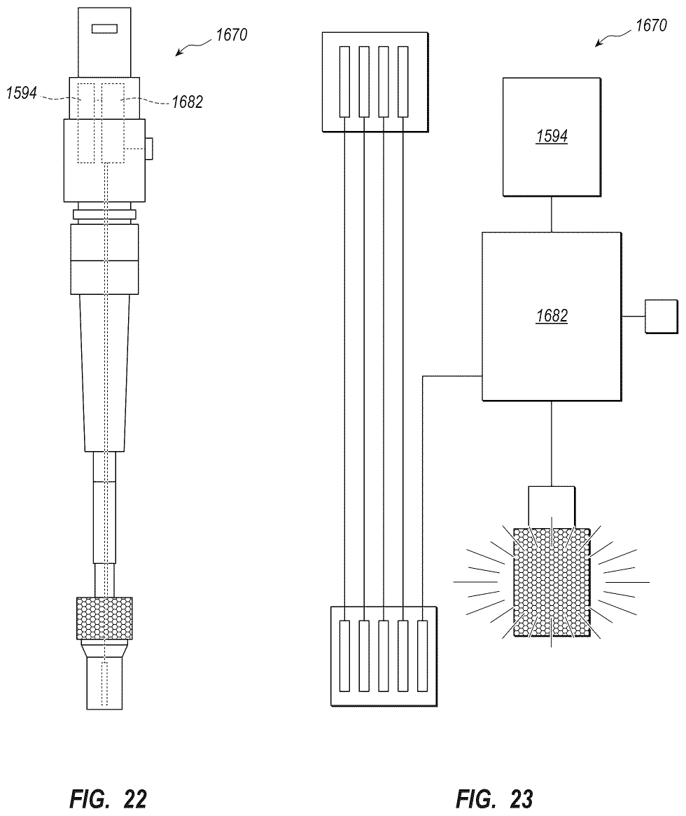

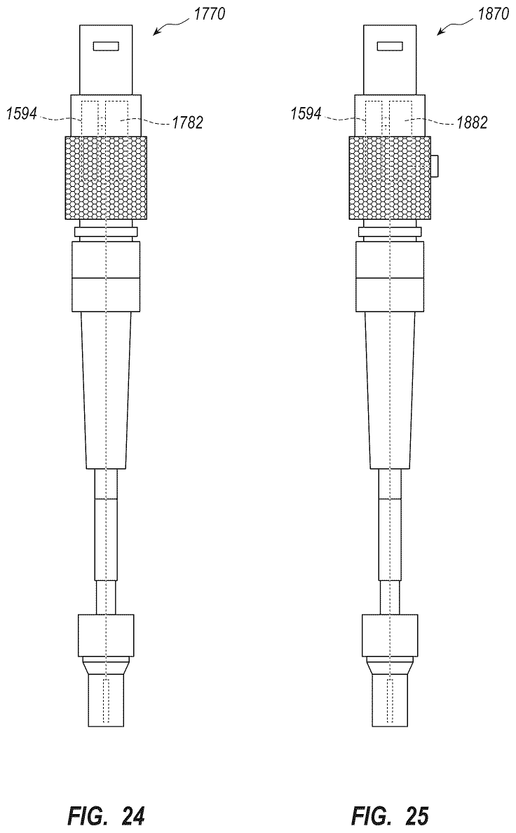

FIGS. 21A-25 depict embodiments of adapters 1570, 1670, 1770, 1870 that are similar to embodiments of the adapters 770, 870, 1370, 1470, respectively, which were previously described. The primary difference between the adapters 770, 870, 1370, 1470 and the adapters 1570, 1670, 1770, 1870 is that the former adapters include controllers that draw power from the control machine to which they are attached, whereas the latter adapters each include a power source 1594 that powers a respective controller 1582, 1682, 1782, 1882. In various embodiments, the power source 1594 can be described as independent, dedicated, onboard, local, and/or internal with respect to the adapter 770. The power source 1594 can be of any suitable variety. In various embodiments, the power source 1594 includes one or more batteries, which in some embodiments is rechargeable in any suitable manner. The one or more batteries can be removable in some instances. In other instances, the batteries are permanently enclosed within the adapter (e.g., and may be recharged via inductive charging).

In the illustrated adapters 1570, 1670, 1770, 1870, the power source 1594 powers the controller 1682, and the controller 1682 in turn supplies power to identification componentry of an instrument assembly when such is attached to a distal connector of the adapter. In other embodiments, the power source 1594 may supply power separately to each of the controller 1582, 1682, 1782, 1882 and to the instrument assembly when such is attached to the distal connector.

Any of the other adapters herein disclosed that draw power directly from a control machine can, in other embodiments, instead include a dedicated power source in manners such as just described.

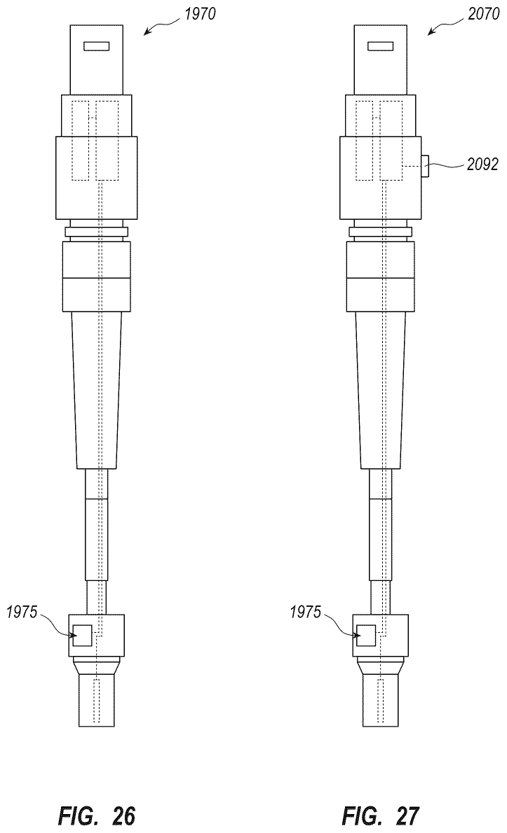

FIGS. 26 and 27 depict embodiments of adapters 1970, 2070 that are similar to embodiments of the adapters 1570, 1670, respectively, which were previously described. The primary difference between the adapters 1570, 1670 and the adapters 1970, 2070 is that the former adapters include indicators that, in some embodiments, only display colored light. In contrast, each of the adapters 1970, 2070 includes an indicator 1975 that can display colored light and/or any of a variety of other indicia. For example, in some embodiments, the indicator 1975 includes a small screen that can display one or more colors, characters, symbols, etc. to uniquely identify the adapter. An instrument assembly coupled with either of the adapters 1970, 2070 can display a matching indicium. The adapter 2070 further includes an actuator 2092 or input device by which a user can select, adjust, alter, assign, or otherwise control what is displayed on the indicator 1975.

Any of the other adapters herein disclosed can alternatively or additionally include an indicator 1975 such as just described, which can be located at any suitable position on the adapter.

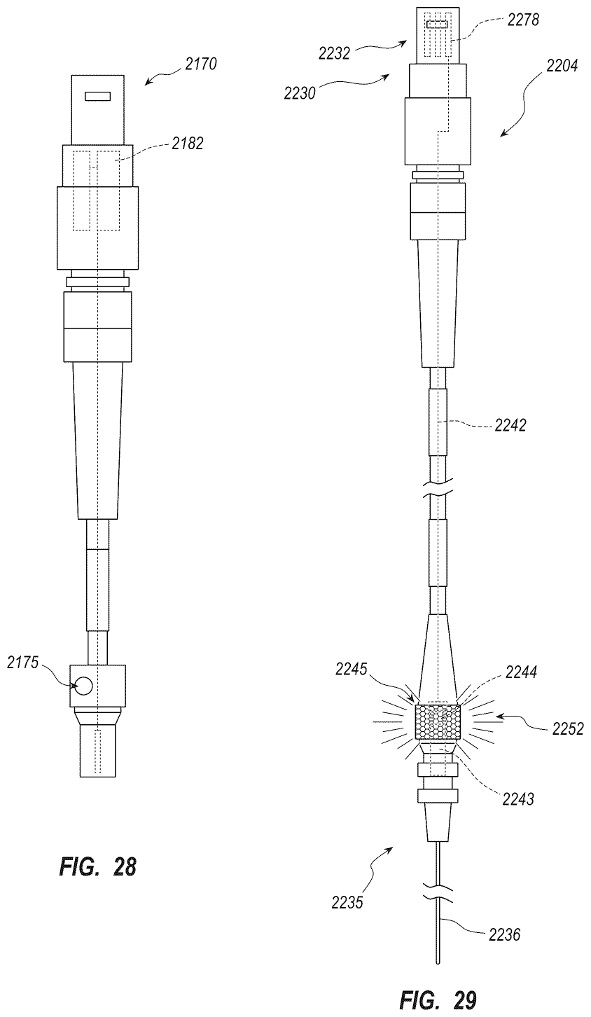

FIG. 28 depicts an embodiment of an adapter 2170 that is similar to embodiments of the adapters 1570, 1970, which were previously described. The primary difference between the adapters 1570, 1970 and the adapter 2170 is that the former adapters include indicators that utilize power in the delivery of their indicia, whereas the adapter 2170 includes a permanent indicator 2175 that does not require power for a display. For example, in various embodiments, the indicator 2175 may be a colored, stamped, printed, etched, or other element. The adapter 2170 can include a controller 2182 that controls the indicator of an instrument assembly that is attached to the adapter 2170 to achieve an association (e.g., a match with) the permanent indicator 2175.

Any of the other adapters herein disclosed can alternatively or additionally include an indicator 2175 such as just described, which can be located at any suitable position on the adapter.

FIG. 29 depicts an embodiment of an instrument assembly 2204 that is compatible with embodiments of adapters described herein. The instrument assembly 2204 can be similar to embodiments of the instrument assemblies 104a-g, which were previously described. The instrument assembly 2204 includes a proximal connector 2230 having a connection interface 2232. In the illustrated embodiment, the connection interface 2232 includes a communication interface, such as one or more electrical contacts 2278, that specifically permits communication with the controller of an adapter. The electrical contact or contacts 2278 is or are communicatively coupled with a controller 2243 of any suitable variety via a communication line 2242 of any suitable variety.

The controller 2243 can be include and/or be communicatively coupled with an LED unit 2244 or the like. The indicator 2245 can display a colored light indicium 2252, such as previously discussed. In some embodiments, the controller 2243 receives power and/or control signals form the controller of an adapter. The controller 2243 in turn controls the illumination (e.g., on/off status) and/or color provided by the LED unit 2244. In other embodiments, the controller 2243 is omitted, such that the controller of the adapter directly controls operation of the LED unit 2243 via one or more communication lines 2242. For example, in some embodiments, the controller of the adapter is configured to control individual colors of an RGB LED array by providing pulse-width modulation along three separate electrical channels.

As with other embodiments previously discussed, the instrument assembly 2204 can include an RF probe 2235, which can include an RF electrode 2236. Control signals for operating the RF electrode may be provided along further communication lines 2242 that extend through the instrument assembly 2204.

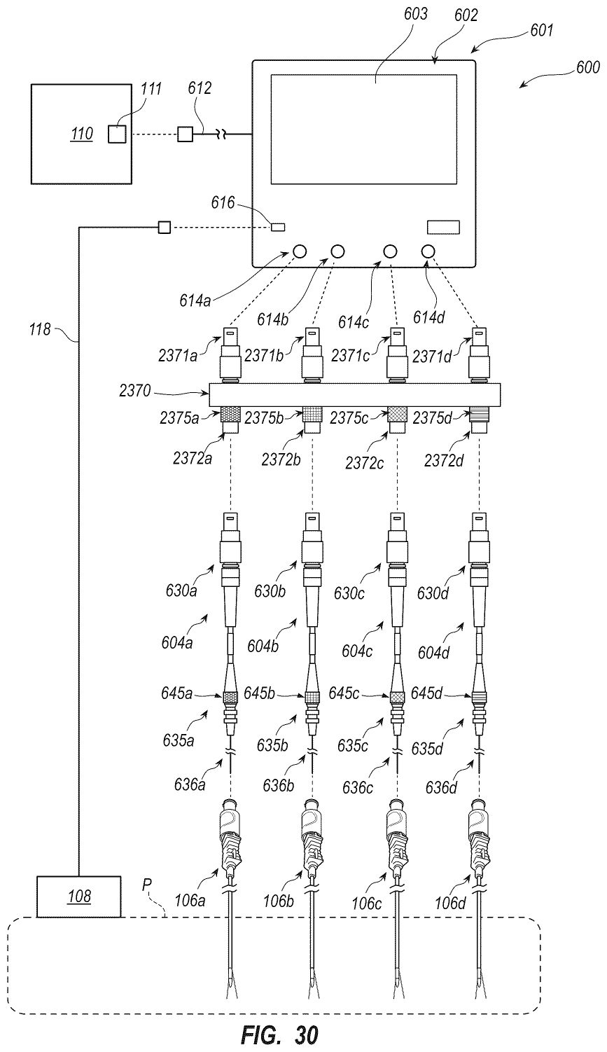

FIG. 30 depicts another embodiment of a system 2300 that can include the control machine 601 and the plurality of instrument assemblies 604a-d that are operable or controllable by the control machine 601. The system 2300 can resemble the systems 100, 600 previously described.

Rather than utilizing a plurality of independent adapters 670a-d, the system 2300 includes an adapter 2370 that is configured to simultaneously couple with each of the ports 614a-d of the control machine 601. The adapter 2370 includes a hub or housing 2373. Extending from a first side of the housing 2373 are a plurality of connectors 2371a-d, each of which is spaced and oriented so as to simultaneously couple with the ports 614a-d when the adapter 2370 is connected to the control machine 601. Each of the connectors 2371a-d is associated with a respective one of a plurality of ports 2372a-d that extend from a second side of the housing 2373. The ports 2372a-d may also or alternatively be referred to as distal connectors 2372a-d. In the illustrated embodiment, the connectors 2371a-d and the ports 2372a-d are positioned at opposite sides of the housing 2373 and extend in opposite directions. In the illustrated embodiment, each port 2372a-d is aligned (e.g., vertically aligned, when the adapter 2370 is vertically oriented as shown in FIG. 30) with the respective one of the connectors 2371a-d with which it is associated. The connectors 2371a-d can function in manners such as described above with respect to, e.g., the proximal connectors 671a-d, 771, 971, and the ports 2372a-d can function in manners such as described above with respect to, e.g., the distal connectors 672a-d, 772, 1072.

In the illustrated embodiment, each port 2372a-d includes an indicator 2375a-d at a base thereof. For example, in some embodiments, each of the indicators 2375a-d can be a light pipe or other lighting element, which may surround some or all of the base of the connector 2372a-d. The indicators 2375a-d can resemble in form and function any of the indicators previously described.

As described with previous embodiments, the indicators 2375a-d can be configured to display four unique visual indicia to assist a user in distinguishing one port from another, and these visual indica can correspond with unique visual indicia produced by the respective indicators 645a-645d of the instrument assemblies 604a-604d that are coupled to the individual ports 2372a-d in manners such as previously described.

In some embodiments, the adapter 2370 is configured to automatically ensure that each port 2372a-d displays a different indicium (e.g., a different color) relative to all of the remaining ports 2372a-d. For example, in some embodiments, each port 2372a-d is preprogrammed, preset, or otherwise configured to only display a single color of light via its respective indicator 2375a-d, with each color of light being different from that which is displayed by the remaining indicators (2375a-d). For example, the lights may be red, green, yellow, blue or some other combination of four distinct colors. In some embodiments, the adapter 2370 is devoid of actuators by which a user may select the color of a given port 2372a-d.

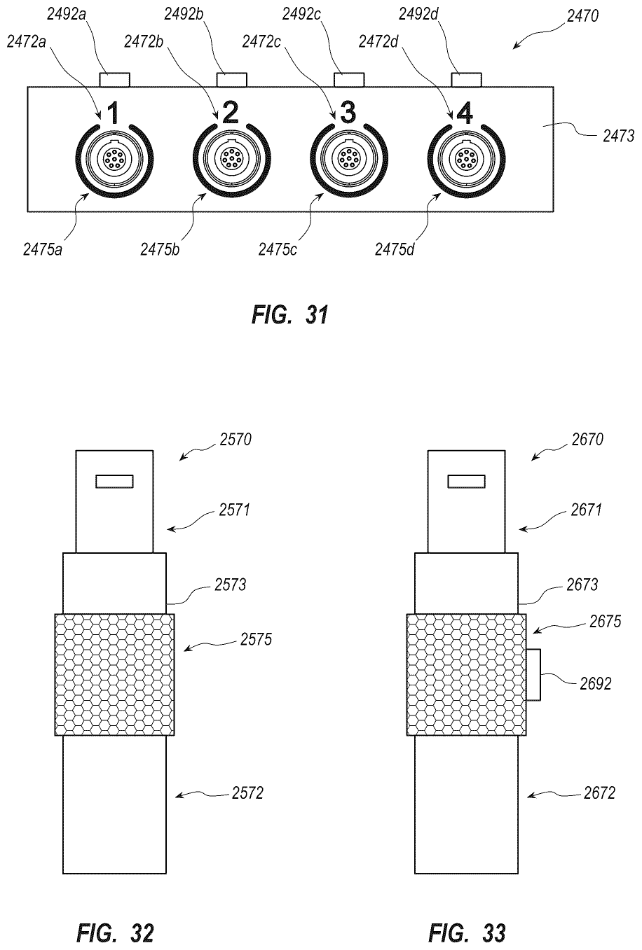

FIG. 31 depicts a front view of another embodiment of an adapter 2470, which can resemble the adapter 2370 in many respects. The adapter 2370 can include a housing 2473 much like the housing 2373. A plurality of connectors may extend from the housing 2473. The connectors are not shown in the view of FIG. 31, as they are on a side of the housing 2473 that is opposite from the side that is shown in FIG. 31. The connectors can resemble in form and function the connectors 2371a-d depicted in and described with respect to FIG. 30.

The adapter 2470 can further include a plurality of distal connectors or ports 2472a-d. The ports 2472a-d can resemble other ports or distal connectors previously described. In the illustrated embodiment, each port 2472a-d includes seven electrical contacts. In other embodiments, fewer electrical contacts may be present. For example, in various embodiments, the ports 2472a-d may include four electrical contacts or five electrical contacts. The ports 2472a-d can be of any suitable configuration to couple with various embodiments of proximal connectors (e.g., 2230) of instrument assemblies (e.g., 2204).

In the illustrated embodiment, the adapter 2470 includes a plurality of actuators 2492a-d via which a user can select a color displayed by a corresponding one of a plurality of indicators 2475a-d. The actuators 2492a-d may include any suitable mechanism, such as a button. In some embodiments, a user may toggle or advance through a preset selection of colors. The advancement may increment through the selections in a repeating manner or in a predetermined sequence (e.g., red, green, blue, orange, red, green, blue, orange, etc.).

In some embodiments, when one of the actuators 2492a-d is actuated to select a desired indicium (e.g., a particular color of light), the actuation can have no effect on the colored light or other indicia displayed by the indicators 2475a-d associated with the remaining actuators 2492a-d. In other embodiments, the adapter 2470 can include suitable circuitry, logic, microcontrollers, hardware, software, and/or other componentry or mechanisms to ensure that when one of the actuators 2492a-d is actuated is actuated to select a desired indicium (e.g., a particular color of light), the colored light or other indicia displayed by one or more of the indicators 2475a-d associated with the remaining actuators 2492a-d changes to ensure that each of the four indicators 2475a-d displays a visual indicium that is different or distinct from the indicia displayed by the remaining indicators 2475a-d.

Certain embodiments of other adapters discussed herein, such as embodiments of the adapters 670a-d, 770, 870, 970, 1070, 1170, 1270, 1370, 1470, 1570, 1670, 1770, 1870, 1970, 2070, 2170, 4570, 4670, can similarly be configured to operate within a system such that each adapter displays a different visual indicium from each of the remaining adapters that operate within that system. For example, in some embodiments, each adapter includes a single proximal connector and a single distal connector and is thus able to form a one-to-one correspondence between a port of a control machine and an instrument assembly, and multiple of such adapters are used within a system—one per port of the control machine—as previously described. In some instances, a first adapter may configured to select a color for display, e.g., randomly, in a predetermined manner, or otherwise. The remaining adapters (e.g., second, third, and fourth adapters) may communicate with the first adapter (e.g., via Bluetooth or other wireless protocol) to determine which color is being displayed thereby, and may similarly communicate with each other, to ensure that each adapter displays a color that is different from the others. In some embodiments, one or more of the adapters each can include an actuator (e.g., such as an actuator 892) by which a user can select which color is displayed on one or more of the adapters, and when this color or colors is/are selected, the one or more of the remaining actuators may select and/or change colors automatically to ensure that each adapter displays a different color.

FIG. 31 depicts another embodiment of an adapter 2470. The adapter 2470 can resemble the adapter 2370. For example, the adapter 2470 can include a plurality of connectors, each of which is spaced and oriented so as to simultaneously couple with the ports (e.g., the ports 614a-d in FIG. 30) of a control machine. These connectors can resemble those depicted in FIG. 30, but are not visible in the view of the illustrated embodiment, as the connectors project away from the viewer at a side of a housing 2473 that is opposite from the viewer.

The adapter 2470 further includes a plurality of ports 2472a-d, which can function in manners such as previously described with respect to the ports 2372a-d. The adapter 2470 further includes a plurality of indicators 2475a-d and a plurality of actuators 2492a-d, a respective one of each of which is associated with one of the ports 2472a-d. The actuators 2492a-d can be actuated by a user to select a desired color to be displayed by the respective indicator 2475a-d of a respective port 2472a-d.

In some embodiments, when a color is manually selected via one of the actuators 2492a-d for display by one of the indicators 2475a-d, the colors displayed by the remaining indicators 2475a-d can automatically adjust (e.g., remain the same or change) to ensure that each indicator 2475a-d displays a different color.

FIG. 32 depicts another embodiment of an adapter 2570. The adapter 2570 can resemble many of the adapters described elsewhere herein, such as, e.g., any of the adapters 670, 770, 1370, 1570, 1770, 1970, 2170, 4570, 4670. The adapter 2570, however, can be relatively shorter and have a single housing or hub 2573, in manners such as previously described. The adapter 2570 can include a proximal connector 2571, a distal connector or port 2572, and an indicator 2575, which components can resemble similarly named and numbered components previously described.

FIG. 33 depicts another embodiment of an adapter 2670. The adapter 2670 can resemble many of the adapters described elsewhere herein, such as, e.g., any of the adapters 870, 970, 1070, 1170, 1270, 1470, 1670, 1870, 2070, 4570, 4670. The adapter 2670, however, can be relatively shorter and have a single housing or hub 2673, in manners such as previously described. The adapter 2670 can include a proximal connector 2671, a distal connector or port 2672, an indicator 2675, and an actuator 2692, which components can resemble similarly named and numbered components previously described.

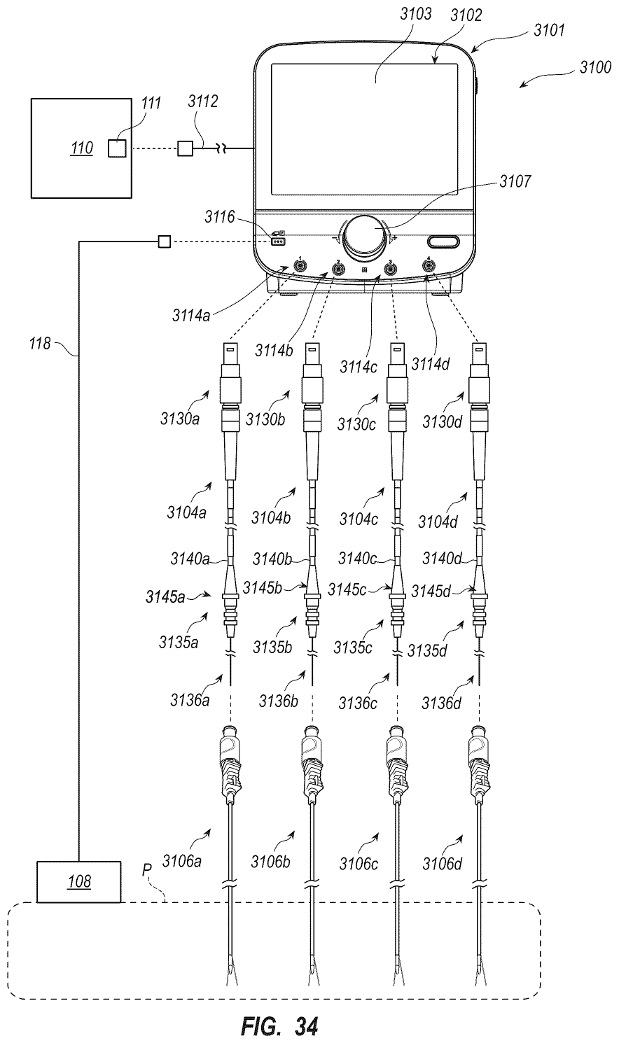

FIG. 34 depicts another embodiment of a system 3100 that resembles the system 100 previously described in certain respects. For example, the system 3100 includes the electrode pad 108, the electrical cable 118, the power source 110, the electrical outlet 111, and the such as previously named and described. Moreover, the system 3100 includes a control machine 3101, a display 102, a touchscreen 103, a control knob 107, and a grounding pad port 3116, which resemble embodiments of like named and like numbered (relative to the last two digits) components previously described. The system 3100 further includes numerous components and features that are numbered in manners similar to like components that have been previously described. In particular, the leading digits of such items have been incremented to “31.” Accordingly, it is understood that such prior disclosures regarding such features are equally applicable to the present embodiment.

The control machine 3101 can include a plurality of ports 3114-d, a plurality of instrument assemblies 3145a-d, and a plurality of medical instruments 3106a-d. As with embodiments of certain instrument assemblies previously described, each instrument assembly 3104a-d includes a respective proximal connector 3130a-d that is configured to couple interchangeably with any of the ports 3114a-d, a cable 3140a that includes an appropriate number and arrangement of communication lines therein, and a medical instrument at a distal end thereof, the medical instrument comprising, in particular, an RF probe 3135a-d that includes an electrode 3136a-d.

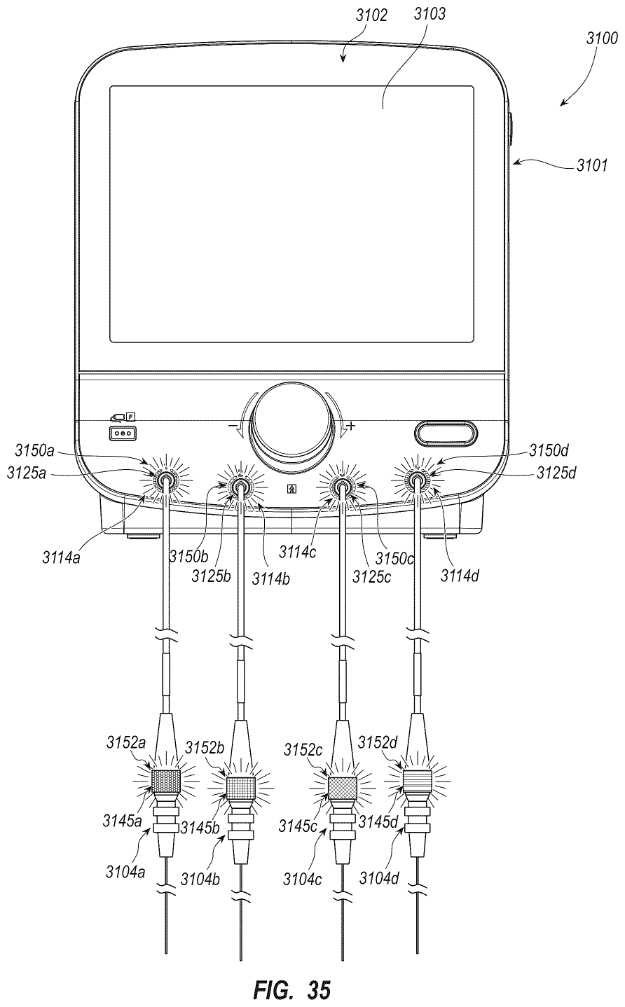

With reference to FIGS. 34 and 35, each instrument assembly 3104a-d includes an instrument or assembly indicator 3145a-d configured to display a visually perceptible indicium 3152a-d. Likewise, each port 3114a-d includes a port indicator 3125a-d configured to display a visually perceptible indicium 3150a-d. The visually perceptible indicia can be colored light.

In FIG. 36, various of the components previously discussed are shown schematically. FIG. 36 includes arrows among various of the components to demonstrate directions of communication, in the broader sense of the present application. For example, the power source 110 is in communication with a controller 3109, as the power source 110 is electrically coupled therewith to provide power thereto. The controller 3109 is further coupled with the touchscreen 3103, the control knob 3107, each of the ports 3114a-d, and the grounding pad port 3116. As shown by the arrows, the controller 3109 controls operation of the indicator 3125a-d of each of the ports 3114a-d. This control can include, for example, delivery of control signals to an LED array, as previously discussed. With respect to a connector 3120a-d of each port 3114a-d, the controller 3109 includes unidirectional communication toward the connectors 3120a-d and away from the connectors 3120a-d, and further includes two-way communication with the connectors 3120a-d. Similar flow is seen between the connectors 3120a-d, 3130a-d.

In some embodiments, the controller 3109 is configured to provide power to the connectors 3120a-d, such as for powering the indicators 3145a-d, and/or provide RF control signals for controlling the RF electrodes 3136a-d. In the illustrated embodiment, each of the instrument assemblies 3104a-d can include a memory device 3151a-d, which can include stored data regarding the presence of each indicator 3145a-d. Each instrument assembly 3104a-d further includes a thermocouple 3199b, which can be positioned within the RF electrode 3136b.

The controller 3109 can include architecture such as previously described with respect to the controller 109. Likewise, in various embodiments, the controller 3109 can function in any manner such as previously described with respect to the controller 109. Moreover, systems and methods previously described and/or described below, such as within the enumerated examples section, are fully associable with the system 3100, the controller 3109, and with other components of the system 3100, as they are with other systems, controllers, and other components described elsewhere herein.

With reference to FIG. 37, each port 3114a can be arranged substantially as previously discussed with respect to the port 114e in FIG. 3A. This drawing shows illustrative communication lines that can extend between the controller 3109 and components of the port 3114a. As further discussed below, the port 3114a can include eight electrical contacts 3127a via which the controller 3109 can control operations of the instrument assembly 3104a coupled therewith. FIG. 37 is shown schematically and depicts a connector 3120a that includes only five electrical contacts 3127a.

As further discussed below, and with additional reference to FIG. 38, embodiments of the port 3114a can include eight such electrical contacts 3127a via which the controller 3109 can control various functions of the instrument assembly 3104a and/or receive information from the instrument assembly 3104a.

With reference to FIGS. 37 and 38, the illustrated port 3114a includes a light guide 3126, which may be flush with or recessed relative to a front face of the control machine 3101. In the illustrated embodiment, a front covering of the control machine 3101 covers a portion of the light pipe such that the indicator 3125a includes an arcuate shape that encompasses a majority of the connector 3120a and a numeral (i.e., “1”), each of which is illuminated when an LED unit 3124a is active.

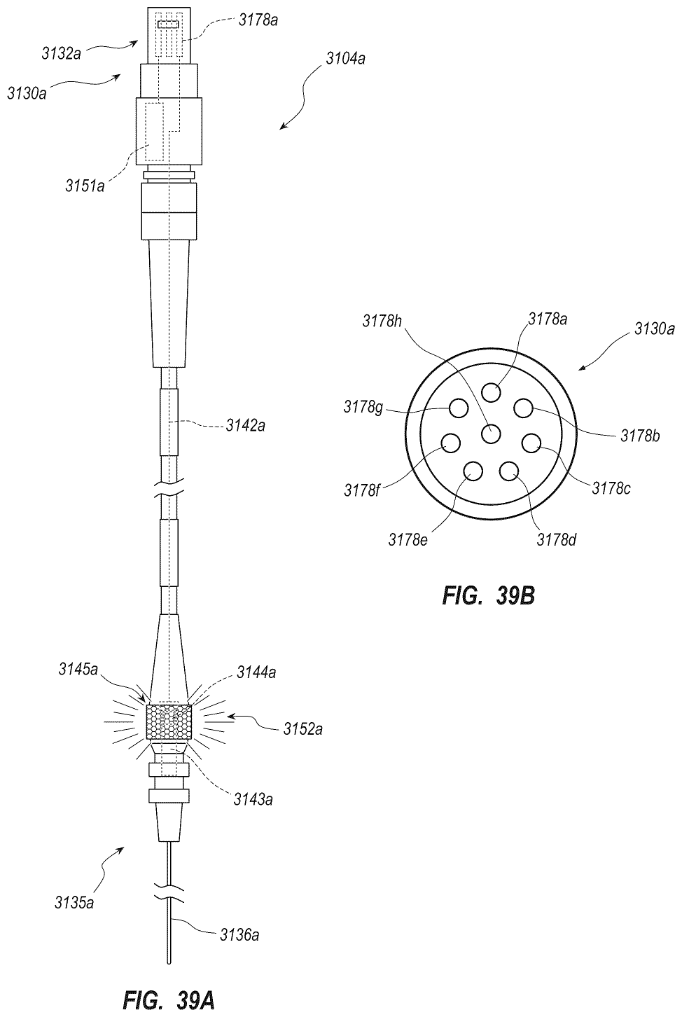

With reference to FIGS. 39A and 39B, the instrument assemblies 3104a-d, of which the instrument assembly 3104a is representative, can each include a connector 3130a with a connection interface 3132a that is complementary to a connection interface of the connector 3120a. In the illustrated embodiment, the connector 3130a includes eight electrical contacts 3178a-h. Each electrical contact 3178a-h can be coupled with a communication line 3142a.

With reference to FIG. 39A, in the illustrated embodiment, the instrument assembly 3104a includes a memory device 3151a, such as the memory device 151a discussed above. In the illustrated embodiment, the indicator 3145a includes a light source, such as an LED unit 3144a positioned within a hub of the instrument assembly 3104a. In some embodiments, the LED unit 3144a is coupled to a printed circuit board (PCB) 3143a, which may likewise be positioned within the hub. As previously noted, the instrument assembly 3104a includes an RF electrode 3136a and a thermocouple 3199a, which can be positioned within the RF electrode.

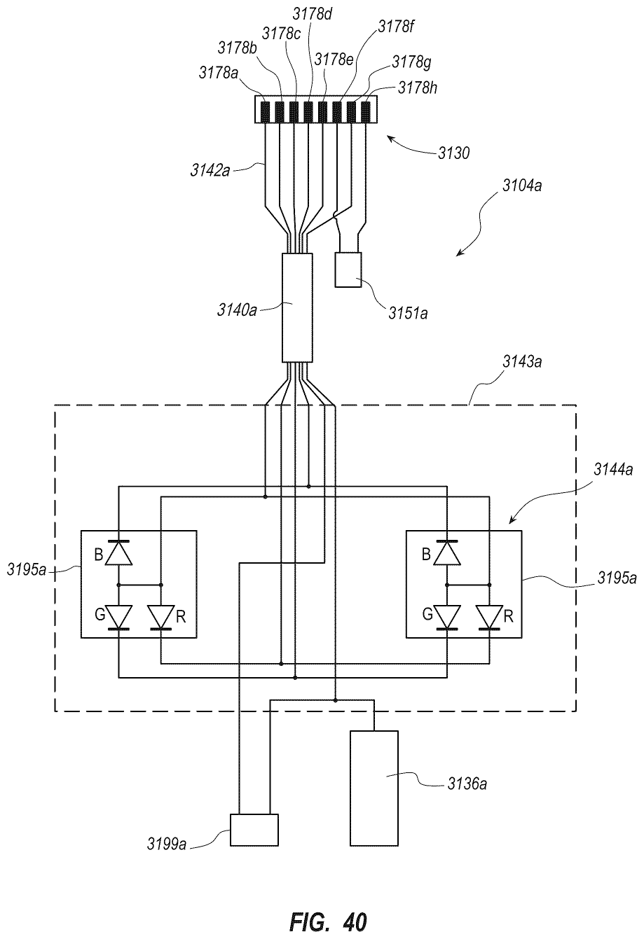

FIG. 40 provides a schematic diagram of components of the instrument assembly 3104a. The diagram depicts multiple communication lines 3142a that are in communication with the electrical contacts 3178a-h of the connector 3130. As depicted schematically, certain of the communication lines 3142a pass through a length of a cable 3140a. Proximal to the cable 3140a is the memory device 3151a, which in the illustrated embodiment is a single-wire EEPROM. Distal to the cable 3140a is the circuit board 3143a, and more distal still are the RF electrode 3136a and the thermocouple 3199a. The LED unit 3144a resides on the circuit board 3143a, and in the illustrated embodiment, the LED unit 3144a includes two LED arrays 3195a.

The electrical contacts 3178a-h are configured to couple with the port 3114a (or any of the ports 3114b-d) to establish key communications with the controller 3109. In the illustrated embodiment, the electrical contacts 3178a-h are configured to couple to the following:
3178a: an isolated direct current power supply at +5V;
3178b: red leg of the LED unit 3144a, 2V pulse width modulation provided by the controller 3109;
3178c: green leg of the LED unit 3144a, 3.3V pulse width modulation provided by the controller 3109;
3178d: blue leg of the LED unit 3144a, 3.3V pulse width modulation provided by the controller 3109;
3178e: negative branch of T-type thermocouple 3199a (constantan)
3178f: an isolated ground
3178g: a radiofrequency signal (0-150V) provided by the controller 3109; and
3178h: communication line of the single-wire EEPROM.

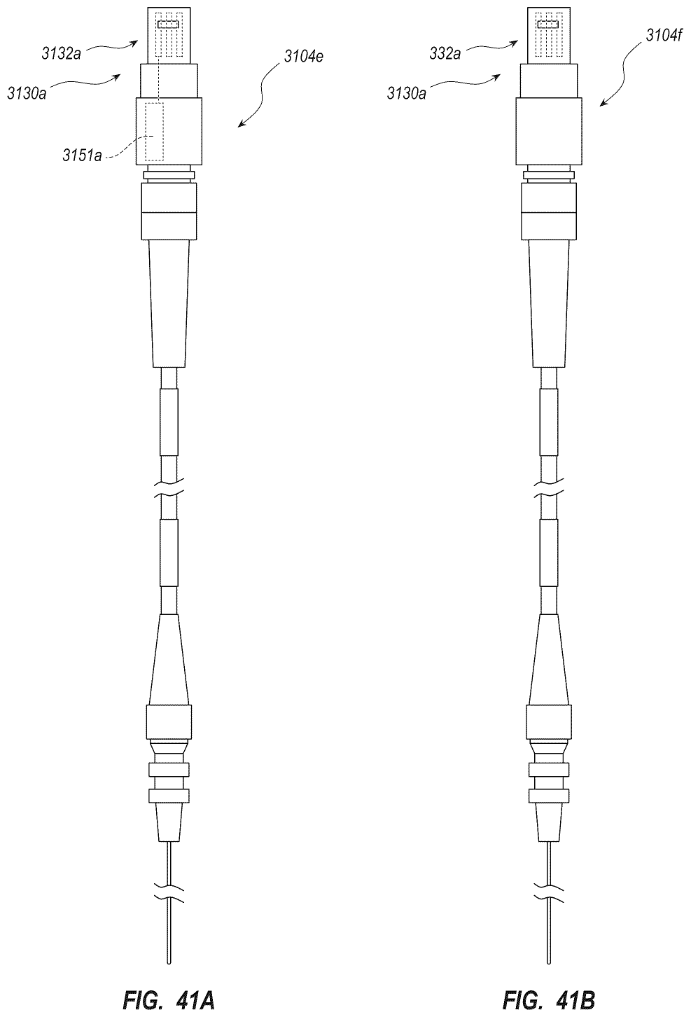

With reference to FIGS. 41A and 41B, the system can further include, and be configured to operate, with instrument assemblies 3104e, 3104f that are devoid of indicators, much as previously described with respect to the instrument assemblies 104f, 104g. The instrument assembly 3104e includes a memory device 3151a, which may communicate information regarding the lack of an indicator to the controller 3109. The instrument assembly 3104f is devoid of such a memory device.

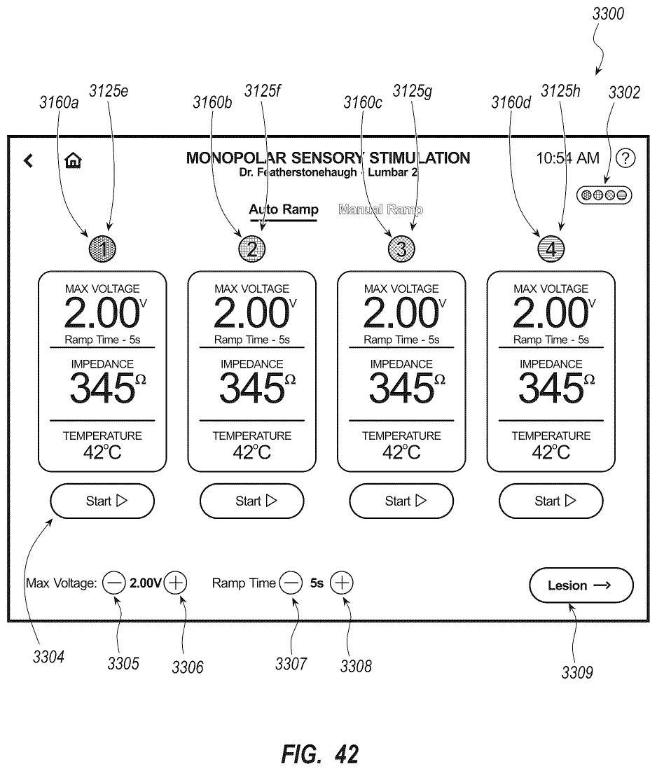

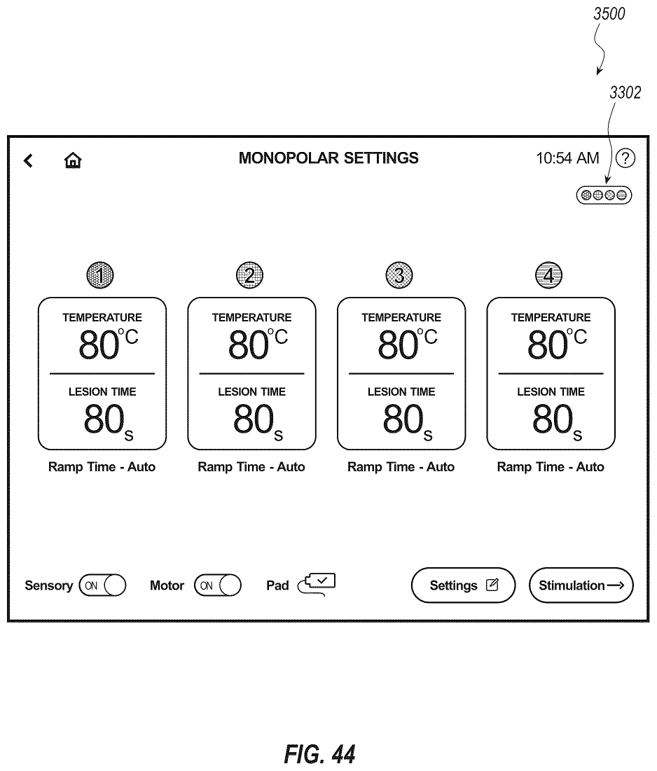

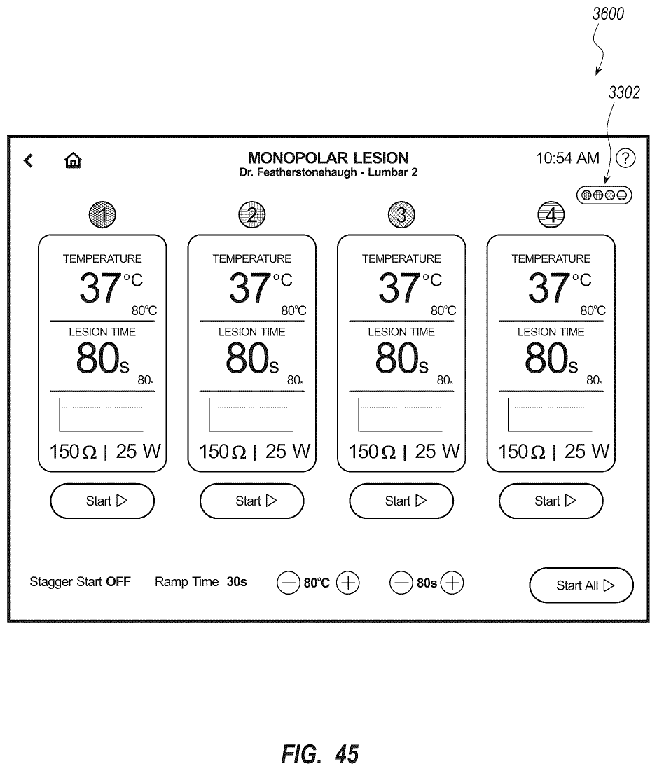

FIGS. 42-47 depict various screens that the controller 3109 can cause to be displayed individually, or one-at-a-time, on the touchscreen 3103. The screens may be shown in the order depicted in FIGS. 42-47 or in any other suitable order.

FIG. 42 depicts an illustrative control screen 3300 via which a monopolar sensory stimulation procedure is controllable, such as by way of inputs from a user via various virtual actuators 3304, 3305, 3306, 3307, 3308, 3309. The user can touch the touchscreen 3300 to actuate one or more of these actuators. In some instances, a user may desire to turn off lights that are displayed by the indicators 3125, 3145 during such a procedure.

In the illustrated embodiment, the controller 3109 causes a virtual actuator 3302, which may be a pressable virtual button, to appear on the control screen 3300. The virtual actuator 3302 may be present throughout the full time the control screen 3300 is displayed. Upon actuation of the virtual actuator 3302 by a user, the controller 3109 can cause the indicators 3125 of the ports 3114 and the indicators 3145 of the instrument assemblies 3104, where applicable, to cease displaying light. Subsequent actuation of the virtual actuator 3302 can reverse this action. That is, the controller 3109 can cause the indicators 3125 to display light again, such as differently colored light for port identification, as previously discussed.

The illustrated control screen 3300 can include further indicators 3125e-h on the screen that correlate or help identify the ports 3114a-d with which each is associated. Each indicator 3125e-h may be colored to match the port light color. Each indicator 3125e-h may further include a supplemental indicator 3160a-d, which in the present embodiment, is a numeral that matches a numeral displayed adjacent to each port 3114a-d.

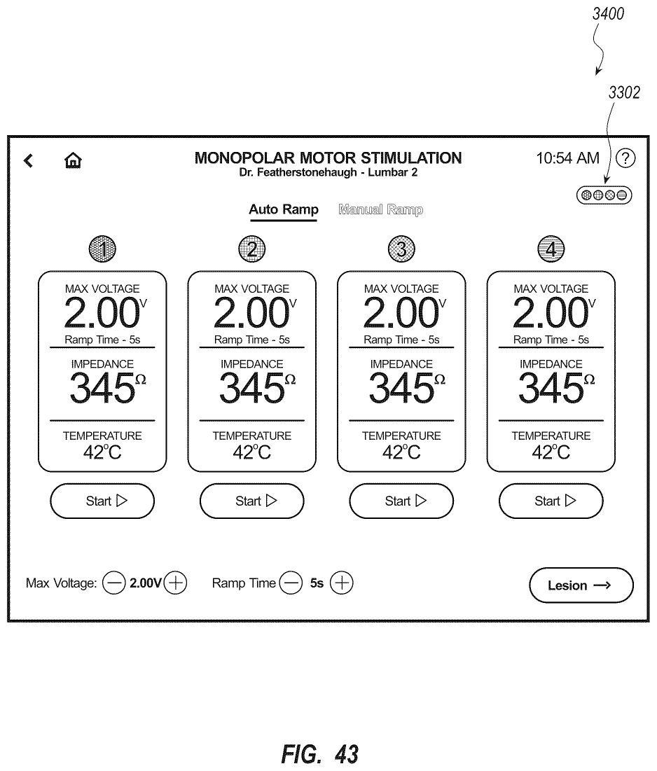

FIGS. 43-45 depict illustrative control screens 3400, 3500, 3600 via which conduction of a monopolar motor stimulation procedure, selection of settings for a monopolar procedure, or conduction of a monopolar lesion procedure are each controllable, respectively. The virtual actuator 3302 may be present on each control screen 3400, 3500, 3600 as well, for any suitable duration (including throughout an entirety of each display of each screen).

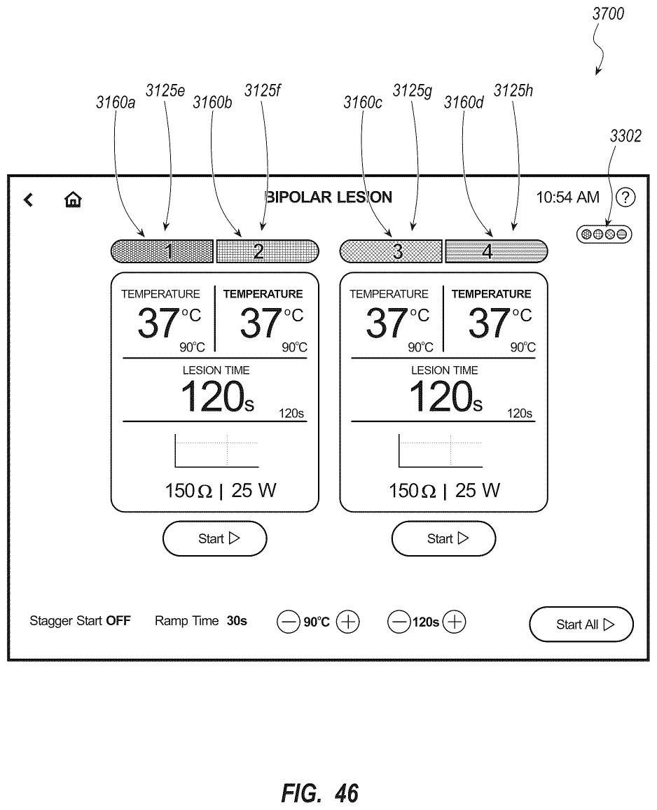

FIG. 46 depicts an illustrative control screen 3700 via which a bipolar lesion procedure is controllable. The control screen includes a different configuration of the indicators 3125e-h to provide intuitive understanding of a bipolar coupling of channels 1 and 2 and a further bipolar coupling of channels 3 of 4, while the supplemental indicators 3160a-d are unchanged. The virtual actuator 3302 may be present on the control screen 3700 as well, for any suitable duration (including throughout an entirety of the display of the control screen 3700).

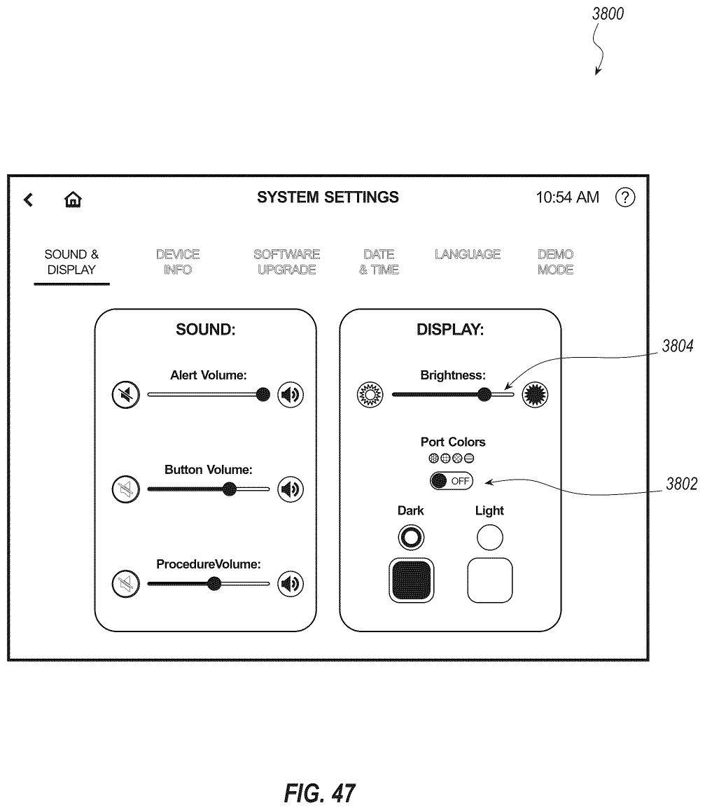

FIG. 47 depicts an illustrative control screen 3800 via which system settings can be selected and/or adjusted, including settings related to the indicators 3125a-d and/or the indicators 3145a-d (when coupled to the ports). The control screen 3800 includes a virtual actuator 3802 via which a user can select whether to make all ports display color of the same light, such as white, or to display differently colored lights. The controller 3109 can act accordingly in response to such actuation.

The control screen 3800 further includes a virtual actuator 3804 by which a brightness of the screen can be adjusted. In some embodiments, a similar virtual actuator 3804 may be present to adjust a brightness or intensity of light displayed by the indicators 3125a-d, 3145a-d.

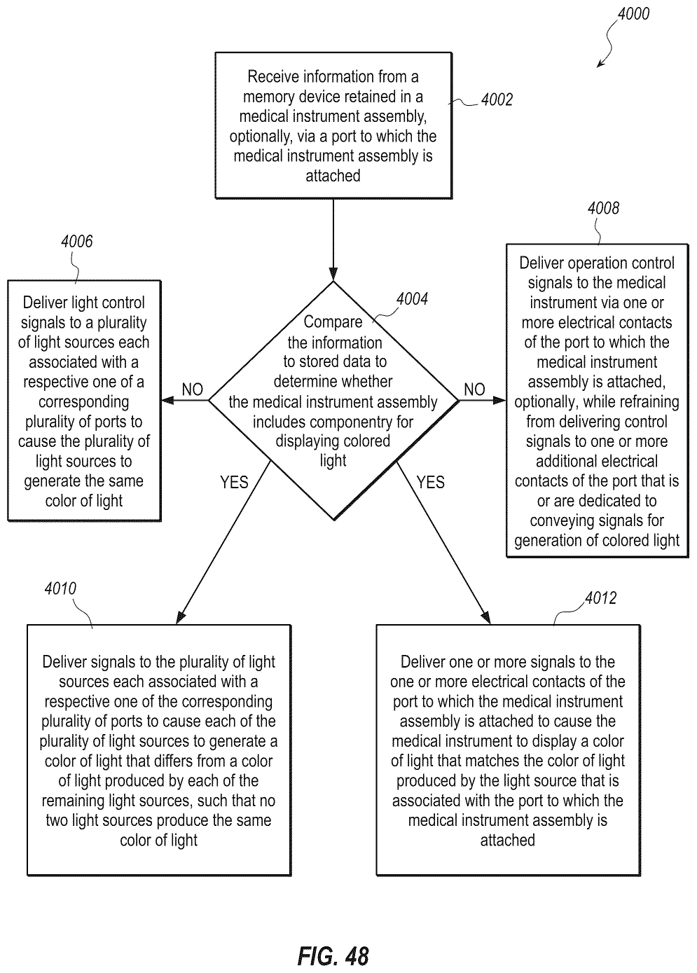

FIG. 48 is a diagram depicting operational stages of an illustrative method 4000 that can be conducted via embodiments of the system 3100 and/or other systems disclosed herein. In some embodiments, each step can be performed by the controller 3109.

At step 4002, the controller 3109 receives information from a memory device retained in a medical medical instrument assembly. In some instances, this information is received via a port to which the medical instrument assembly is attached.

At step 4004, the controller 3109 compares the information to stored data to determine whether the medical instrument assembly includes componentry for displaying colored light. The stored data may be stored in any suitable memory, e.g., within the control machine. In some instances, the stored data may include a list of acceptable codes, model numbers, etc.

The controller 3109 can proceed to step 4006 and/or step 4008 if the medical instrument assembly does not include componentry for displaying colored light. This may be the result of obtaining information from the instrument assembly indicating that the instrument assembly does not include an indicator. In other or further instances, this may be the result of an attempt to obtain such information from the instrument assembly, but not being able to obtain the information. For example, as previously discussed, some embodiments of instrument assemblies do not included memory devices for storing such information.

At step 4006, the controller 3109 delivers light control signals to a plurality of light sources each associated with a respective one of a corresponding plurality of ports to cause the plurality of light sources to generate the same color of light. In some instances, the color of light is white, although other options can be suitable. This may still allow for the ports to be lit up to assist with coupling of devices thereto and/of for lighting up supplemental indicators, such as the numerals associated with each port.

At step 4008, the controller 3109 delivers operation control signals to the medical instrument assembly via one or more electrical contacts of the port to which the medical instrument assembly is attached. In further instances, the controller 3109 does so while refraining from delivering control signals to one or more additional electrical contacts of the port that is or are dedicated to conveying signals for generation of colored light.

If the comparison at step 4004 is positive, the controller 3109 can proceed to one or more of steps 4010 or 4012.

At step 4010, the controller 3109 delivers signals to the plurality of light sources each associated with a respective one of the corresponding plurality of ports to cause each of the plurality of light sources to generate a color of light that differs from a color of light produced by each of the remaining light sources, such that no two light sources produce the same color of light.

At step 4012, the controller 3109 delivers one or more signals to the one or more electrical contacts of the port to which the medical instrument assembly is attached to cause the medical instrument to display a color of light that matches the color of light produced by the light source that is associated with the port to which the medical instrument assembly is attached.

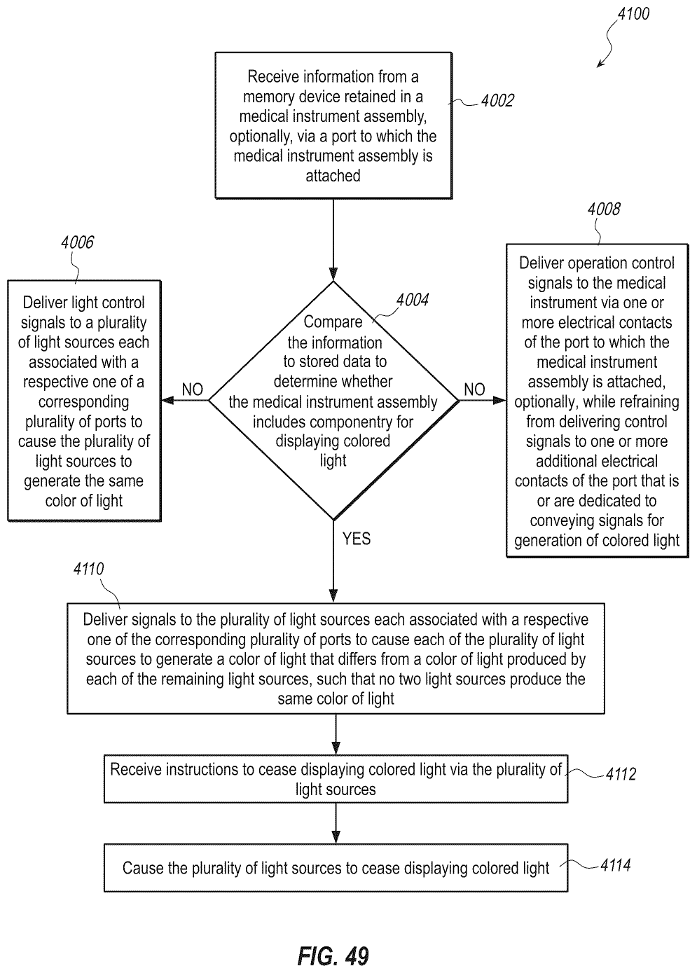

FIG. 49 is a diagram depicting operational stages of another illustrative method 4100 that can be conducted via embodiments of the system 3100 and/or other systems disclosed herein. In some embodiments, each step can be performed by the controller 3109. In some instances, the method includes one or more of the steps 4002, 4004, 4006, and/or 4008 previously described.

If the determination at step 4004 is positive, the controller 3109 can proceed to step 4110 and deliver signals to the plurality of light sources each associated with a respective one of the corresponding plurality of ports to cause each of the plurality of light sources to generate a color of light that differs from a color of light produced by each of the remaining light sources, such that no two light sources produce the same color of light.

At step 4112, the controller 3109 can receive instructions to cease displaying colored light via the plurality of light sources, such as may result from a user actuating a virtual actuator as previously discussed.

At step 4114, the controller 3109 can cause the plurality of light sources to cease displaying the colored light.

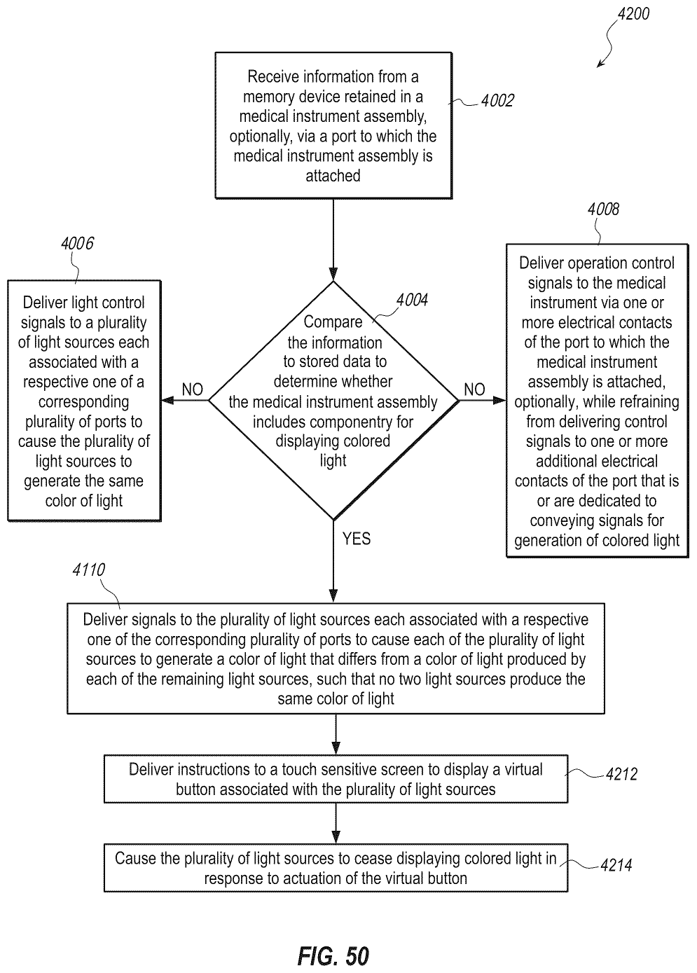

FIG. 50 is a diagram depicting operational stages of another illustrative method 4200 that can be conducted via embodiments of the system 3100 and/or other systems disclosed herein. In some embodiments, each step can be performed by the controller 3109. In some instances, the method includes one or more of the steps 4002, 4004, 4006, 4008, and/or 4110 previously described.

At step 4212, the controller 3109 can deliver instructions to a touch sensitive screen to display a virtual button associated with the plurality of light sources. At step 4214, the controller 3109 can cause the plurality of light sources to cease displaying colored light in response to actuation of the virtual button.

Other or further illustrative methods use a system that includes a control machine and multiple instruments couplable thereto, of which some may have indicators to convey information regarding which port to which the instrument has been coupled, and others may have no such indicator.

In some embodiments, a system can include a multicolor RGB LED mounted in the hub of an RF probe. The system can further include a probe connector that contains an RFID tag. The system can further include an RF generator connector that mates with the probe connector. In some embodiments, the RF generator connector is built into a module that contains an RFID reader/writer and also has a multicolor RGB LED and light pipe that illuminates the outside of the RF generator connector. In some embodiments, software running on the generator controls the lighting of the RF generator connector and probe LEDs.

In operation, the generator assigns each port a designated color. In some instances, color assignment can be dynamically controlled by the user in a software interface. The information for each port on the screen can include some indicator that is the same color as the port. The physical port has a light pipe area around the port that lights up to that port's specified color. When a probe is plugged into the port, the RFID reader/writer at that port writes a value to the RFID tag in the probe connector, which translates to a specific color that matches the color of the generator port. Circuitry in the probe would then lights up the LED in the probe hub to match the color designated by the RFID tag. In various embodiments, the LED controller can be in the probe itself or in the probe connector.

Other embodiments do not employ RFID readers or tags. For example, in some embodiments, the generator port and the probe connector that couples therewith can have extra electrodes/pins. The generator itself can include circuitry that can control a color of an RGB LED array within the probe. All of the decisions and processing take place in the generator, which sends the proper signal to the LED unit in the probe via the probe connector. In other or further embodiments, a different wireless protocol may be used to communicate between a transceiver at a port and/or a receiver or transceiver within an instrument assembly.

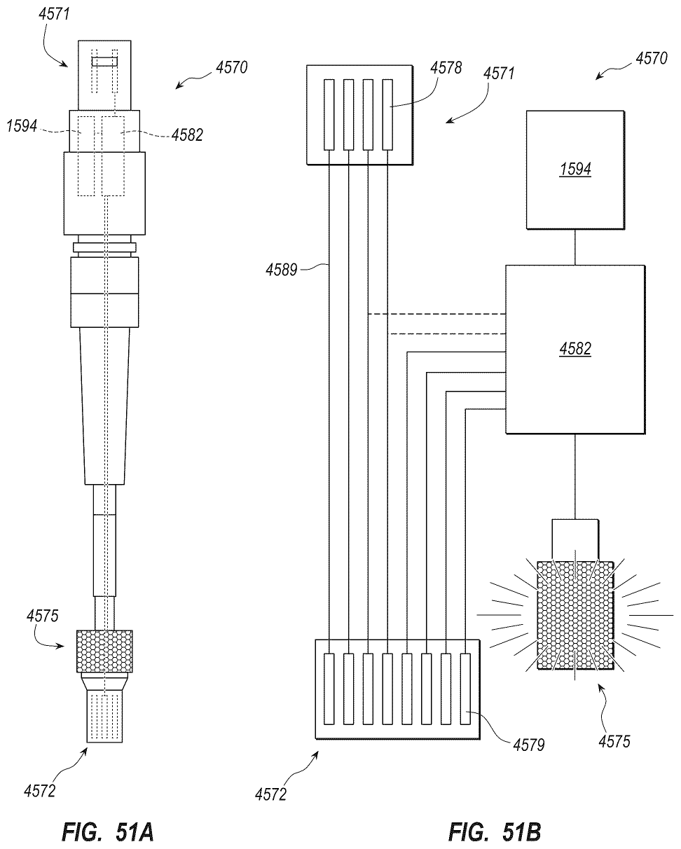

FIG. 51A is an elevation view of an embodiment of an adapter 4570 that is connectable at one end with a port of certain embodiments of control machines and is connectable at another end with embodiments of an instrument assembly, such as, for example, any of the instrument assemblies 3104a-d and, in further instances, embodiments of the instrument assemblies 3104e and 3014f.

FIG. 51B is a schematic diagram depicting various components of the adapter 4570. Like components are numbered according to like features in manners such as previously discussed. The adapter 4570 includes a distal connector 4572 that includes appropriate electrical contacts 4579 for coupling with the connector 3130 previously discussed.

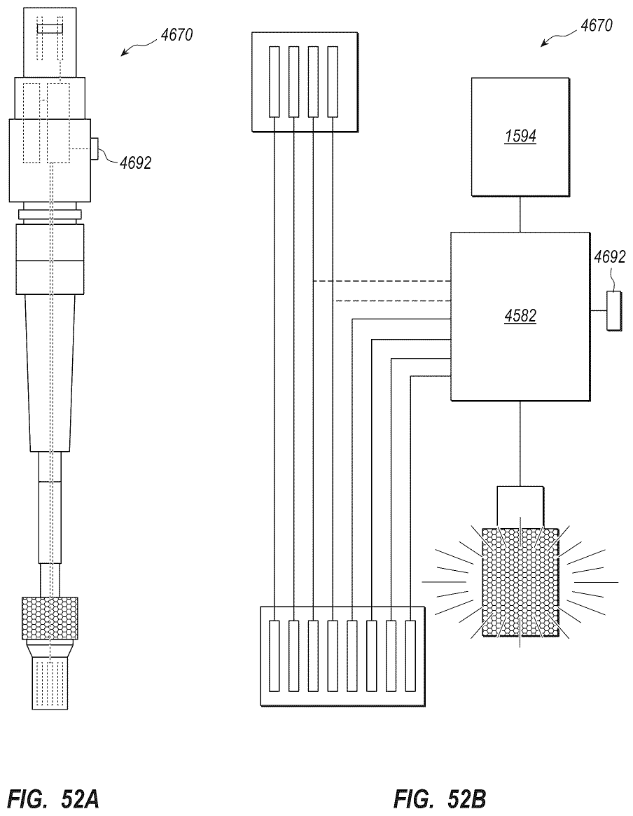

FIGS. 52A and 52B similarly depict an adapter 4670 that has been numbered according to the conventions previously discussed. This adapter differs from the adapter 4570 solely in its inclusion of an actuator 4692, which can function similarly to other actuators for adapters discussed elsewhere herein.

In further embodiments a large portion, or even an entirety, of an instrument assembly cable can light up, such as by using a clear or translucent jacket and a plurality of LEDs embedded throughout the cable length.

In some embodiments, the cable and/or the RF probe hub lights up in a specified color (e.g., yellow or red) during events that produce a warning or caution on that channel. In other or further embodiments, the lights may blink when there is an error.

In certain embodiments in which the user is capable of selecting what color each port is represented by, an on-screen user interface can be updated to match the selected color.

In some embodiments, an optical fiber is embedded in the cable to light up the whole cable with one LED or, variably, with a single LED assembly.

In some embodiments, the cable lights up in a manner that simulates flow in the direction of the patient when energy is being distributed from that port.

With reference to previous discussions, in some embodiments, any portion of the hub 137a may be transparent or translucent so as to light up from the LED unit 144a, with or without a dedicated light guide 146a.

In some embodiments, the control machine 101 may communicate wirelessly with the medical devices that are coupled to the respective ports.

In various embodiments, a user can selectively turn the correlating lights of the ports and instruments on and off.

In some embodiments, a port may illuminate only when a connected status with an instrument assembly has been achieved. In other embodiments, the port may be illuminated whenever a port is available for use, and may be illuminated continuously through a period before, during, and after connection of an instrument assembly therewith.

It is noted that many references have been made to electrical contacts and electrical communication. Other communication mechanisms are contemplated. For example, in various embodiments, communication among various components may be achieved by optical fibers and optical connections.

While much of the foregoing disclosure is presented in the context of radiofrequency neurotomy, the inventive concepts described herein are not limited to this specific field of endeavor. In particular, after conception and development of the inventive concepts detailed above, the inventors have determined that certain embodiments can be implemented in the fields of RF ablation more generally, and further, can be implemented in the more general field of ablation. Moreover, any suitable medical context in which multiple instruments may desirably be identified to determine their connection configuration is contemplated. Illustrative examples of medical contexts in which it may be helpful to determine coupling arrangements of multiple instruments include ultrasound delivery and/or diagnostic systems, cardiology systems, patient monitoring systems, drug delivery systems, or any other suitable multi-instrument medical, surgical, or diagnostic procedure.

Moreover, upon still further consideration, the inventors have determined that certain embodiments may be used in contexts outside of the medical field. For example, some embodiments may be used in the context of audio and/or video equipment, such as, by way of illustration: identifying multiple microphones or other audio input devices connected to a mixing board; identifying multiple devices (e.g., displays) connected, such as via HDMI cables, to a content device or a splitter; identifying multiple speakers connected to a receiver, etc. Other or further contexts can include connection of multiple electrical cables in other contexts, such as, for example, identifying multiple cables connected to an internet router, etc. Other contexts include, for example, industrial applications, such as in plumbing, construction, manufacturing, etc.

### EXAMPLES

The present paragraph recites illustrative examples of systems, kits, and methods that correspond with various embodiments of the foregoing written description and/or the illustrative drawings. In these examples, the terminology “Example X to Example Y” means Example X through Example Y, and thus includes the endpoints of the recited range of examples.

Example 1. A system comprising:
an assembly that comprises:
   a connector;
   a medical instrument in electrical communication with the connector;
   an instrument indicator; and
   a memory device comprising stored information; and
a control machine that comprises:
   a first port comprising a first port indicator;
   a second port comprising a second port indicator; and
   a controller in communication with each of the first and second ports, wherein the controller is configured to, when the connector of the assembly is attached to the first port:
      obtain the stored information from the memory device;
      make a determination based on the stored information that the assembly comprises the instrument indicator;
      in response to said determination, control the first port indicator to display light of a first color and control the second port indicator to display light of a second color that is different from the first color;
      control the instrument indicator to display light of the first color; and
      control the medical instrument to perform a function for treatment of a patient.

Example 2. The system of Example 1, wherein the controller is further configured to control the first and second port indicators to continue displaying light of the first and second colors, respectively, throughout said control of the medical instrument to perform the function for treatment of a patient.

Example 3. The system of Example 1, wherein the controller is further configured to, when the connector of the assembly is subsequently detached from the first port, control the first and second port indicators to simultaneously display a third color that differs from each of the first and second colors.

Example 4. The system of Example 1, further comprising an additional assembly that comprises:
an additional connector;
an additional medical instrument in electrical communication with the additional connector; and
an additional instrument indicator,
wherein the controller is further configured to, while the connector of the assembly remains attached to the first port and when the additional connector of the additional assembly is attached to the second port, control the additional instrument indicator to display light of the second color.

Example 5. The system of Example 4, wherein the controller is further configured to, while the additional connector is attached to the second port, control the additional medical instrument to perform an additional function for treatment of a patient.

Example 6. The system of Example 5, wherein the function performed by the medical instrument of the assembly and the additional function performed by the additional medical instrument of the additional assembly each comprises delivery of radiofrequency energy to the patient.

Example 7. The system of Example 1, further comprising a further assembly that comprises a further connector and a further medical instrument in electrical communication with the further connector, the further assembly being devoid of any instrument indicator,
wherein the controller is further configured to, after the connector of the assembly has been detached from the first port and the further connector of the further assembly has been attached to either the first port or the second port:
   make a determination that the further assembly is devoid of any instrument indicator; and
   in response to said determination, control the first and second port indicators to display light of a third color that differs from each of the first and second colors.

Example 8. The system of Example 7, wherein the third color is white.

Example 9. The system of Example 7, wherein the further assembly further comprises a further memory device comprising further stored information, and wherein the controller is configured to obtain the further stored information from the further memory device and make a determination that the further assembly is devoid of any instrument indicator based on the further stored information thus obtained.

Example 10. The system of Example 7, wherein the further assembly is devoid of any stored information indicative of an absence of an instrument indicator from the further assembly, and wherein the controller is further configured to:
attempt to obtain stored information indicative of an absence of an instrument indicator from the further assembly; and
make said determination that the further assembly is devoid of any instrument indicator based on an inability to obtain from the assembly any stored information indicative of said absence.

Example 11. The system of Example 1, further comprising an actuator in communication with the controller, wherein the controller is configured to, upon actuation of the actuator, cause the first and second port indicators to cease displaying light.

Example 12. The system of Example 11, wherein the controller is configured to, upon subsequent actuation of the actuator, cause the first and second port indicators to again display light of the first and second colors, respectively.

Example 13. The system of Example 11, wherein the control machine comprises a touchscreen, and wherein the controller causes the touchscreen to display the actuator as a virtual button in response to said determination that the assembly comprises the instrument indicator.

Example 14. The system of Example 13, wherein the control machine is configured to cause the touchscreen to display a plurality of different control screens on the touchscreen, and wherein the virtual button is present on at least multiple of said plurality of different control screens.

Example 15. The system of Example 1, wherein the memory device comprises a read/write device.

Example 16. The system of Example 1, wherein the memory device comprises a read only device.

Example 17. The system of Example 1, wherein the memory device comprises an electrically erasable programmable read-only memory (EEPROM) device.

Example 18. The system of Example 1, wherein the memory device comprises a radiofrequency identification (RFID) device.

Example 19. The system of Example 1, further comprising an elongated cable extending between the connector and the instrument.

Example 20. The system of Example 19, wherein the instrument indicator is physically secured to the assembly such that at least a portion of the instrument indicator is distal to a distal end of the elongated cable.

Example 21. The system of Example 19, wherein the instrument indicator is physically secured to the assembly such that at least a portion of the instrument indicator is closer to the medical instrument than to the connector.

Example 22. The system of Example 1, wherein the instrument indicator is physically secured to the assembly such that at least a portion of the instrument indicator is closer to the medical instrument than to the connector.

Example 23. The system of Example 1, wherein the medical instrument is configured to be positioned within the patient to perform the function for treatment of the patient.

Example 24. The system of Example 1, wherein the medical instrument comprises a radiofrequency electrode.

Example 25. The system of Example 24, further comprising a needle that is configured to be positioned within the patient, wherein the radiofrequency electrode is configured to be inserted into the needle to be physically and electrically coupled therewith.

Example 26. The system of Example 25, wherein, when the radiofrequency electrode is physically and electrically coupled with the needle, the controller is configured to deliver radiofrequency energy to the radiofrequency electrode and, thereby, to the needle.

Example 27. The system of Example 24, wherein the needle comprises a plurality of expandable tines.

Example 28. The system of Example 1, wherein the function for treatment of the patient comprises delivering radiofrequency energy to the patient.

Example 29. The system of Example 28, wherein the medical instrument is physically couplable with an additional medical instrument, and wherein said delivering radiofrequency energy to the patient comprises delivering the radiofrequency energy to the additional medical instrument.

Example 30. The system of Example 1, wherein the instrument indicator comprises a light-emitting diode array.

Example 31. The system of Example 30, wherein the light-emitting diode array is responsive to three separate channels of control signals.

Example 32. The system of Example 30, wherein the connector comprises three separate electrical contacts, each of which is separately coupled to the light-emitting diode array, and wherein the port comprises three separate electrical contacts that couple with the electrical contacts of the connector when the connector is attached to the port to permit the controller to control a color of light provided by the light-emitting diode array.

Example 33. The system of Example 1, wherein at least a portion of the instrument indicator is positioned within the medical instrument.

Example 34. The system of Example 1, wherein the medical instrument comprises a hub, and wherein the instrument indicator comprises a light-emitting diode array positioned within the hub of the medical instrument.

Example 35. The system of Example 1, wherein the connector comprises a plurality of electrical contacts via which the controller is configured to convey electrical control signals to the instrument indicator when the connector is attached to the port of the control machine.

Example 36. The system of Example 35, wherein the instrument indicator comprises a three-color array of light-emitting diodes, and wherein the controller is configured to separately control each color of light-emitting diodes via a separate one of the plurality of electrical contacts of the connector.

Example 37. The system of Example 35, wherein the connector comprises an additional electrical contact via which the controller provides power to the instrument indicator when the connector is attached to the port of the control machine.

Example 38. The system of Example 37, wherein the connector comprises a further electrical contact via which the controller delivers electrical signals to the medical instrument when the connector is attached to the port of the control machine.

Example 39. The system of Example 38, wherein the further electrical contact is configured to communicate radiofrequency energy from the controller to the medical instrument when the connector is attached to the port of the control machine.

Example 40. The system of Example 38, wherein the connector comprises a supplementary electrical contact that is electrically coupled with the memory device to permit communication between the controller and the memory device when the connector is attached to the port of the control machine.

Example 41. The system of Example 35, wherein the connector comprises a further electrical contact via which the controller delivers electrical signals to the medical instrument when the connector is attached to the port of the control machine.

Example 42. The system of Example 41, wherein the further electrical contact is configured to communicate radiofrequency energy from the controller to the medical instrument when the connector is attached to the port of the control machine.

Example 43. The system of Example 41, wherein the connector comprises a supplementary electrical contact that is electrically coupled with the memory device to permit communication between the controller and the memory device when the connector is attached to the port of the control machine.

Example 44. The system of Example 35, wherein the connector comprises a supplementary electrical contact that is electrically coupled with the memory device to permit communication between the controller and the memory device when the connector is attached to the port of the control machine.

Example 45. The system of Example 1, wherein the connector comprises a electrical contact that is electrically coupled with the memory device to permit communication between the controller and the memory device when the connector is attached to the port of the control machine.

Example 46. The system of Example 1, wherein the control machine comprises a radiofrequency generator.

Example 47. The system of Example 1, wherein the connector of the assembly is configured to attach to either the first port or the second port.

Example 48. The system of Example 47, wherein the controller is further configured to, when the connector of the assembly is detached from the first port and subsequently attached to the second port:
obtain the stored information from the memory device;
make a determination based on the stored information that the assembly comprises the instrument indicator;
in response to said determination, control the first port indicator to display light of the first color and control the second port indicator to display light of the second color; and
control the instrument indicator to display light of the second color.

Example 49. The system of Example 48, wherein the controller is further configured to cause the first and second port indicators to display light of a third color that differs from each of the first and second colors upon detachment of the connector from the first port and prior to attachment of the connector to the second port.

Example 50. A system comprising:
an assembly that comprises:
   a connector;
   an instrument;
   an elongated cable extending between the connector and the instrument;
   an instrument indicator; and
   a memory device comprising stored information; and
a control machine that comprises:
   a first port comprising a first port indicator;
   a second port comprising a second port indicator; and
   a controller in communication with each of the first and second ports, wherein the controller is configured to, when the connector of the assembly is attached to the first port:
      obtain the stored information from the memory device;
      make a determination based on the stored information that the assembly comprises the instrument indicator;
      in response to said determination, control the first port indicator to display light of a first color and control the second port indicator to display light of a second color that is different from the first color; and
      control the instrument indicator to display light of the first color.

Example 51. A system comprising:
an assembly that comprises a connector and a medical instrument and is devoid of any instrument indicator for displaying light;
a control machine that comprises:
   a first port comprising a first port indicator;
   a second port comprising a second port indicator; and
   a controller in communication with each of the first and second ports, wherein the controller is configured to, when the connector of the assembly is attached to the first port:
      attempt to obtain from the assembly stored information indicative of an absence from the assembly of any instrument indicator for displaying light;
      based on either the stored information successfully obtained from the assembly or an inability to obtain from the assembly any stored information indicative of said absence, make a determination that the assembly is devoid of any instrument indicator for displaying light;
      in response to said determination, control each of the first and second port indicators to display light of a first color; and
      control the medical instrument to perform a function for treatment of a patient.

Example 52. The system of Example 51, further comprising an additional assembly that comprises:
an additional connector;
an additional medical instrument;
an instrument indicator; and
a memory device comprising stored information;
wherein the controller is further configured to, while the connector of the assembly remains attached to the first port and when the additional connector of the additional assembly is attached to the second port:
   obtain the stored information from the memory device;
   make a determination based on the stored information that the additional assembly comprises the instrument indicator;
   in response to said determination, control the first port indicator to display light of a second color and control the second port indicator to display light of a third color, wherein the first, second, and third colors are all different from each other.

Example 53. The system of Example 52, wherein the controller is further configured to control the instrument indicator of the additional assembly to display light of the third color.

Example 54. The system of Example 52, wherein the controller is further configured to, upon detachment of the additional assembly from the second port, control each of the first and second port indicators to again display light of the first color.

Example 55. A system comprising:
an assembly that comprises:
   a connector;
   a medical instrument in electrical communication with the connector;
   an instrument indicator; and
   a memory device comprising stored information; and
a control machine that comprises:
   a first port comprising a first port indicator;
   a second port comprising a second port indicator; and
   a controller in communication with each of the first and second ports, wherein the controller is configured to, when the connector of the assembly is attached to the first port:
      obtain the stored information from the memory device;
      make a determination based on the stored information that the assembly comprises the instrument indicator;
      in response to said determination, control the first port indicator to display light of a first color and control the second port indicator to display light of a second color that is different from the first color;
      control the instrument indicator to display light of the first color; and
      control the medical instrument to perform a function for treatment of a patient.

Example 56. A system comprising:
an assembly that comprises:
   a connector;
   a medical instrument in electrical communication with the connector; and
   an instrument indicator; and
a control machine that comprises:
   a first port comprising a first port indicator;
   a second port comprising a second port indicator;
   an actuator; and
   a controller in communication with each of the first and second ports, wherein the controller is configured to, when the connector of the assembly is attached to the first port:
      control the first port indicator to display light of a first color and control the second port indicator to display light of a second color that is different from the first color; and
      upon actuation of the actuator, cause the first and second port indicators to cease displaying light.

Example 57. The system of Example 56, wherein the controller is further configured to control the instrument indicator to display light of the first color.

Example 58. The system of Example 56, wherein the controller is configured to, upon subsequent actuation of the actuator, cause the first and second port indicators to again display light of the first and second colors, respectively.

Example 59. The system of Example 56, wherein the control machine comprises a touchscreen, and wherein the controller causes the touchscreen to display the actuator as a virtual button upon determining that the assembly comprises the instrument indicator.

Example 60. The system of Example 59, wherein the control machine is configured to cause the touchscreen to display a plurality of different control screens on the touchscreen, and wherein the virtual button is present on at least multiple of said plurality of different control screens.

Example 61. The system of Example 56, wherein the actuator comprises a dedicated physical button.

Example 62. A system comprising:

an assembly that comprises:
a connector;
   a medical instrument in electrical communication with the connector;
   an instrument indicator; and
   a memory device comprising stored information; and
a control machine that comprises:
   a touchscreen;
   a first port comprising a first port indicator;
   a second port comprising a second port indicator; and
   a controller in communication with each of the first and second ports, wherein the controller is configured to, when the connector of the assembly is attached to the first port:
      obtain the stored information from the memory device;
      make a determination based on the stored information that the assembly comprises the instrument indicator;
      control the first port indicator to display light of a first color and control the second port indicator to display light of a second color that is different from the first color; and
      cause the touchscreen to display a virtual actuator configured for controlling one or more aspects of the first and second port indicators.

Example 63. The system of Example 62, wherein the controller is configured to, upon actuation of the actuator, cause the first and second port indicators to cease displaying light.

Example 64. The system of Example 63, wherein the controller is configured to, upon subsequent actuation of the actuator, cause the first and second port indicators to again display light of the first and second colors, respectively.

Example 65. The system of Example 62, wherein the controller is configured to, upon actuation of the actuator, cause the first and second port indicators to change an intensity of light displayed thereby.

Example 66. The system of Example 62, wherein the controller is further configured to control the instrument indicator to display light of the first color.

Example 67. The system of Example 62, wherein the control machine is configured to cause the touchscreen to display a plurality of different control screens on the touchscreen, and wherein the virtual actuator is present on at least multiple of said plurality of different control screens.

Example 68. A control machine comprising:
a first port comprising a first port indicator, the first port being configured to couple with a connector of a medical instrument assembly that comprises an instrument indicator and a memory device that contains stored information;
a second port comprising a second port indicator; and
a controller in communication with each of the first and second ports, wherein the controller is configured to, when the connector of the medical instrument assembly is attached to the first port:
   obtain the stored information from the memory device;
   make a determination based on the stored information that the assembly comprises the instrument indicator; and
   in response to said determination, control the first port indicator to display light of a first color and control the second port indicator to display light of a second color that is different from the first color.

Example 69. The control machine of Example 68, wherein the controller is further configured to control the instrument indicator of the medical instrument assembly to display light of the first color.

Example 70. The control machine of Example 69, wherein the second port is configured to couple with an additional medical instrument assembly that comprises an additional connector and an additional instrument indicator, and wherein the controller is further configured to, while the connector of the assembly remains attached to the first port and when the additional connector of the additional assembly is attached to the second port, control the additional instrument indicator to display light of the second color.

Example 71. The control machine of Example 68, wherein the controller is further configured to control the medical instrument assembly to perform a function for treatment of a patient.

Example 72. The control machine of Example 68, wherein the controller is further configured to control the first and second port indicators to continue displaying light of the first and second colors, respectively, while simultaneously controlling the medical instrument assembly to perform a function for treatment of a patient.

Example 73. The control machine of Example 68, wherein the controller is further configured to, when the connector of the medical instrument assembly is subsequently detached from the first port, control the first and second port indicators to simultaneously display a third color that differs from each of the first and second colors.

Example 74. The control machine of Example 68, further comprising a further assembly that comprises a further connector and a further medical instrument in electrical communication with the further connector, the further assembly being devoid of any instrument indicator,
wherein the controller is further configured to, after the connector of the assembly has been detached from the first port and the further connector of the further assembly has been attached to either the first port or the second port:
   make a determination that the further assembly is devoid of any instrument indicator; and
   in response to said determination, control the first and second port indicators to display light of a third color that differs from each of the first and second colors.

Example 75. The control machine of Example 74, wherein the further assembly further comprises a further memory device comprising further stored information, and wherein the controller is configured to obtain the further stored information from the further memory device and make a determination that the further assembly is devoid of any instrument indicator based on the further stored information thus obtained.

Example 76. The control machine of Example 74, wherein the further assembly is devoid of any stored information indicative of an absence of an instrument indicator from the further assembly, and wherein the controller is further configured to:
attempt to obtain stored information indicative of an absence of an instrument indicator from the further assembly; and
make said determination that the further assembly is devoid of any instrument indicator based on an inability to obtain from the assembly any stored information indicative of said absence.

Example 77. The control machine of Example 68, further comprising an actuator in communication with the controller, wherein the controller is configured to, upon actuation of the actuator, cause the first and second port indicators to cease displaying light.

Example 78. The control machine of Example 77, wherein the controller is configured to, upon subsequent actuation of the actuator, cause the first and second port indicators to again display light of the first and second colors, respectively.

Example 79. The control machine of Example 77, wherein the control machine comprises a touchscreen, and wherein the controller causes the touchscreen to display the actuator as a virtual button in response to said determination that the assembly comprises the instrument indicator.

Example 80. The control machine of Example 79, wherein the control machine is configured to cause the touchscreen to display a plurality of different control screens on the touchscreen, and wherein the virtual button is present on at least multiple of said plurality of different control screens.

Example 81. The control machine of Example 68, wherein the memory device comprises an electrically erasable programmable read-only memory (EEPROM) device, and wherein the controller is configured to read from and write to the EEPROM device.

Example 82. The control machine of Example 68, wherein the memory device comprises a radiofrequency identification (RFID) device and the control machine further comprises an RFID reader configured to communicate with the RFID device.

Example 83. The control machine of Example 68, wherein each of the first port and the second port is configured to separately attach to the connector of the medical instrument assembly.

Example 84. The control machine of Example 83, wherein the controller is further configured to, when the connector of the medical instrument assembly is detached from the first port and subsequently attached to the second port:
obtain the stored information from the memory device;
make a determination based on the stored information that the medical instrument assembly comprises the instrument indicator;
in response to said determination, control the first port indicator to display light of the first color and control the second port indicator to display light of the second color; and control the instrument indicator to display light of the second color.

Example 85. The control machine of Example 84, wherein the controller is further configured to cause the first and second port indicators to display light of a third color that differs from each of the first and second colors upon detachment of the connector from the first port and prior to attachment of the connector to the second port.

Example 86. The control machine of Example 68, wherein the control machine comprises a radiofrequency generator.

Example 87. A control machine comprising:
a first port comprising a first port indicator, the first port being configured to couple with a connector of an assembly that comprises an instrument indicator and a memory device that contains stored information;
a second port comprising a second port indicator; and
a controller in communication with each of the first and second ports, wherein the controller is configured to, when the connector of the assembly is attached to the first port:
   obtain the stored information from the memory device;
   make a determination based on the stored information that the assembly comprises the instrument indicator; and
   in response to said determination, control the first port indicator to display light of a first color and control the second port indicator to display light of a second color that is different from the first color.

Example 88. The control machine of Example 87, wherein the second port is configured to separately couple with the connector of the assembly.

Example 89. The control machine of Example 87, wherein the controller is further configured to control the instrument indicator to display light of the first color when the connector of the assembly is attached to the first port.

Example 90. A control machine comprising:
a first port comprising a first port indicator, the first port being configured to couple with a connector of a medical instrument assembly that is devoid of any instrument indicator for displaying light;
a second port comprising a second port indicator; and
a controller in communication with each of the first and second ports, wherein the controller is configured to, when the connector of the medical instrument assembly is attached to the first port:
   attempt to obtain from the medical instrument assembly stored information indicative of an absence from the medical instrument assembly of any instrument indicator for displaying light;
   based on either the stored information successfully obtained from the medical instrument assembly or an inability to obtain from the medical instrument assembly any stored information indicative of said absence, make a determination that the medical instrument assembly is devoid of any instrument indicator for displaying light; and
   in response to said determination, control each of the first and second port indicators to display light of a first color.

Example 91. The control machine of Example 90, wherein the controller is further configured to control the medical instrument assembly to perform a function for treatment of a patient while controlling each of the first and second port indicators to display light of the first color.

Example 92. The control machine of Example 90, wherein the second port is configured to couple with a connector of an additional medical instrument assembly that comprises an instrument indicator and a memory device comprising stored information, and
wherein the controller is further configured to, while the connector of the assembly remains attached to the first port and when the additional connector of the additional medical instrument assembly is attached to the second port:
   obtain the stored information from the memory device;
   make a determination based on the stored information that the additional medical instrument assembly comprises the instrument indicator;
   in response to said determination, control the first port indicator to display light of a second color and control the second port indicator to display light of a third color, wherein the first, second, and third colors are all different from each other.

Example 93. The control machine of Example 92, wherein the controller is further configured to control the instrument indicator of the additional medical instrument assembly to display light of the third color.

Example 94. The control machine of Example 92, wherein the controller is further configured to, upon detachment of the additional medical instrument assembly from the second port, control each of the first and second port indicators to again display light of the first color.

Example 95. A control machine comprising:
a first port comprising a first port indicator, the first port being configured to couple with a connector of a medical instrument assembly that comprises an instrument indicator and a memory device that comprises stored information;
a second port comprising a second port indicator; and
a controller in communication with each of the first and second ports, wherein the controller is configured to, when the connector of the medical instrument assembly is attached to the first port:
   obtain the stored information from the memory device;
   make a determination based on the stored information that the assembly comprises the instrument indicator;
   in response to said determination, control the first port indicator to display light of a first color and control the second port indicator to display light of a second color that is different from the first color;
   control the instrument indicator to display light of the first color; and
   control the medical instrument assembly to perform a function for treatment of a patient.

Example 96. A control machine comprising:
a first port comprising a first port indicator;
a second port comprising a second port indicator;
an actuator; and
   a controller in communication with each of the first and second ports, wherein the controller is configured to:
   control the first port indicator to display light of a first color and control the second port indicator to display light of a second color that is different from the first color; and upon actuation of the actuator, cause the first and second port indicators to cease displaying light.

Example 97. The control machine of Example 96, wherein the first port is configured to couple with a connector of a medical instrument assembly that comprises an instrument indicator, and wherein the controller is further configured to control the instrument indicator to display light of the first color.

Example 98. The control machine of Example 96, wherein the controller is configured to, upon subsequent actuation of the actuator, cause the first and second port indicators to again display light of the first and second colors, respectively.

Example 99. The control machine of Example 96, wherein the control machine comprises a touchscreen, and wherein the controller causes the touchscreen to display the actuator as a virtual button upon determining that the assembly comprises the instrument indicator.

Example 100. The control machine of Example 99, wherein the control machine is configured to cause the touchscreen to display a plurality of different control screens on the touchscreen, and wherein the virtual button is present on at least multiple of said plurality of different control screens.

Example 101. The control machine of Example 96, wherein the actuator comprises a dedicated physical button.

Example 102. A control machine comprising:
a touchscreen;
a first port comprising a first port indicator, the first port being configured to couple with a connector of a medical instrument assembly that comprises an instrument indicator and a memory device that comprises stored information;
a second port comprising a second port indicator; and
a controller in communication with each of the first and second ports, wherein the controller is configured to, when the connector of the medical instrument assembly is attached to the first port:
   obtain the stored information from the memory device;
   make a determination based on the stored information that the assembly comprises the instrument indicator;
   control the first port indicator to display light of a first color and control the second port indicator to display light of a second color that is different from the first color; and
   cause the touchscreen to display a virtual actuator configured for controlling one or more aspects of the first and second port indicators.

Example 103. The control machine of Example 102, wherein the controller is configured to, upon actuation of the actuator, cause the first and second port indicators to cease displaying light.

Example 104. The control machine of Example 103, wherein the controller is configured to, upon subsequent actuation of the actuator, cause the first and second port indicators to again display light of the first and second colors, respectively.

Example 105. The control machine of Example 102, wherein the controller is configured to, upon actuation of the actuator, cause the first and second port indicators to change an intensity of light displayed thereby.

Example 106. The control machine of Example 102, wherein the controller is further configured to control the instrument indicator to display light of the first color.

Example 107. The control machine of Example 102, wherein the control machine is configured to cause the touchscreen to display a plurality of different control screens on the touchscreen, and wherein the virtual actuator is present on at least multiple of said plurality of different control screens.

Example 108. An assembly comprising:
a connector configured to attach to a port of a control machine that comprises a port indicator associated with the port;
a medical instrument configured to perform a function for treatment of a patient when the connector is attached to the port of the control machine;
an instrument indicator configured to display a visually perceptible indicium that corresponds with a visually perceptible indicium displayed by the port indicator when the connector is attached to the port; and
a memory device comprising stored information for delivery to the control machine to indicate to the control machine that the medical apparatus includes the indicator.

Example 109. The assembly of Example 108, wherein the instrument indicator comprises a light source and is configured to be controlled by the control machine to display a color of light via the light source that matches a color of light displayed by the port indicator.

Example 110. The assembly of Example 108, wherein the memory device comprises a read/write device.

Example 111. The assembly of Example 108, wherein the memory device comprises a read only device.

Example 112. The assembly of Example 108, wherein the memory device comprises an electrically erasable programmable read-only memory (EEPROM) device.

Example 113. The assembly of Example 108, wherein the memory device comprises a radiofrequency identification (RFID) device.

Example 114. The assembly of Example 108, further comprising an elongated cable extending between the connector and the instrument.

Example 115. The assembly of Example 114, wherein the instrument indicator is physically secured to the assembly such that at least a portion of the instrument indicator is distal to a distal end of the elongated cable.

Example 116. The assembly of Example 114, wherein the instrument indicator is physically secured to the assembly such that at least a portion of the instrument indicator is closer to the medical instrument than to the connector.

Example 117. The assembly of Example 108, wherein the instrument indicator is physically secured to the assembly such that at least a portion of the instrument indicator is closer to the medical instrument than to the connector.

Example 118. The assembly of Example 108, wherein the medical instrument is configured to be controlled by the control machine to perform the function for treatment of the patient when the connector is attached to the port.

Example 119. The assembly of Example 108, wherein the instrument indicator is configured to be controlled by the control machine to display the visually perceptible indicium when the connector is attached to the port.

Example 120. The assembly of Example 108, wherein the medical instrument is configured to be positioned within the patient to perform the function for treatment of the patient.

Example 121. The assembly of Example 108, wherein the medical instrument comprises a radiofrequency electrode.

Example 122. The assembly of Example 121, wherein the radiofrequency electrode is configured to be inserted into a needle that is positioned within the patient to perform the function for treatment of the patient.

Example 123. The assembly of Example 122, wherein the function for treatment of the patient comprises delivering radiofrequency energy to the needle.

Example 124. The assembly of Example 108, wherein the function for treatment of the patient comprises delivering radiofrequency energy to the patient.

Example 125. The assembly of Example 124, wherein the medical instrument is physically couplable with an additional medical instrument, and wherein said delivering radiofrequency energy to the patient comprises delivering the radiofrequency energy to the additional medical instrument.

Example 126. The assembly of Example 108, wherein the instrument indicator comprises a light-emitting diode array.

Example 127. The assembly of Example 126, wherein the light-emitting diode array is responsive to three separate channels of control signals.

Example 128. The assembly of Example 126, wherein the connector comprises three separate electrical contacts, each of which is separately coupled to the light-emitting diode array to permit the control machine to control a color of light provided by the light-emitting diode array when the connector is attached to the port.

Example 129. The assembly of Example 108, wherein at least a portion of the instrument indicator is positioned within the medical instrument.

Example 130. The assembly of Example 108, wherein the medical instrument comprises a hub, and wherein the instrument indicator comprises a light-emitting diode array positioned within the hub of the medical instrument.

Example 131. The assembly of Example 108, wherein the connector is in electrical communication with the instrument indicator.

Example 132. The assembly of Example 131, wherein the connector comprises a plurality of electrical contacts that are electrically connected to the instrument indicator, and wherein each of the plurality of electrical contacts is configured to convey electrical control signals from the control machine to the instrument indicator.

Example 133. The assembly of Example 132, wherein the instrument indicator comprises an array of light-emitting diodes of three different colors, and wherein each electrical contact of said plurality of electrical contacts is configured to communicate electrical control signals from the control machine to the instrument indicator to control operation of one or more light-emitting diodes of a separate one of the three different colors.

Example 134. The assembly of Example 132, wherein the connector comprises an additional electrical contact in electrical communication with the instrument indicator that is configured provide power to the instrument indicator when the connector is attached to the port of the control machine.

Example 135. The assembly of Example 134, wherein the connector comprises a further electrical contact electrically coupled with the medical instrument to provide an electrical pathway between the control machine and the medical instrument when the connector is attached to the port of the control machine.

Example 136. The assembly of Example 135, wherein the further electrical contact is configured to communicate radiofrequency energy from the control machine to the medical instrument when the connector is attached to the port of the control machine.

Example 137. The assembly of Example 135, wherein the connector comprises a supplementary electrical contact that is electrically coupled with the memory device to provide at least a portion of a communication channel between the control machine and the memory device when the connector is attached to the port of the control machine.

Example 138. The assembly of Example 132, wherein the connector comprises a further electrical contact that is electrically coupled with the medical instrument to provide an electrical pathway between the control machine and the medical instrument when the connector is attached to the port of the control machine.

Example 139. The assembly of Example 138, wherein the additional electrical contact is configured to communicate radiofrequency energy from the control machine to the medical instrument when the connector is attached to the port of the control machine.

Example 140. The assembly of Example 138, wherein the connector comprises a supplementary electrical contact that is electrically coupled with the memory device to provide at least a portion of a communication channel between the control machine and the memory device when the connector is attached to the port of the control machine.

Example 141. The assembly of Example 132, wherein the connector comprises a supplementary electrical contact that is electrically coupled with the memory device to provide at least a portion of a communication channel between the control machine and the memory device when the connector is attached to the port of the control machine.

Example 142. The assembly of Example 108, wherein the connector comprises an electrical contact that is electrically coupled with the memory device to provide at least a portion of a communication channel between the control machine and the memory device when the connector is attached to the port of the control machine.

Example 143. A system comprising:
the assembly of any of Example 108 through Example 142; and
the control machine.

Example 144. A method comprising:
obtaining stored information from a memory device of a medical instrument assembly that is attached to a port of a control machine;
based on the stored information, determining that the medical instrument assembly comprises an indicator;
responsive to said determining, controlling an indicator of the medial instrument assembly and an indicator associated with the port of the control machine to display light of a first color.

Example 145. The method of Example 144, wherein the control machine includes an additional port, and wherein the method further comprises controlling an additional indicator associated with the additional port to display light of a second color that differs from the first color.

Example 146. The method of Example 145, wherein a further medical instrument assembly that comprises a further indicator is attached to the additional port of the control machine, and wherein the method further comprises controlling the further indicator of the further medical instrument assembly to display light of the second color.

Example 147. The method of Example 144, wherein the control machine comprises a touchscreen, and wherein the method further comprises causing the touchscreen to display a virtual actuator responsive to said determining.

Example 148. The method of Example 147, further comprising, responsive to an actuation of actuator, causing each indicator of the ports and of the medical instrument assemblies to alter an amount of light displayed thereby.

Example 149. The method of Example 147, further comprising, responsive to actuation of an actuator, causing each indicator of the ports and of the medical instrument assemblies to cease displaying light.

Example 150. The method of Example 149, further comprising, responsive to a subsequent actuation of the actuator, causing each indicator of the ports and of the medical instrument assemblies to resume displaying light.

Example 151. The method of any of Example 144 through Example 150, wherein said obtaining, determining, and controlling is performed by a controller of the control machine.

Example 152. A method comprising:
attempting to obtain stored information from a medical instrument assembly that is attached to a first port of a control machine;
determining that the medical instrument assembly is devoid of any indicator for displaying light, based on either stored information successfully obtained from the medical instrument assembly or an inability to obtain from the medical instrument assembly any stored information indicative of a presence or absence of an indicator;
responsive to said determining, controlling a plurality of indicators to display light of the same color, wherein each indicator is individually associated with one port of a corresponding plurality of ports, wherein said plurality of ports includes the first port.

Example 153. A system comprising:
a medical instrument assembly configured to perform a function for treatment of a patient, the medical instrument assembly comprising an indicator configured to display a visually perceptible indicium;
a control machine comprising a first port; and
an adapter comprising:
   a proximal connector configured to couple with the first port of the control machine;
   a distal connector configured to connect to the medical instrument assembly; and
   an indicator configured to display a visually perceptible indicium,
wherein the adapter is, when the proximal connector is coupled with the first port of the control machine and the distal connector is connected to the medical instrument assembly, configured to:
   establish communication between the control machine and the medical instrument assembly to permit the control machine to control the function of the medical instrument assembly; and
   control one or more of the indicator of the adapter and the indicator of the medical instrument assembly to ensure that the visually perceptible indicia displayed by both indicators signify a correlation between the adapter and the medical instrument assembly.

Example 154. The system of Example 153, wherein:
the control machine includes one or more additional ports that, collectively with the first port, define a plurality of ports; and
the proximal connector of the adapter is configured to individually couple with any port of the plurality of ports such that, when coupled with said port and when the distal connector is connected to the medical instrument assembly, the control machine controls the function of the medical instrument assembly and the adapter controls one or more of the indicator of the adapter and the indicator of the medical instrument assembly to ensure that the visually perceptible indicia displayed by the indicators signify a correlation between the adapter and the medical instrument assembly.

The system of Example 154, further comprising: Example 155.
one or more additional medical instrument assemblies, each additional medical instrument assembly:
   being configured to perform a function for treatment of a patient; and
   comprising an indicator configured to display a visually perceptible indicium; and
one or more additional adapters, each additional adapter comprising:
   a proximal connector configured to individually couple with any port of said plurality of ports of the control machine;
   a distal connector configured to connect to any of the one or more additional medical instrument assemblies; and
   an indicator configured to display a visually perceptible indicium,
wherein each additional adapter is configured to, when the proximal connector thereof is coupled with any port of said plurality of ports of the control machine and the distal connector is connected to any of the one or more additional medical instrument assemblies:
   establish communication between the control machine and the additional medical instrument assembly that is coupled to the additional adapter to permit the control machine to control the function of the additional medical instrument assembly; and
   control one or more of the indicator of the additional adapter and the indicator of the additional medical instrument assembly that is coupled to the additional adapter to ensure that the visually perceptible indicia displayed by the indicators of the additional adapter and of the additional medical instrument assembly signify a correlation between the additional adapter and the additional medical instrument assembly.

Example 156. The system of Example 155, wherein each of the adapter and the one or more additional adapters is configured to control its respective indicator to ensure that the visual indicium displayed thereby is visually distinct from the visual indicium displayed by all other adapters.

Example 157. The system of Example 156, wherein the adapter comprises an actuator by which a user can manually control the visually perceptible indicium displayed by the indicator of the adapter.

Example 158. The system of Example 155, wherein the adapter comprises an actuator by which a user can manually control the visually perceptible indicium displayed by the indicator of the adapter.

Example 159. The system of Example 154, further comprising one or more additional medical instrument assemblies, each additional medical instrument assembly:
being configured to perform a function for treatment of a patient; and
comprising an indicator configured to display a visually perceptible indicium,
wherein the adapter comprises:
one or more additional proximal connectors, wherein the proximal connector and the additional proximal connectors of the adapter are configured to simultaneously couple with a corresponding number of said plurality of ports of the control machine;
one or more additional distal connectors associated with each of the one or more additional proximal connectors, each additional distal connector being configured to connect to any of the one or more additional medical instrument assemblies; and
one or more additional indicators associated with each of the one or more additional proximal connectors, each additional indicator configured to display a visually perceptible indicium,
wherein the adapter is configured to, for each proximal connector of the adapter coupled to a port of the control machine, when the additional distal connector associated therewith is connected to one of said one or more additional medical instrument assemblies:
establish communication between the control machine and the connected additional medical instrument assembly to permit the control machine to control the function of the connected additional medical instrument assembly; and
control one or more of the indicator of the additional adapter and the indicator of the additional medical instrument assembly coupled thereto to ensure that the visually perceptible indicia displayed by the indicators of the additional adapter and of the additional medical instrument assembly signify a correlation between the additional adapter and the additional medical instrument assembly.

Example 160. The system of Example 153, wherein the adapter comprises an actuator by which a user can manually control the visually perceptible indicium displayed by the indicator of the adapter.

Example 161. The system of Example 153, wherein the adapter comprises a controller that is configured to draw power from the control machine when the proximal connector is coupled with the port of the control machine.

Example 162. The system of Example 161, wherein the controller is configured to control said one or more of the indicator of the adapter and the indicator of the medical instrument assembly.

Example 163. The system of Example 153, wherein the adapter comprises an internal power source.

Example 164. The system of Example 163, wherein the internal power source comprises a battery.

Example 165. The system of Example 163, wherein the adapter further comprises a controller that is configured to draw power from the power source.

Example 166. The system of Example 165, wherein the controller is configured to control said one or more of the indicator of the adapter and the indicator of the medical instrument assembly.

Example 167. The system of Example 153, wherein the correlation between the adapter and the medical instrument assembly comprises a display of matching colors via the indicators.

Example 168. The system of Example 153, wherein the indicators are configured to display light.

Example 169. The system of Example 168, wherein each indicator is configured to display a color of light that is selected as one color from among a predetermined number of colors.

Example 170. The system of Example 153, wherein the adapter comprises a hub to which each of the proximal and distal connectors are attached.

Example 171. The system of Example 153, wherein the adapter comprises a flexible stem that extends between the proximal and distal connectors.

Example 172. An adapter configured for used with a medical instrument assembly that is configured to perform a function for treatment of a patient, the medical instrument assembly comprising an indicator configured to display a visually perceptible indicium, the adapter further being configured for use with a control machine that comprises a port, the adapter comprising:
a proximal connector configured to couple with the port of the control machine;
a distal connector configured to connect to the medical instrument assembly; and
an indicator configured to display a visually perceptible indicium,
wherein the adapter is configured to, when the proximal connector is coupled with the port of the control machine and the distal connector is connected to the medical instrument assembly:
establish communication between the control machine and the medical instrument assembly to permit the control machine to control the function of the medical instrument assembly; and
control one or more of the indicator of the adapter and the indicator of the medical instrument assembly to ensure that the visually perceptible indicia displayed by the indicators signify a correlation between the adapter and the medical instrument assembly.

Example 173. The adapter of Example 172, further comprising an actuator by which a user can manually control the visually perceptible indicium displayed by the indicator of the adapter.

Example 174. The adapter of Example 172, further comprising a controller that is configured to draw power from the control machine when the proximal connector is coupled with the port of the control machine.

Example 175. The adapter of Example 174, wherein the controller is configured to control said one or more of the indicator of the adapter and the indicator of the medical instrument assembly.

Example 176. The adapter of Example 172, wherein the adapter comprises an internal power source.

Example 177. The adapter of Example 176, wherein the internal power source comprises a battery.

Example 178. The adapter of Example 176, wherein the adapter further comprises a controller that is configured to draw power from the power source.

Example 179. The adapter of Example 179, wherein the controller is configured to control said one or more of the indicator of the adapter and the indicator of the medical instrument assembly.

Example 180. The adapter of Example 172, wherein the correlation between the adapter and the medical instrument assembly comprises a display of matching colors via the indicators.

Example 181. The adapter of Example 172, wherein the indicator of the adapter is configured to display light.

Example 182. The adapter of Example 181, wherein the indicator of the adapter is configured to display a color of light that is selected as one color from among a predetermined number of colors.

Example 183. The adapter of Example 172, wherein the adapter comprises a hub to which each of the proximal and distal connectors are attached.

Example 184. The adapter of Example 172, wherein the adapter comprises a flexible stem that extends between the proximal and distal connectors.

Example 185. A system comprising:
a control machine comprising:
   a first port;
   a first control machine indicator associated with the first port that is configured to display a first visual indicium;
   a second port; and
   a second control machine indicator associated with the second port that is configured to display a second visual indicium that conveys information distinct from the first visual indicium,
   the first port being configured to deliver a first signal representative of the first visual indicium and the second port being configured to deliver a second signal representative of the second visual indicium; and
a first instrument assembly comprising:
   a first connector configured to couple with either the first port or the second port of the control machine;
   an elongated first cable comprising a proximal end that is coupled with the first connector;
   a first instrument coupled with a distal end of the first cable; and
   a first instrument indicator associated with the first instrument, the first instrument indicator being configured to display a third visual indicium that is responsive to either the first signal or the second signal such that the third visual indicium corresponds with:
      the first visual indicium if the first connector is connected to the first port; or
      the second visual indicium if the first connector is connected to the second port.

Example 186. The system of Example 185, further comprising a second instrument assembly comprising:
a second connector configured to couple with either the first port or the second port of the control machine;
an elongated second cable comprising a proximal end that is coupled with the second connector;
a second instrument coupled with a distal end of the second cable; and
a second instrument indicator associated with the second instrument, the second instrument indicator being configured to display a fourth visual indicium that is responsive to either the first signal or the second signal such that the fourth visual indicium corresponds with:
   the first visual indicium if the second connector is connected to the first port; or
   the second visual indicium if the second connector is connected to the second port.

Example 187. The system of Example 185 or Example 186, wherein the control machine further comprises:
a third port; and
a third control machine indicator associated with the third port that is configured to display a fifth visual indicium that conveys information distinct from each of the first and second visual indicia,
wherein the first connector of the first instrument assembly is configured to couple with any one of the first, second, and third ports of the control machine, and
wherein the third visual indicium of the first instrument assembly is responsive to any one of the first, second, and third signals, such that the third visual indicium corresponds with:
   the first visual indicium if the first connector is connected to the first port;
   the second visual indicium if the first connector is connected to the second port; or the fifth visual indicium if the first connector is connected to the third port.

Example 188. The system of Example 186 or Example 187, wherein the second connector of the second instrument assembly is configured to couple with any one of the first, second, and third ports of the control machine, and
wherein the fourth visual indicium of the second instrument assembly is responsive to any one of the first, second, and third signals, such that the fourth visual indicium corresponds with:
   the first visual indicium if the second connector is connected to the first port;
   the second visual indicium if the second connector is connected to the second port; or
   the fifth visual indicium if the second connector is connected to the third port.

Example 189. The system of Example 187 or Example 188, further comprising a third instrument assembly comprising:
a third connector configured to couple with any one of the first, second, and third ports of the control machine;
an elongated third cable comprising a proximal end that is coupled with the third connector;
a third instrument coupled with a distal end of the third cable; and
a third instrument indicator associated with the third instrument, the third instrument indicator being configured to display a sixth visual indicium that is responsive any one of the first, second, and third signals such that the sixth visual indicium corresponds with:
   the first visual indicium if the third connector is connected to the first port;
   the second visual indicium if the third connector is connected to the second port; or
   the fifth visual indicium if the third connector is connected to the third port.

Example 190. The system of any one of Example 187 to Example 189, wherein the control machine further comprises:
a fourth port; and
a fourth control machine indicator associated with the fourth port that is configured to display a seventh visual indicium that conveys information distinct from each of the first, second, and fifth visual indicia,
wherein the first connector of the first instrument assembly is configured to couple with any one of the first, second, third, and fourth ports of the control machine, and
wherein the third visual indicium of the first instrument assembly is responsive to any one of the first, second, third, and fourth signals, such that the third visual indicium corresponds with:
   the first visual indicium if the first connector is connected to the first port;
   the second visual indicium if the first connector is connected to the second port;
   the fifth visual indicium if the first connector is connected to the third port; or
   the seventh visual indicium if the first connector is connected to the fourth port.

Example 191. The system of Example 189 or Example 190, further comprising a fourth instrument assembly comprising:
a fourth connector configured to couple with any one of the first, second, third, and fourth ports of the control machine;
an elongated fourth cable comprising a proximal end that is coupled with the fourth connector;
a fourth instrument coupled with a distal end of the fourth cable; and
a fourth instrument indicator associated with the fourth instrument, the fourth instrument indicator being configured to display an eight visual indicium that is responsive any one of the first, second, third, and fourth signals such that the eighth visual indicium corresponds with:
   the first visual indicium if the fourth connector is connected to the first port;
   the second visual indicium if the fourth connector is connected to the second port;
   the fifth visual indicium if the fourth connector is connected to the third port; or
   the seventh visual indicium if the fourth connector is connected to the fourth port.

Example 192. The system of any one of Example 185 to Example 191, wherein the first control machine indicator comprises a first light source.

Example 193. The system of Example 192, wherein the second control machine indicator comprises a second light source.

Example 194. The system of Example 192 or Example 193, wherein the first light source comprises a first light emitting diode.

Example 195. The system of Example 194 wherein the second light source comprises a second light emitting diode.

Example 196. The system of Example 194 or Example 195, wherein the first light source further comprises a first light guide that extends about the first port.

Example 197. The system of Example 196, wherein the second light source further comprises a second light guide that extends about the second port.

Example 198. The system of any one of Example 192 to Example 197, wherein the first visual indicium comprises light of a first color generated by the first light source.

Example 199. The system of Example 198, wherein the second visual indicium comprises light of a second color generated by the second light source, the second color being different from the first color.

Example 200. The system of any one of Example 185 to Example 199, wherein the first instrument indicator comprises a third light source.

Example 201. The system of Example 200, wherein the third light source comprises a third light emitting diode.

Example 202. The system of Example 200 or Example 201, wherein the third visual indicium comprises:
light of the first color if the first connector is connected to the first port; or
light of the second color if the first connector is connected to the second port.

Example 203. The system of any one of Example 185 to Example 202, wherein the first control machine indicator comprises a first permanent marking.

Example 204. The system of Example 203, wherein the second control machine indicator comprises a second permanent marking.

Example 205. The system of Example 203 or Example 204, wherein the first permanent marking comprises at least one of a symbol and a colored element.

Example 206. The system of Example 205, wherein the colored element comprises a colored ring that extends about the first port.

Example 207. The system of any one of Example 185 to Example 206, wherein the first control machine indicator comprises a first changeable element controllable by the control machine.

Example 208. The system of Example 207, wherein the second control machine indicator comprises a second changeable element controllable by the control machine.

Example 209. The system of Example 207 or Example 208, wherein the first changeable element comprises one or more of a dedicated lighting element, a dedicated display element, and a graphic element on a screen.

Example 210. The system of any one of Example 207 to Example 209, wherein the control machine is configured to selectively activate and deactivate the first changeable element.

Example 211. The system of any one of Example 207 to Example 210, wherein the control machine is configured to selectively change a value of the first visual indicium.

Example 212. The system of Example 211, wherein the first visual indicium comprises a colored light, and wherein changing the value of the first visual indicium comprise changing a color of the colored light.

Example 213. The system of Example 211 or Example 212, wherein the first visual indicium comprises a symbol, and wherein changing the value of the first visual indicium comprises changing the symbol to a different symbol.

Example 214. The system of any one of Example 185 to Example 213, wherein the first instrument indicator of the first instrument comprises a changeable element controllable by the control machine.

Example 215. The system of Example 214, wherein the changeable element comprises one or more of a lighting element and a display element.

Example 216. The system of Example 213 or Example 214, wherein the control machine is configured to selectively activate and deactivate the changeable element.

Example 217. The system of any one of Example 185 to Example 216, wherein correspondence between the third visual indicium and one of the first and second visual indicia comprises a color of the third visual indicium matching a color of said one of the first and second visual indicia.

Example 218. The system of any one of Example 185 to Example 217, wherein correspondence between the third visual indicium and one of the first and second visual indicia comprises a symbol of the third visual indicium matching a symbol of said one of the first and second visual indicia.

Example 219. The system of any one of Example 185 to Example 218, wherein the first instrument indicator is positioned on a hub of the first instrument.

Example 220. The system of any one of Example 185 to Example 219, wherein the first instrument assembly comprises a controller and the first instrument indicator comprises a light emitting diode configured to be controlled by the controller.

Example 221. The system of Example 220, wherein the controller is positioned at or adjacent to the first connector.

Example 222. The system of Example 220 or Example 221, wherein at least one communication line that is coupled to each of the controller and the light emitting diode extends through the first cable.

Example 223. The system of any one of Example 185 to Example 222, wherein at least one electrical line extends from the first connector through the first cable and is coupled with the first instrument indicator to power the first instrument indicator.

Example 224. The system of any one of Example 185 to Example 223, wherein the first port of the control machine comprises an RFID reader that is configured to generate the first signal that is representative of the first visual indicium.

Example 225. The system of Example 224, wherein the second port of the control machine comprises an RFID reader that is configured to generate the second signal that is representative of the second visual indicium.

Example 226. The system of Example 224 or Example 225, wherein the first instrument assembly further comprises an RFID tag that is configured to respond to the first signal that is representative of the first visual indicium, wherein the RFID tag is operatively coupled with the first instrument indicator.

Example 227. The system of Example 226, wherein the RFID tag is communicatively coupled with a controller, wherein the first instrument indicator comprises a light emitting diode, and wherein the controller is configured to control operation of the light emitting diode based on information received from the RFID tag responsive to first signal.

Example 228. The system of any one of Example 185 to Example 227, wherein the first instrument comprises a radiofrequency probe configured for use in radiofrequency neurotomy procedures.

Example 229. The system of 76, wherein the radiofrequency probe is configured to physically and electrically couple with a radiofrequency neurotomy needle.

Example 230. A control machine comprising:
a first port;
a first indicator associated with the first port that is configured to display a first visual indicium;
a first RFID reader configured to deliver a first signal representative of the first visual indicium to an instrument assembly when the instrument assembly is coupled with the first port;
a second port;
a second indicator associated with the second port that is configured to display a second visual indicium that conveys information distinct from the first visual indicium; and
a second RFID reader configured to deliver a second signal representative of the second visual indicium to the instrument assembly when the instrument assembly is coupled with the second port.

Example 231. An instrument assembly comprising:
a connector configured to couple with either a first port or a second port of a control machine that comprises a first control machine indicator associated with the first port that is configured to display a first visual indicium, a second control machine indicator associated with the second port that is configured to display a second visual indicium that conveys information distinct from the first visual indicium, the first port being configured to deliver a first signal representative of the first visual indicium and the second port being configured to deliver a second signal representative of the second visual indicium;
an elongated cable comprising a proximal end that is coupled with the first connector;
an instrument coupled with a distal end of the cable; and
an instrument indicator associated with the first instrument, the instrument indicator being configured to display a third visual indicium that is responsive to either the first signal or the second signal such that the third visual indicium corresponds with:
   the first visual indicium if the first connector is connected to the first port; or
   the second visual indicium if the first connector is connected to the second port.

Additional embodiments capable of derivation from the preceding independent and dependent Examples are also expressly incorporated into the present written description. These additional embodiments are determined by replacing the dependency of a given dependent Example with the phrase “any one of Examples [x] through the immediately preceding Example,” where the bracketed term “[x]” is replaced with the number of the most recently recited independent Example. For example, for the first set of Examples that begins with independent Example 1, Example 3 can depend from either of Examples 1 and 2, with these separate dependencies yielding two distinct embodiments; Example 4 can depend from any one of Examples 1, 2, or 3, with these separate dependencies yielding three distinct embodiments; Example 5 can depend from any one of Examples 1, 2, 3, or 4, with these separate dependencies yielding four distinct embodiments; and so on.

Any suitable combination of the various features of the various embodiments disclosed herein is contemplated. Moreover, any suitable combination of the various features of the various embodiments disclosed herein and those of the aforementioned U.S. patent applications are also contemplated.

The term “coupled to” can mean connected to in any suitable fashion, whether that coupling is direct or indirect, wired or wireless, etc. Separate components may be coupled to each other. Moreover, in some instances, where separately identified components are integrally formed from a unitary piece of material, or stated otherwise, are included together in a monolithic element, those elements may also be said to be coupled to one another.

Although the foregoing detailed description contains many specifics for the purpose of illustration, a person of ordinary skill in the art will appreciate that many variations and alterations to the following details can be made and are considered to be included herein. Accordingly, the foregoing embodiments are set forth without any loss of generality to, and without imposing limitations upon, any claims set forth. It is also to be understood that the terminology used herein is for the purpose of describing particular embodiments only, and is not intended to be limiting. Unless defined otherwise, all technical and scientific terms used herein have the same meaning as commonly understood by one of ordinary skill in the art to which this disclosure belongs.

Recitation in the claims of the term “first” with respect to a feature or element does not necessarily imply the existence of a second or additional such feature or element. Embodiments of the invention in which an exclusive property or privilege is claimed are defined as follows.

1. A system comprising:
an assembly that comprises:
a connector;
a medical instrument in electrical communication with the connector;
an instrument indicator; and
a memory device comprising stored information; and
a control machine that comprises:
a first port comprising a first port indicator;
a second port comprising a second port indicator; and
a controller in communication with each of the first and second ports, wherein the controller is configured to, when the connector of the assembly is attached to the first port:
obtain the stored information from the memory device;
make a determination based on the stored information that the assembly comprises the instrument indicator;
in response to said determination, control the first port indicator to display light of a first color and control the second port indicator to display light of a second color that is different from the first color;
control the instrument indicator to display light of the first color; and
control the medical instrument to perform a function for treatment of a patient.

2. The system of claim 1, wherein the controller is further configured to control the first and second port indicators to continue displaying light of the first and second colors, respectively, throughout said control of the medical instrument to perform the function for treatment of a patient.

3. The system of claim 1, wherein the controller is further configured to, when the connector of the assembly is subsequently detached from the first port, control the first and second port indicators to simultaneously display a third color that differs from each of the first and second colors.

4. The system of claim 1, further comprising an additional assembly that comprises:
an additional connector;
an additional medical instrument in electrical communication with the additional connector; and
an additional instrument indicator,
wherein the controller is further configured to, while the connector of the assembly remains attached to the first port and when the additional connector of the additional assembly is attached to the second port, control the additional instrument indicator to display light of the second color.

5. The system of claim 4, wherein the controller is further configured to, while the additional connector is attached to the second port, control the additional medical instrument to perform an additional function for treatment of a patient.

6. The system of claim 5, wherein the function performed by the medical instrument of the assembly and the additional function performed by the additional medical instrument of the additional assembly each comprises delivery of radiofrequency energy to the patient.

7. The system of claim 1, further comprising a further assembly that comprises a further connector and a further medical instrument in electrical communication with the further connector, the further assembly being devoid of any instrument indicator,
wherein the controller is further configured to, after the connector of the assembly has been detached from the first port and the further connector of the further assembly has been attached to either the first port or the second port:
make a determination that the further assembly is devoid of any instrument indicator; and
in response to said determination, control the first and second port indicators to display light of a third color that differs from each of the first and second colors.

8. The system of claim 7, wherein the third color is white.

9. The system of claim 7, wherein the further assembly further comprises a further memory device comprising further stored information, and wherein the controller is configured to obtain the further stored information from the further memory device and make a determination that the further assembly is devoid of any instrument indicator based on the further stored information thus obtained.

10. The system of claim 7, wherein the further assembly is devoid of any stored information indicative of an absence of an instrument indicator from the further assembly, and wherein the controller is further configured to:
attempt to obtain stored information indicative of an absence of an instrument indicator from the further assembly; and
make said determination that the further assembly is devoid of any instrument indicator based on an inability to obtain from the assembly any stored information indicative of said absence.

11. The system of claim 1, further comprising an actuator in communication with the controller, wherein the controller is configured to, upon actuation of the actuator, cause the first and second port indicators to cease displaying light.

12. The system of claim 11, wherein the controller is configured to, upon subsequent actuation of the actuator, cause the first and second port indicators to again display light of the first and second colors, respectively.

13. The system of claim 11, wherein the control machine comprises a touchscreen, and wherein the controller causes the touchscreen to display the actuator as a virtual button in response to said determination that the assembly comprises the instrument indicator.

14. The system of claim 13, wherein the control machine is configured to cause the touchscreen to display a plurality of different control screens on the touchscreen, and wherein the virtual button is present on at least multiple of said plurality of different control screens.

15. The system of claim 1, wherein the memory device comprises a read/write device.

16. The system of claim 1, wherein the memory device comprises a read only device.

17. The system of claim 1, wherein the memory device comprises an electrically erasable programmable read-only memory (EEPROM) device.

18. The system of claim 1, wherein the memory device comprises a radiofrequency identification (RFID) device.

19. The system of claim 1, further comprising an elongated cable extending between the connector and the instrument.

20. The system of claim 19, wherein the instrument indicator is physically secured to the assembly such that at least a portion of the instrument indicator is distal to a distal end of the elongated cable.
